(19) United States
(12) Patent Application Publication
(10) Pub. No.: US 2017/0209445 A1
(43) Pub. Date: Jul. 27, 2017

(54) KINASE INHIBITORS

(71) Applicants: Respivert Limited, Buckinghamshire (GB); Topivert Pharma Limited, London (GB)

(72) Inventor: Matthew Colin Thor Fyfe, London (GB)

(21) Appl. No.: 15/515,079

(22) PCT Filed: Oct. 1, 2015
(86) PCT No.: PCT/GB2015/052875

§ 371 Date: Mar. 28, 2017

(30) Foreign Application Priority Data
Oct. 1, 2014 (GB) 1417346.2
Jun. 18, 2015 (GB) 1510711.3

(51) Int. Cl. A61K 31/517; A61K 31/496; A61K 31/541; A61K 31/5355

(52) U.S. Cl. CPC A61K 31/517; A61K 31/5355; A61K 31/496; A61K 31/541

(57) ABSTRACT

Compounds of formula I,

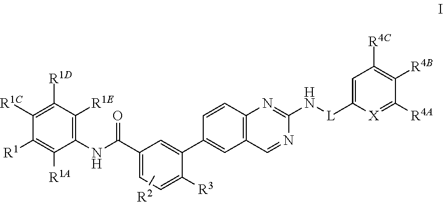

have anti-inflammatory activity. Exemplary mechanisms of action include inhibition of one or more of members of: the family of p38 mitogen-activated protein kinase enzymes; Syk kinase; and members of the Src family of tyrosine kinases. The compounds have use in therapy, including in pharmaceutical combinations, especially in the treatment of inflammatory diseases, including inflammatory diseases of the lung, eye and intestines.

This invention relates, inter alia, to compounds which are antiinflammatory agents (e.g. through inhibition of one or more of members of: the family of p38 mitogen-activated protein kinase enzymes (referred to herein as p38 MAP kinase inhibitors), for example the alpha kinase sub-type thereof; Syk kinase; and the Src family of tyrosine kinases). The invention also relates to the use of such compounds in therapy, including in mono- and combination therapies, especially in the treatment of inflammatory diseases, including inflammatory diseases of the lung (such as asthma and chronic obstructive pulmonary disease (COPD)), eye (such as uveitis or keratoconjunctivitis sicca (dry eye disease, also known as xerophthalmia)) and gastrointestinal tract (such as Crohn's disease and ulcerative colitis).

The listing or discussion of an apparently prior-published document in this specification should not necessarily be taken as an acknowledgement that the document is part of the state of the art or is common general knowledge.

Four p38 MAPK isoforms (alpha, beta, gamma and delta respectively) have been identified, each displaying different patterns of tissue expression. The p38 MAPK alpha and beta isoforms are found ubiquitously throughout the body; are present in many different cell types and are inhibited by a number of previously described small molecular weight compounds. Early classes of inhibitors were highly toxic due to the broad tissue distribution of these isoforms which resulted in off-target effects of the compounds. Some of the more recently identified inhibitors show improved selectivity for p38 MAPK alpha and beta isoforms and have wider safety margins.

p38 MAP kinase is believed to play a pivotal role in many of the signalling pathways that are involved in initiating and maintaining chronic, persistent inflammation in human disease, for example, in severe asthma, COPD and inflammatory bowel disease (IBD). There is now an abundant literature which demonstrates that p38 MAP kinase is activated by a range of pro-inflammatory cytokines and that its activation results in the recruitment and release of further pro-inflammatory cytokines. Indeed, data from some clinical studies demonstrate beneficial changes in disease activity in patients during treatment with p38 MAP kinase inhibitors. For instance Smith describes the inhibitory effect of p38 MAP kinase inhibitors on TNFα (but not IL-8) release from human PBMCs (Smith, S. J., Br. J. Pharmacol., 2006, 149:393-404).

The use of inhibitors of p38 MAP kinase in the treatment of COPD and IBD has also been proposed. Small molecule inhibitors targeted to p38 MAPKα/β have proved to be effective in reducing various parameters of inflammation in:
cells and tissues obtained from patients with COPD, who are generally corticosteroid insensitive (Smith, S. J., Br. J. Pharmacol., 2006, 149:393-404);
biopsies from IBD patients (Docena, G. et al., J. of Trans. Immunol., 2010, 162:108-115); and
in vivo animal models (Underwood, D. C. et al., Am. J. Physiol., 2000, 279:L895-902; Nath, P. et al., Eur. J. Pharmacol., 2006, 544:160-167).

Irusen and colleagues also suggested the possibility of involvement of p38 MAPKα/β on corticosteroid insensitivity via the reduction of binding affinity of the glucocorticoid receptor (GR) in nuclei (Irusen, E. et al., J. Allergy Clin. Immunol., 2002, 109:649-657). Clinical investigations in inflammatory diseases with a range of p38 MAP kinase inhibitors, including AMG548, BIRB 796, VX702, SCIO469 and SCIO323, has been described (Lee, M. R. and Dominguez, C., Current Med. Chem., 2005, 12:2979-2994.). However, the major obstacle hindering the utility of p38 MAP kinase inhibitors in the treatment of human chronic inflammatory diseases has been the toxicity observed in patients. This has been sufficiently severe to result in the withdrawal from clinical development of many of the compounds progressed, including all those specifically mentioned above.

COPD is a condition in which the underlying inflammation is reported to be substantially resistant to the anti-inflammatory effects of inhaled corticosteroids. Consequently, a superior strategy for treating COPD would be to develop an intervention which has both inherent anti-inflammatory effects and the ability to increase the sensitivity of the lung tissues of COPD patients to inhaled corticosteroids. The recent publication of Mercado et al. (2007; American Thoracic Society Abstract A56) demonstrates that silencing p38 MAPK γ has the potential to restore sensitivity to corticosteroids. Thus, there may be a dual benefit for patients in the use of a p38 MAP kinase inhibitor for the treatment of COPD.

Many patients diagnosed with asthma or with COPD continue to suffer from uncontrolled symptoms and from exacerbations of their medical condition that can result in hospitalisation. This occurs despite the use of the most advanced, currently available treatment regimens, comprising of combination products of an inhaled corticosteroid and a long acting β-agonist. Data accumulated over the last decade indicates that a failure to manage effectively the underlying inflammatory component of the disease in the lung is the most likely reason that exacerbations occur. Given the established efficacy of corticosteroids as anti-inflammatory agents and, in particular, of inhaled corticosteroids in the treatment of asthma, these findings have provoked intense investigation. Resulting studies have identified that some environmental insults invoke corticosteroid-insensitive inflammatory changes in patients' lungs. An example is the response arising from virally-mediated upper respiratory tract infections (URTI), which have particular significance in increasing morbidity associated with asthma and COPD.

It has been disclosed previously that compounds that inhibit the activity of both the c-Src and Syk kinases are effective agents against rhinovirus replication (Charron, C. E. et al., WO 2011/158042) and that compounds that inhibit p59-HCK are effective against influenza virus replication (Charron, C. E. et al., WO 2011/070369). Taken together with inhibition of p38 MAPK, these are particularly attractive properties for compounds to possess that are intended to treat patients with chronic respiratory diseases.

Certain p38 MAPK inhibitors have also been described as inhibitors of replication of respiratory syncytial virus (Cass L. et al., WO 2011/158039).

The precise etiology of IBD is uncertain, but is believed to be governed by genetic and environmental factors that interact to promote an excessive and poorly controlled mucosal inflammatory response directed against components of the luminal microflora. This response is mediated through infiltration of inflammatory neutrophils, dendritic cells and T-cells from the periphery. Due to the ubiquitous expression of p38 in inflammatory cells it has become an obvious target for investigation in IBD models. Studies investigating the efficacy of p38 inhibitors in animal models of IBD and human biopsies from IBD patients indicated that p38 could be a target for the treatment of IBD (Hove, T. ten et al., Gut, 2002, 50:507-512, Docena, G. et al., J. of Trans. Immunol., 2010, 162:108-115). However, these findings are not completely consistent with other groups reporting no effect with p38 inhibitors (Malamut G. et al., Dig. Dis. Sci, 2006, 51:1443-1453). A clinical study in Crohn's patients using the p38 alpha inhibitor BIRB796 demonstrated potential clinical benefit with an improvement in C-reactive protein levels. However this improvement was transient, returning to baseline by week 8 (Schreiber, S. et al., Clin. Gastro. Hepatology, 2006, 4:325-334). A small clinical study investigating the efficacy of CNI-1493, a P38 and Jnk inhibitor, in patients with severe Crohn's disease showed significant improvement in clinical score over 8 weeks (Hommes, D. et al. Gastroenterology. 2002 122:7-14).

T cells are known to play key role in mediating inflammation of the gastrointestinal tract. Pioneering work by Powrie and colleagues demonstrated that transfer of naive CD4+ cells into severly compromised immunodeficient (SCID) animals results in the development of colitis which is dependent on the presence of commensal bacteria (Powrie F. et al. Int Immunol. 1993 5:1461-71). Furthermore, investigation of mucosal membranes from IBD patients showed an upregulation of CD4+ cells which were either Th1 (IFNg/IL-2) or Th2 (IL5/TGFb) biased depending on whether the patient had Crohn's disease or ulcerative colitis (Fuss I J. et al. J Immunol. 1996 157:1261-70.). Similarly, T cells are known to play a key role in inflammatory disorders of the eye with several studies reporting increased levels of T cell associated cytokines (IL-17 and IL-23) in sera of Beçhets patients (Chi W. et al. Invest Ophthalmol Vis Sci. 2008 49:3058-64). In support, Direskeneli and colleagues demonstrated that Behcets patients have increased Th17 cells and decreased Treg cells in their peripheral blood (Direskeneli H. et al. J Allergy Clin Immunol. 2011 128:665-6).

One approach to inhibit T cell activation is to target kinases which are involved in activation of the T cell receptor signalling complex. Syk and Src family kinases are known to play a key role in this pathway, where Src family kinases, Fyn and Lck, are the first signalling molecules to be activated downstream of the T cell receptor (Barber E K. et al. PNAS 1989 86:3277-81). They initiate the tyrosine phosphorylation of the T cell receptor leading to the recruitment of the Syk family kinase, ZAP-70. Animal studies have shown that ZAP-70 knockout results in a SCID phenotype (Chan A C. et al. Science. 1994 10; 264(5165):1599-601).

A clinical trial in rheumatoid arthritis patients with the Syk inhibitor Fostamatinib demonstrated the potential of Syk as an anti-inflammatory target with patients showing improved clinical outcome and reduced serum levels of IL-6 and MMP-3 (Weinblatt M E. et al. Arthritis Rheum. 2008 58:3309-18). Syk kinase is widely expressed in cells of the hematopoietic system, most notably in B cells and mature T cells. Through interaction with immunoreceptor tyrosine-based activation (ITAM) motifs it plays an important role in regulating T cell and B cell expansion as well as mediating immune-receptor signalling in inflammatory cells. Syk activation leads to IL-6 and MMP release—inflammatory mediators commonly found upregulated in inflammatory disorders including IBD and rheumatoid arthritis (Wang Y D. et al. World J Gastroenterol 2007; 13: 5926-5932, Litinsky I et al. Cytokine. 2006 January 33:106-10).

In addition to playing key roles in cell signalling events which control the activity of pro-inflammatory pathways, kinase enzymes are now also recognised to regulate the activity of a range of cellular functions, including the maintenance of DNA integrity (Shilo, Y. Nature Reviews Cancer, 2003, 3: 155-168) and co-ordination of the complex processes of cell division. Indeed, certain kinase inhibitors (the so-called “Olaharsky kinases”) have been found to alter the frequency of micronucleus formation in vitro (Olaharsky, A. J. et al., PLoS Comput. Biol., 2009, 5(7)). Micronucleus formation is implicated in, or associated with, disruption of mitotic processes and is therefore undesirable. Inhibition of glycogen synthase kinase 3α (GSK3α) was found to be a particularly significant factor that increases the likelihood of a kinase inhibitor promoting micronucleus formation. Also, inhibition of the kinase GSK33 with RNAi has been reported to promote micronucleus formation (Tighe, A. et al., BMC Cell Biology, 2007, 8:34).

Whilst it may be possible to attenuate the adverse effects of inhibition of Olaharsky kinases such as GSK3α by optimisation of the dose and/or by changing the route of administration of a molecule, it would be advantageous to identify further therapeutically useful molecules with low or negligible inhibition of Olaharsky kinases, such as GSK 3α and/or have low or negligible disruption of mitotic processes (e.g. as measured in a mitosis assay).

Various kinase inhibitors are disclosed as having potential utility in the treatment of inflammatory conditions.

For example, compounds based upon quinazolines or phthalazines that are designed as orally bioavailable, selective inhibitors of Lck (a member of the Src family of kinases) or p38α, which compounds are described as having potential utility in the treatment of inflammatory diseases, are disclosed in: J. Med. Chem. 2006, 49, 5671-5686; ibid. 2008, 51, 1681-1694; and ibid. 2008, 51, 6271-6279. Further compounds based upon a core comprising a bicyclic, nitrogen-containing heteroaromatic ring, which compounds are described as inhibiting kinases and having utility, inter alia, in the treatment of inflammatory diseases, are disclosed in WO 2006/039718, WO 2006/118256, WO 2006/137421 and WO 2009/131173.

Other compounds that inhibit kinases include those based upon diaryl ureas (see, for example, WO 01/36403, WO 02/083628, WO 2014/027209, WO 2014/076484, WO 2014/140582, WO 2014/162121, WO 2014/162122, WO 2014/162126 and WO 2015/092423) or diaryl amides (see, for example, WO 2010/026095, WO 2010/026096 and J. Med. Chem. 50, 2007, 4016-4026).

Some compounds based upon diaryl amides are known to inhibit certain cytokines. Such compounds include those disclosed in, for example, WO 2003/087085, WO 2005/090333, WO 2007/056016, WO 2007/075896, WO 2008/021388, US 2004/0186114, US 2005/0245536 and US 2005/0256113.

Compounds that inhibit tyrosine kinases and that are presented as having utility in the treatment of conditions such as cancer, infections, inflammation and/or autoimmune diseases include those disclosed in WO 2010/026262 and WO 2010/094695.

Nevertheless, there remains a need to identify and develop new kinase inhibitors, specifically alternative p38 MAP kinase inhibitors that are suitable for the treatment of inflammation. There is particularly a need for such inhibitors that have improved therapeutic potential over currently available treatments or, in particular, that exhibit a superior therapeutic index (e.g. inhibitors that are at least equally efficacious and, in one or more respects, are less toxic at the relevant therapeutic dose than previous agents).

We have now discovered, surprisingly, that certain 2-arylamino-6-phenylquinazoline compounds inhibit one or more of p38 MAP kinase, Syk and Src family kinases and therefore possess good anti-inflammatory properties.

Thus, according to a first aspect of the invention, there is provided a compound of formula I,

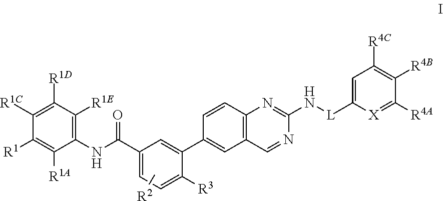

wherein
R¹ represents
-L¹-C(O)NR^{X}R^{Y},
-L²-S(O)₂R^{Y1},
-L³-P(O)R^{Y1}R^{Y2},
-L⁴-S(O)₂NR^{X}R^{Y},
-L⁵-C(O)R^{Y},
-L⁶-S(O)₀₋₁—R^{Y1},
-L⁷-C(O)OR^{Y},
—N═S(O)(CH₃)₂ or
—S(═O)(═NR^{X})CH₃
—NR^{X}R^{X1},
Het¹ optionally substituted with one or more substituents selected from halo, hydroxy, C₁₋₄ alkyl and C₁₋₄ alkoxy,
—[C₁₋₄ alkylene]₀₋₁-Het², which Het² group is optionally substituted with one or more substituents selected from halo, hydroxy, oxo, C₁₋₄ alkyl and C₁₋₄ alkoxy,
—[C₁₋₄ alkylene]₀₋₁—CN,
—C₁₋₄ alkylene-OH
—C₁₋₄ alkylene-OP(O)(OH)₂ or
halo
   or, when either one or none of R^{4A} to R^{4C} represents H, then R¹ may alternatively represent H;
   L¹, L², L³, L⁴, L⁵, L⁶ and L⁷ independently represent a bond, —[C(R^{a})(R^{b})]₁₋₂—, —C(R^{a})(R^{b})—N(R^{X2})—, where the C-atom is connected to the phenyl ring,
   or L¹, L², L³, L⁴, L⁵ or L⁷ represents —N(R^{X2})—,
   or L¹, L² or L⁴ represents —O—,
   or L³ or L⁷ represents —OC(R^{a})(R^{b})—, wherein the O-atom of the latter substituent is attached to the phenyl ring;
   R^{X} and R^{X1} independently represent H or C₁₋₆ alkyl optionally substituted by hydroxy or —OP(O)(OH)₂,
   or R^{X1} represents Het¹ optionally substituted with one or more substituents selected from halo, hydroxy, C₁₋₄ alkyl and C₁₋₄ alkoxy;
   R^{Y}, R^{Y1} and R^{Y2} independently represent C₁₋₆ alkyl, C₃₋₇ cycloalkyl, phenyl, benzyl, Het¹ or Het², which latter six groups are optionally substituted by one or more substituents selected from halo, hydroxy, —OP(O)(OH)₂, C₁₋₄ alkyl, C₁₋₄ alkoxy, C(O)OH, —N(R^{c})(R^{d}) and Het³, which latter group is optionally substituted by C₁₋₄ alkyl,
   or R^{Y} represents H,
   each R^{X2} independently represents H or C₁₋₄ alkyl optionally substituted by hydroxy or —OP(O)(OH)₂;
   R^{c} and R^{d} independently represent H, methyl or —C(R^{e})(R^{f})—C₁₋₃ alkyl, the C₁₋₃ alkyl portion of which latter group is optionally substituted by one or more hydroxy substituents;
   R^{a}, R^{b}, R^{e} and R^{f} represent, independently at each occurrence, H or methyl;
   R^{1A} represents
C₁₋₆ alkoxy, C₁₋₆ alkyl, C₂₋₆ alkenyl, C₂₋₆ alkynyl, which latter four groups are optionally substituted by one or more substituents selected from C₁₋₂ alkyl, halo, hydroxy, —OP(O)(OH)₂, C₁₋₂ alkoxy and —N(R^{c})(R^{d}) or Het⁴,
H, halo, cyano,
phenyl or Het¹, which latter two groups are optionally substituted with one or more substituents selected from C₁₋₄ alkyl and C₁₋₄ alkoxy;
   R^{1C} and R^{1E} independently represent H, halo, cyano or methyl;
   R^{1D} represents C₂₋₇ alkyl, C₂₋₇ alkenyl, C₂₋₇ alkynyl, C₃₋₇ cycloalkyl, phenyl, Het¹ or Het², which latter seven groups are optionally substituted by one or more substituents selected from C₁₋₄ alkyl, halo, cyano, hydroxy, —OP(O)(OH)₂ and C₁₋₄ alkoxy, or R^{1D} represents trimethylsilyl or trifluoromethyl;
   R² represents H, C₁₋₄ alkyl, C₃₋₄ cycloalkyl or halo;
   R³ represents C₁₋₂ alkyl optionally substituted by one or more halo atoms, or R³ represents H or halo;
   L represents a direct bond or C₁₋₂ alkylene;
   X represents CH or N;
   one of R^{4A}, R^{4B} and R^{4C} represents R^{5a}, and each of the other two of R^{4A}, R^{4B} and R^{4C} independently represents R^{5b};
   R^{5a} represents
-Q¹-C(R^{6c})(R^{6d})—[C₁₋₅ alkylene]-R^{6a}, which C₁₋₅ alkylene group is optionally substituted by oxo and/or by one or more substituents selected from halo and hydroxy,
-Q²-[C(R^{6c})(R^{6d})—(CH₂)₀₋₁CH₂—O]₁₋₁₂—CH₂(CH₂)₀₋₁CH₂—R^{6a},
-Q³-J-Het^{x}, which Het^{x} group is optionally substituted by one or more substituents selected from halo, hydroxyl, oxo, CO₂H, C₁₋₃ alkyl, C₁₋₃ alkoxy and C₁₋₃ hydroxyalkyl,
-J-S(O)ₙR^{6b},
-J-P(O) R^{6e}R^{6f},
-J-CO₂H,
—OS(O)₂R^{6e},
—OS(O)₂NH₂,
—N═S(O)R^{6e}R^{6f},
-J-COR^{6b},
—S(═O)(═NR^{6c})CH₃,
—OCONH₂,
—CH₂OH or
—CH₂OP(O)(OH)₂;
   R^{5b} represents C₁₋₃ alkoxy or C₁₋₃ alkyl, which latter two groups are optionally substituted by hydroxy, —OP(O)(OH)₂ or one or more halo atoms, or R^{5b} represents —S(O)₁₋₂R^{6e}, —OS(O)₂R^{6e}, —N(R⁹)(R¹⁰), C₂₋₃ alkynyl, H, cyano, —C(O)N(R⁹)(R¹⁰), hydroxy or halo;
   R^{6a} represents OR^{7a}, —S(O)₀₋₂R^{7aa}, —N(R^{7b})(R^{7c}) or CO₂H;
   R^{6b} represents C₁₋₈ alkyl, C₃₋₈ cycloalkyl, phenyl, Het¹ or Het², which latter five groups are optionally substituted by one or more substituents selected from halo, hydroxyl, —OP(O)(OH)₂, C₁₋₄ alkyl and C₁₋₄ alkoxy and which Het² group is optionally substituted or further substituted by one or more oxo groups,
   or, when n is 2 or R^{6b} is attached to -J-CO—, in which J is C₁₋₄ alkylene, R^{6b} may alternatively represent —N(R^{7b})(R^{7c});
   R^{7a} to R^{7C} independently represent H, —C(R^{7d})(R^{7e})—C₁₋₃ alkylene-OH, —C(R^{7d})(R^{7e})—C₁₋₃ alkylene-OP(O)(OH)₂ or C₁₋₄ alkyl optionally substituted by one or more halo atoms, or R^{7b} and R^{7c}, together with the N-atom to which they are attached, form a 4- to 7-membered heterocyclic group that is fully saturated, partially unsaturated or fully aromatic and which heterocyclic group contains one N atom (the atom to which R^{7b} and R^{7c} are attached) and, optionally, one or more further heteroatoms selected from O, S and N, and which heterocyclic group is optionally substituted by one or more substituents selected from halo, hydroxy, oxo, C₁₋₄ alkyl, C₁₋₄ alkoxy and C₁₋₄ hydroxyalkyl,
   or R^{7a} represents —P(O)(OH)₂;
   R^{7aa} represents —C(R^{7d})(R^{7e})—C₁₋₃ alkylene-OH or C₁₋₄ alkyl optionally substituted by one or more halo atoms;
   R^{6c}, R^{6d}, R^{7d} and R^{7e} independently represent H or methyl;
   R^{6e} and R^{6f} represent, independently at each occurrence, C₁₋₄ alkyl optionally substituted with one or more halo atoms,
   or, when R^{5a} represents -J-P(O)R^{6e}R^{6f}, R^{6e} and R^{6f} may additionally represent hydroxy or C₁₋₄ alkoxy;
   Q¹, Q², and Q³ independently represent —C(O)N(R⁸)—, —O—, —S(O)₂N(R⁸)— or —S(O)ₚ—;
   J represents a direct bond or C₁₋₄ alkylene;
   n and p independently represent 0, 1 or 2;
   R⁸, R⁹ and R¹⁰ independently represent H or methyl;
   Het^{x} represents Het¹ or Het³;
   Het¹ represents, independently upon each occurrence, a 5- or 6-membered heterocyclic group that is fully aromatic, which group contains one or more heteroatoms selected from N, O and S; and
   Het² to Het⁴ represent, independently upon each occurrence, a 4- to 7-membered heterocyclic group that is fully saturated or partially unsaturated, which group contains one or more heteroatoms selected from N, O and S;
   or a pharmaceutically acceptable salt, solvate or isotopic derivative thereof,
   which compounds may be referred to hereinafter as “the compounds of the invention”.

Pharmaceutically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals.

For the avoidance of doubt, compounds of formula I may contain the stated atoms in any of their natural or non-natural isotopic forms. In this respect, embodiments of the invention that may be mentioned include those in which:
(a) the compound of formula I is not isotopically enriched or labelled with respect to any atoms of the compound; and
(b) the compound of formula I is isotopically enriched or labelled with respect to one or more atoms of the compound.

References herein to an “isotopic derivative” relate to the second of these two embodiments. In particular embodiments of the invention, the compound of formula I is isotopically enriched or labelled (with respect to one or more atoms of the compound) with one or more stable isotopes. Thus, the compounds of the invention that may be mentioned include, for example, compounds of formula I that are isotopically enriched or labelled with one or more atoms such as deuterium or the like.

Compounds of formula I may exhibit tautomerism. All tautomeric forms and mixtures thereof are included within the scope of the invention. In particular, the invention includes the keto-enol tautomerism existing between indolin-2-one and 2-hydroxyindole.

Unless otherwise specified, alkyl groups and alkoxy groups as defined herein may be straight-chain or, when there is a sufficient number (i.e. a minimum of three) of carbon atoms, be branched. Particular alkyl groups that may be mentioned include, for example, methyl, ethyl, n-propyl, iso-propyl, butyl, n-butyl and tert-butyl. Particular alkoxy groups that may be mentioned include, for example, methoxy, ethoxy, propoxy, and butoxy.

Unless otherwise specified, cycloalkyl groups as defined herein may, when there is a sufficient number (i.e. a minimum of four) of carbon atoms, be part cyclic/acyclic.

Unless otherwise specified, alkylene groups as defined herein may be straight-chain or, when there is a sufficient number (i.e. a minimum of two) of carbon atoms, be branched. In particular embodiments of the invention, alkylene refers to straight-chain alkylene.

Unless otherwise stated, the point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like. Embodiments of the invention that may be mentioned include those in which aryl is phenyl.

For the avoidance of doubt, oxo substituents that may be present on heterocyclic groups represented by Het², Het³, Het⁴ and N(R^{7b})R^{7c} may be attached to any appropriate atoms in the heterocyclic ring including, where valencies allow, to C-, N- and/or S-atoms within the ring (thereby forming keto, N-oxide, S(O) and/or S(O)₂ groups).

Values of Het¹ that may be mentioned include imidazolyl (e.g. imidazol-1-yl or imidazol-5-yl) or pyridinyl (e.g. pyridin-3-yl).

Values of Het² that may be mentioned include thiomorpholinyl (e.g. thiomorpholin-4-yl).

Values of Het³ that may be mentioned include homomorpholinyl (e.g. homomorpholin-4-yl), morpholinyl (e.g. morpholin-4-yl), piperazinyl (e.g. piperazin-1-yl), piperidinyl (e.g. piperidin-1-yl or piperidin-4-yl), pyrrolidinyl (e.g. pyrrolidin-1-yl), tetrahydrothiopyranyl (e.g. tetrahydrothiopyran-4-yl) and thiomorpholinyl (e.g. thiomorpholin-4-yl).

Unless otherwise specified, the term “halo” includes references to fluoro, chloro, bromo or iodo, in particular to fluoro, chloro or bromo, especially fluoro or chloro.

Embodiments of the invention that may be mentioned include those in which:
(a) R¹ represents
   -L¹-C(O)NR^{X}R^{Y},
   -L²-S(O)₂R^{Y1},
   -L³-P(O)R^{Y1}R^{Y2},
   -L⁴-S(O)₂NR^{X}R^{Y},
   -L⁵-C(O)R^{Y},
   -L⁶-S(O)₀₋₁—R^{Y1},
   -L⁷-C(O)OR^{Y},
   —N═S(O)(CH₃)₂ or
   —S(═O)(═NR^{X})CH₃
   —NR^{X}R^{X},
   Het¹ optionally substituted with one or more substituents selected from halo, hydroxy, C₁₋₄ alkyl and C₁₋₄ alkoxy,
   —[C₁₋₄ alkylene]₀₋₁-Het², which Het² group is optionally substituted with one or more substituents selected from halo, hydroxy, oxo, C₁₋₄ alkyl and C₁₋₄ alkoxy,
   —[C₁₋₄ alkylene]₀₋₁—CN,
   —C₁₋₄ alkylene-OH or
   halo
   or, when either one or none of R^{4A} to R^{4C} represents H, then R¹ may alternatively represent H;
(b) L¹, L², L³, L⁴, L⁵, L⁶ and L⁷ independently represent a bond, —[C(R^{a})(R^{b})]₁₋₂—, —C(R^{a})(R^{b})—N(R^{X2})—, where the C-atom is connected to the phenyl ring,
   or L¹, L², L³, L⁴, L⁵ or L⁷ represents —N(R^{X2}),
   or L¹, L² or L⁴ represents —O—,
   or L³ represents —OC(R^{a})(R^{b})—, wherein the O-atom of the latter substituent is attached to the phenyl ring;
(c) R^{X} and R^{X1} independently represent H or C₁₋₆ alkyl,
   or R^{X1} represents Het¹ optionally substituted with one or more substituents selected from halo, hydroxy, C₁₋₄ alkyl and C₁₋₄ alkoxy;
   R^{Y}, R^{Y1} and R^{Y2} independently represent C₁₋₆ alkyl, C₃₋₇ cycloalkyl, phenyl, benzyl, Het¹ or Het², which latter six groups are optionally substituted by one or more substituents selected from halo, hydroxy, C₁₋₄ alkyl, C₁₋₄ alkoxy, C(O)OH, —N(R^{c})(R^{d}) and Het³, which latter group is optionally substituted by C₁₋₄ alkyl,
   or R^{Y} represents H,
   each R^{X2} independently represents H or C₁₋₄ alkyl;
(d) R^{1A} represents
   C₁₋₆ alkoxy, C₁₋₆ alkyl, C₂₋₆ alkenyl, C₂₋₆ alkynyl, which latter four groups are optionally substituted by one or more substituents selected from C₁₋₂ alkyl, halo, hydroxy, C₁₋₂ alkoxy and —N(R^{c})(R^{d}) or Het⁴,
   H, halo, cyano,
   phenyl or Het¹, which latter two groups are optionally substituted with one or more substituents selected from C₁₋₄ alkyl and C₁₋₄ alkoxy;
(e) R^{1D} represents C₂₋₇ alkyl, C₂₋₇ alkenyl, C₂₋₇ alkynyl, C₃₋₇ cycloalkyl, phenyl, Het¹ or Het², which latter seven groups are optionally substituted by one or more substituents selected from C₁₋₄ alkyl, halo, cyano, hydroxy and C₁₋₄ alkoxy, or R^{1D} represents trimethylsilyl or trifluoromethyl;
(f) R^{5a} represents
   -Q¹-C(R^{6c})(R^{6d})—[C₁₋₅ alkylene]-R^{6a}, which C₁₋₅ alkylene group is optionally substituted by oxo,
   -Q²-[C(R^{6c})(R^{6d})—(CH₂)₀₋₁CH₂—O]₁₋₁₂—CH₂(CH₂)₀₋₁CH₂—R^{6a},
   -Q³-J-Het³, which Het³ group is optionally substituted by one or more substituents selected from halo, hydroxyl, oxo, C₁₋₃ alkyl, C₁₋₃ alkoxy and C₁₋₃ hydroxyalkyl,
   -J-S(O)ₙR^{6b},
   -J-P(O) R^{6e}R^{6f},
   -J-CO₂H,
   —OS(O)₂R^{6e},
   —OS(O)₂NH₂,
   —N═S(O)R^{6e}R^{6f},
   -J-COR^{6b},
   —S(═O)(═NR^{6c})CH₃,
   —OCONH₂ or
   —CH₂OH;
(g) R^{5b} represents C₁₋₃ alkoxy or C₁₋₃ alkyl, which latter two groups are optionally substituted by one or more halo atoms, or R^{5b} represents —S(O)₁₋₂R^{6e}, —OS(O)₂R^{6e}, —N(R⁹)(R¹⁰), C₂₋₃ alkynyl, H, cyano, —C(O)N(R⁹)(R¹⁰), hydroxy or halo;
(h) R^{6a} represents OR^{7a}, —N(R^{7b})(R^{7c}) or CO₂H;
(i) R^{6b} represents C₁₋₈ alkyl, C₃₋₈ cycloalkyl, phenyl, Het¹ or Het², which latter five groups are optionally substituted by one or more substituents selected from halo, hydroxyl, C₁₋₄ alkyl and C₁₋₄ alkoxy;
(j) R^{7a} to R^{7c} independently represent H, —C(R^{7d})(R^{7e})—C₁₋₃ alkylene-OH or C₁₋₄ alkyl optionally substituted by one or more halo atoms, or R^{7b} and R^{7c}, together with the N-atom to which they are attached, form a 4- to 7-membered heterocyclic group that is fully saturated, partially unsaturated or fully aromatic and which heterocyclic group contains one N atom (the atom to which R^{7b} and R^{7c} are attached) and, optionally, one or more further heteroatoms selected from O, S and N, and which heterocyclic group is optionally substituted by one or more substituents selected from halo, hydroxy, oxo, C₁₋₄ alkyl, C₁₋₄ alkoxy and C₁₋₄ hydroxyalkyl; and
(k) R^{6e} and R^{6f} represent, independently at each occurrence, C₁₋₄ alkyl optionally substituted with one or more halo atoms.

Embodiments of the invention that may be mentioned include those in which one or more of the following definitions apply to the compounds of formula I:
(a) R¹ represents —C₁₋₄ alkylene-OP(O)(OH)₂;
(b) L⁷ represents —OC(R^{a})(R^{b})—, wherein the O-atom of the latter substituent is attached to the phenyl ring;
(c) R^{X} and/or R^{X1} represents C₁₋₆ alkyl substituted by hydroxy or —OP(O)(OH)₂;
(d) R^{Y}, R^{Y1} and/or R^{Y2} represents C₁₋₆ alkyl, C₃₋₇ cycloalkyl, phenyl, benzyl, Het¹ or Het², which latter six groups are substituted by —OP(O)(OH)₂ and are optionally further substituted by one or more substituents selected from halo, hydroxy, —OP(O)(OH)₂, C₁₋₄ alkyl, C₁₋₄ alkoxy, C(O)OH, —N(R^{c})(R^{d}) and Het³, which latter group is optionally substituted by C₁₋₄ alkyl;
(e) R^{X2} represents C₁₋₄ alkyl substituted by hydroxy or —OP(O)(OH)₂;
(f) R^{1A} represents C₁₋₆ alkoxy, C₁₋₆ alkyl, C₂₋₆ alkenyl, C₂₋₆ alkynyl, which latter four groups are substituted by —OP(O)(OH)₂ and are optionally further substituted by one or more substituents selected from C₁₋₂ alkyl, halo, hydroxy, —OP(O)(OH)₂, C₁₋₂ alkoxy and —N(R^{c})(R^{d}) or Het⁴;
(g) R^{1D} represents C₂₋₇ alkyl, C₂₋₇ alkenyl, C₂₋₇ alkynyl, C₃₋₇ cycloalkyl, phenyl, Het¹ or Het², which latter seven groups are substituted by —OP(O)(OH)₂ and are optionally further substituted by one or more substituents selected from C₁₋₄ alkyl, halo, cyano, hydroxy, —OP(O)(OH)₂ and C₁₋₄ alkoxy;
(h) R^{5a} represents
   -Q¹-C(R^{6c})(R^{6d})—[C₁₋₅ alkylene]-R^{6a}, which C₁₋₅ alkylene group is substituted by one or more substituents selected from halo and hydroxy and is optionally further substituted by oxo,
   -Q³-J-Het¹, which Het¹ group is optionally substituted by one or more substituents selected from halo, hydroxyl, oxo, CO₂H, C₁₋₃ alkyl, C₁₋₃ alkoxy and C₁₋₃ hydroxyalkyl,
   -Q³-J-Het³, which Het³ group is substituted by CO₂H and is optionally further substituted by one or more substituents selected from halo, hydroxyl, oxo, CO₂H, C₁₋₃ alkyl, C₁₋₃ alkoxy and C₁₋₃ hydroxyalkyl or
   —CH₂OP(O)(OH)₂;
(i) R^{5b} represents C₁₋₃ alkoxy or C₁₋₃ alkyl, which latter two groups are substituted by hydroxy or —OP(O)(OH)₂;
(j) R^{6a}—S(O)₀₋₂R^{7aa};
(k) R^{6b} represents
   C₁₋₈ alkyl, C₃₋₈ cycloalkyl, phenyl, Het¹ or Het², which latter five groups are substituted by —OP(O)(OH)₂ and are optionally further substituted by one or more substituents selected from halo, hydroxyl, —OP(O)(OH)₂, C₁₋₄ alkyl and C₁₋₄ alkoxy and which Het² group is optionally substituted or further substituted by one or more oxo groups,
   Het² substituted by one or more oxo groups and optionally further substituted by one or more substituents selected from halo, hydroxyl, —OP(O)(OH)₂, C₁₋₄ alkyl and C₁₋₄ alkoxy
   or, when n is 2 or R^{6b} is attached to -J-CO—, in which J is C₁₋₄ alkylene, R^{6b} may alternatively represent —N(R^{7b})(R^{7c});
(l) R^{7a}, R^{7b} and/or R^{7c} represents —C(R^{7d})(R^{7e})—C₁₋₃ alkylene-OP(O)(OH)₂,
   or R^{7a} represents —P(O)(OH)₂; and/or
(m) when R^{5a} represents -J-P(O)R^{6e}R^{6f}, R^{6e} and/or R^{6f} represents hydroxy or C₁₋₄ alkoxy.

Embodiments of the invention that may be mentioned include those in which at most one of R^{4A}, R^{4B} and R^{4C} is H. In such embodiments, R^{5b}, when not H, may be, for example:
C₁₋₃ alkoxy or C₁₋₃ alkyl (e.g. C₂₋₃ alkyl), which latter two groups are optionally substituted by one or more halo atoms,
—N(R⁹)(R¹⁰), C₂₋₃ alkynyl, cyano, —C(O)N(R⁹)(R¹⁰), hydroxy or halo
(e.g. R^{5b} may be C₂₋₃ alkynyl, cyano, —C(O)N(R⁹)(R¹⁰) or, particularly, C₁₋₂ alkoxy optionally substituted by one or more halo atoms).

Embodiments of the invention that may be mentioned include compounds of formula I in relation to which one or more of the following apply:
(a) R¹ represents
   -L¹-C(O)NR^{X}R^{Y},
   -L²-S(O)₂R^{Y1},
   -L³-P(O) R^{Y1}R^{Y2},
   -L⁴-S(O)₂NR^{X}R^{Y},
   -L⁵-C(O) R^{Y},
   -L⁶-S(O)—R^{Y1},
   -L⁷-C(O)OR^{Y},
   —N═S(O)(CH₃)₂ or
   —S(═O)(═NR^{X})CH₃
   —NR^{X}R^{X1},
   Het¹ optionally substituted with one or more substituents selected from halo, hydroxy, C₁₋₂ alkyl and C₁₋₂ alkoxy,
   —[C₁₋₃ alkylene]₀₋₁-Het², which Het² group is optionally substituted with one or more substituents selected from halo, hydroxy, oxo, C₁₋₂ alkyl and C₁₋₂ alkoxy,
   —[C₁₋₃ alkylene]₀₋₁—CN,
   —C₁₋₄ alkylene-OH or
or, when either one or none of R^{4A} to R^{4C} represents H, then R¹ may alternatively represent H;
(b) L¹ and L⁶ independently represent —CH₂— or, particularly, a bond;
(c) L² and L⁷ independently represent a bond, —CH₂— or, particularly, —NH—;
(d) L³ represents —OCH₂—, —CH₂— or, particularly, a bond;
(e) L⁴ represents a bond or —NH—;
(f) L⁵ represents —CH₂— or, particularly, —CH₂—N(R^{X2})—, where the C-atom is connected to the phenyl ring;
(g) R^{X} represents H or methyl,
(h) R^{Y} represents C₁₋₄ alkyl or Het², which latter two groups are optionally substituted by one or more substituents selected from halo, hydroxy, C₁₋₃ alkyl, C₁₋₃ alkoxy, C(O)OH, —N(R^{c})(R^{d}) and Het³, which latter group is optionally substituted by C₁₋₃ alkyl, or R^{Y} represents H;
(i) R^{X1} represents H, methyl or Het¹, which latter group is optionally substituted with one or more substituents selected from halo, hydroxy, C₁₋₂ alkyl and C₁₋₂ alkoxy;
(j) R^{X2} represents H or methyl;
(k) R^{Y1} and R^{Y2} independently represent C₁₋₃ alkyl;
(l) R^{1A} represents halo, cyano, C₁₋₃ alkoxy or C₁₋₃ alkyl, which latter two groups are optionally substituted by one or more substituents selected from methoxy and —N(R^{c})(R^{d}) or R^{1A} represents Het¹ optionally substituted with one or more substituents selected from C₁₋₂ alkyl and C₁₋₂ alkoxy;
(m) R^{c} and R^{d} independently represent H or methyl;
(n) R^{1C} and R^{1E} both represent H;
(o) R^{1D} represents trifluoromethyl or, particularly, trimethylsilyl, C₂₋₅ alkyl, C₂₋₅ alkynyl, C₃₋₆ cycloalkyl, Het¹ or Het², which latter five groups are optionally substituted by one or more substituents selected from halo, cyano, hydroxy and C₁₋₂ alkoxy;
(p) R² represents halo or, particularly, H;
(q) R³ represents C₁₋₂ alkyl optionally substituted by one or more halo atoms;
(r) L represents CH₂ or, particularly, a direct bond;
(s) X represents N or CH;
(t) either
   R^{4A} represents R^{5a} and R^{4B} and R^{4C} independently represent R^{5b} or
   R^{4B} represents R^{5a} and R^{4A} and R^{4C} independently represent R^{5b};
(u) R^{5a} represents
   -Q¹-C(R^{6c})(R^{6d})—[C₁₋₄ alkylene]-R^{6a}, which C₁₋₃ alkylene group is optionally substituted by oxo or one or more hydroxy,
   -Q²-[C(R^{6c})(R^{6d})—(CH₂)₀₋₁CH₂—O]₁₋₆—CH₂(CH₂)₀₋₁CH₂—R^{6a},
   -Q³-J-Het^{X}, which Het^{x} group is optionally substituted by one or more substituents selected from halo, hydroxyl, oxo, C₁₋₃ alkyl, C₁₋₃ alkoxy and C₁₋₃ hydroxyalkyl,
   -J-S(O)ₙR^{6b},
   -J-P(O)R^{6e}R^{6f},
   -J-CO₂H,
   —OS(O)₂CH₃,
   —N═S(O)(CH₃)₂,
   -J-COR^{6b} or
   —S(═O)(═NH)CH₃
(e.g. R^{5a} represents
   -Q¹-C(R^{6c})(R^{6d})—[C₁₋₃ alkylene]-R^{6a}, which C₁₋₃ alkylene group is optionally substituted by oxo,
   -Q²-[C(R^{6c})(R^{6d})—(CH₂)₀₋₁CH₂—O]₁₋₆—CH₂(CH₂)₀₋₁CH₂—R^{6a},
   -Q³-J-Het³, which Het³ group is optionally substituted by one or more substituents selected from halo, hydroxyl, oxo, C₁₋₃ alkyl, C₁₋₃ alkoxy and C₁₋₃ hydroxyalkyl,
   -J-S(O)ₙR^{6b},
   -J-P(O)R^{6e}R^{6f} or
   -J-CO₂H);
(v) R^{5b} represents C₁₋₃ alkoxy or C₁₋₃ alkyl, which latter two groups are optionally substituted by one or more halo atoms, or R^{5b} represents —OS(O)₂CH₃, —N(R⁹)(R¹⁰), C₂₋₃ alkynyl, H, cyano, —C(O)N(R⁹)(R¹⁰), hydroxy or halo
(e.g. R^{5b} represents C₁₋₃ alkoxy or C₁₋₃ alkyl, which latter two groups are optionally substituted by one or more halo atoms, or R^{5b} represents —N(R⁹)(R¹⁰), C₂₋₃ alkynyl, H, cyano, —C(O)N(R⁹)(R¹⁰), hydroxy or halo);
(w) R^{6a} represents —S(O)₂—C₁₋₂ alkyl, which alkyl group is optionally substituted by one or more halo atoms or, particularly, R^{6a} represents OR^{7a}, —N(R^{7b})(R^{7c}) or CO₂H;
(x) R^{6b} represents
   C₁₋₆ alkyl, C₃₋₆ cycloalkyl, phenyl, Het¹ or Het², which latter five groups are optionally substituted by one or more substituents selected from halo, hydroxyl, C₁₋₂ alkyl and C₁₋₂ alkoxy and which Het² group is optionally substituted or further substituted with one or more oxo groups;
(y) R^{7a} to R^{7c} independently represent H, —C(R^{7d})(R^{7e})—C₁₋₂ alkylene-OH or C₁₋₂ alkyl optionally substituted by one or more halo atoms, or R^{7b} and R^{7c}, together with the N-atom to which they are attached, form a 4- to 6-membered heterocyclic group that is fully saturated, partially unsaturated or fully aromatic and which heterocyclic group contains one N atom (the atom to which R^{7b} and R^{7c} are attached) and, optionally, one or more further heteroatoms selected from O, S and N, and which heterocyclic group is optionally substituted by one or more substituents selected from halo, hydroxy, oxo, C₁₋₂ alkyl, C₁₋₂ alkoxy and C₁₋₂ hydroxyalkyl;
(z) R^{6c}, R^{6d}, R^{7d} and R^{7e} independently represent H or methyl;
(aa) R^{6e} and R^{6f} independently represent C₁₋₃ alkyl;
(ab) Q¹, Q², and Q³ independently represent —C(O)N(R⁸)—, —O—, —S(O)₂N(R⁸)— or —S(O)₁₋₂—;
(ac) J represents a direct bond or C₁₋₃ alkylene;
(ad) m and n independently represent 0, 1 or 2;
(ae) p represents 1 or 2;
(af) R⁸, R⁹ and R¹⁰ independently represent H or methyl.

Embodiments of the invention that may be mentioned include those in which the compound of formula I is a compound of formula Ia, Ib, Ic, Id or Ie:

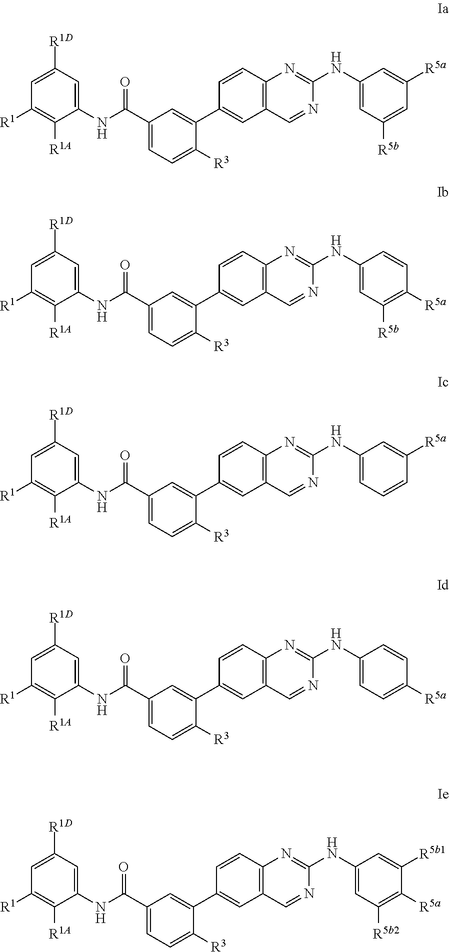

wherein R^{5b1} and R^{5b2} independently represent R^{5b} and R¹, R^{1A}, R^{1D}, R³, R^{5a} and R^{5b} are as hereinbefore defined.

Further embodiments of the invention that may be mentioned include those in which, in the compound of formula Ia, Ib, Ic, Id or Ie, R¹ represents:
-L¹-C(O)NR^{X}R^{Y} (e.g. —C(O)NR^{X}R^{Y}),
-L²-S(O)₂R^{Y1} (e.g. —N(R^{X2})—S(O)₂R^{Y1} or —S(O)₂R^{Y1}),
-L³-P(O)R^{Y1}R^{Y2} (e.g. —P(O)R^{Y1}R^{Y2}) or
-L⁶-S(O)—R^{Y1} (e.g. —S(O)—R^{Y1}).

Embodiments of the invention that may be mentioned include those in which one or more of the following definitions apply to the compounds of formula Ia, Ib, Ic, Id or Ie:
(a) R¹ represents —S(O)₁₋₂CH₃ or, particularly, —C(O)N(H)R^{Y}, —NHS(O)₂CH₃ or —P(O)(CH₃)₂;
(b) R^{Y} represents H;
(c) R^{1A} represents ethoxy or, particularly, methoxy;
(d) R^{1D} represents trifluoromethyl or, particularly, tert-butyl;
(e) R³ represents methyl;
(f) R^{5a} represents
   —C(O)NH—C(H)(R^{6c})—[C₁₋₄ alkylene]-R^{6a}, which C₁₋₃ alkylene group is optionally substituted by oxo or by one or two hydroxy (e.g. —C(O)NH—C(H)(CH₃)CH₂—R^{6a}, —C(O)NH—CH₂C(CH₃)₂—R^{6a}, —C(O)NH—CH₂CH₂CH₂—R^{6a}, —C(O)NH—CH₂C(O)—R^{6a}, —C(O)NH—CH₂C(CH₂OH)₂CH₂—R^{6a} or, particularly, —C(O)NH—CH₂CH₂—R^{6a}),
   -Q²-[C(H)(R^{6c})CH₂—O]₁₋₆—CH₂CH₂—R^{6a},
   -Q³-J-Het^{x},
   -J-P(O)(C₁₋₂ alkyl)₂,
   —CO₂H,
   —OS(O)₂CH₃,
   —N═S(O)(CH₃)₂,
   —CO-Het², which Het² group is optionally substituted by one or two oxo groups, or
   —S(═O)(═NH)CH₃
(e.g. R^{5a} represents
   —C(O)NH—C(H)(R^{6c})—[C₁₋₃ alkylene]-R^{6a}, which C₁₋₃ alkylene group is optionally substituted by oxo (e.g. —C(O)NH—C(H)(CH₃)CH₂—R^{6a}, —C(O)NH—CH₂C(CH₃)₂—R^{6a}, —C(O)NH—CH₂CH₂CH₂—R^{6a}, —C(O)NH—CH₂C(O)—R^{6a} or, particularly, —C(O)NH—CH₂CH₂—R^{6a}) or
   -Q²-[C(H)(R^{6c})CH₂—O]₁₋₆—CH₂CH₂—R^{6a});
(g) R^{5b} represents halo (e.g. fluoro), cyano, —C≡CH, —OS(O)₂CH₃, methyl or methoxy, which latter two groups are optionally substituted by one or more fluoro atoms
(e.g. R^{5b} represents —C≡CH or, particularly, methoxy, which latter group may be substituted by one or more fluoro atoms (to give, for example, OCH₃ or OCHF₂) or, particularly, may be unsubstituted);
(h) Q² represents —O— or —C(O)NH—;
(i) Q³ represents —O—, —C(O)N(H)—, —C(O)N(CH₃)— or —S(O)₂N(CH₃)—;
(j) R⁶ represents methyl or, particularly, H;
(k) R^{6a} represents —OH, —OCH₃—S(O)₂CH₃, —CO₂H or —N(R^{7b})(R^{7c}) (e.g. R^{6a} represents —OH, —CO₂H or, particularly, —OCH₃ or —N(R^{7b})(R⁷));
(l) R^{7b} and R^{7c} both represent methyl, or R^{7b} and R^{7c}, together with the N-atom to which they are attached, form a morpholinyl, thiomorpholinyl, homomorpholinyl or pyrrolidinyl group, or an optionally substituted piperazinyl or piperidinyl group (e.g. a piperazinyl group optionally substituted by one or more substituents selected from methyl and 2-hydroxyethyl, a piperidinyl group optionally substituted by hydroxy or a thiomorpholinyl group optionally substituted by one or more oxo groups)
(for example, R^{7b} and R^{7c}, together with the N-atom to which they are attached, form a piperazinyl group optionally substituted by methyl, a pyrrolidinyl group or a morpholinyl group (e.g. a piperazinyl group optionally substituted by methyl or, particularly, a morpholinyl group));
(m) J represents a direct bond or C₁₋₃ alkylene (e.g. a direct bond, CH₂ or C₂₋₃ n-alkylene);
(n) Het² represents a 5- or 6-membered heterocyclic group that is fully saturated or partially unsaturated, which group contains one or two heteroatoms selected from N, O and S.

Particular compounds of formula Ia, Ib, Ic, Id or Ie that may be mentioned include those in which:
(a) R¹ represents —C(O)NH₂, —P(O)(CH₃)₂ or, particularly, —NHS(O)₂CH₃;
(b) R^{1A} represents methoxy;
(d) R^{1D} represents tert-butyl;
(e) R² represents H; and
(f) R³ represents methyl.

Other compounds of formula Ia, Ib, Ic, Id or Ie that may be mentioned include the compounds of the examples described hereinafter. Thus, embodiments of the invention that may be mentioned include those in which the compound of formula Ia, Ib, Ic, Id or Ie is a compound selected from the list comprising:
3-(2-((3-methoxy-5-(2-(2-(2-methoxyethoxy)ethoxy)ethoxy)phenyl)amino)quinazolin-6-yl)-4-methyl-N-(3-(trifluoromethyl)phenyl)benzamide;
N-(5-(tert-butyl)-2-methoxy-3-(methylsulfonamido)phenyl)-3-(2-((3-methoxy-5-(2-(2-(2-methoxyethoxy)ethoxy)ethoxy)phenyl)amino)quinazolin-6-yl)-4-methylbenzamide;
N-(5-(tert-butyl)-3-carbamoyl-2-methoxyphenyl)-3-(2-((3-methoxy-5-((2-morpholinoethyl)carbamoyl)phenyl)amino)quinazolin-6-yl)-4-methylbenzamide;
N-(5-(tert-butyl)-3-(dimethylphosphoryl)-2-methoxyphenyl)-3-(2-((3-methoxy-5-((2-morpholinoethyl)carbamoyl)phenyl)amino)quinazolin-6-yl)-4-methylbenzamide;
N-(5-(tert-butyl)-2-methoxy-3-(methylsulfonamido)phenyl)-3-(2-((3-methoxy-5-((2-morpholinoethyl)carbamoyl)phenyl)amino)quinazolin-6-yl)-4-methylbenzamide;
N-(5-tert-butyl-2-methoxy-3-methylsulfonyl-phenyl)-3-[2-[3-methoxy-5-(2-morpholinoethyl-carbamoyl)anilino]quinazolin-6-yl]-4-methyl-benzamide;
N-(5-tert-butyl-2-methoxy-3-methylsulfinyl-phenyl)-3-[2-[3-methoxy-5-(2-morpholinoethyl-carbamoyl)anilino]quinazolin-6-yl]-4-methyl-benzamide;
N-(5-(tert-butyl)-2-methoxy-3-(methylsulfonamido)phenyl)-3-(2-((4-((dimethyl(oxo)-6-sulfanylidene)amino)phenyl)amino)quinazolin-6-yl)-4-methylbenzamide;
4-((6-(5-((5-(tert-butyl)-2-methoxy-3-(methylsulfonamido)phenyl)carbamoyl)-2-methylphenyl)quinazolin-2-yl)amino)-2-methoxybenzoic acid;
4-((6-(5-((5-(tert-butyl)-2-methoxy-3-(methylsulfonamido)phenyl)carbamoyl)-2-methylphenyl) quinazolin-2-yl)amino)-2-methoxy-N-methyl-N-(2-morpholinoethyl)benzamide;
3-((6-(5-((5-(tert-butyl)-2-methoxy-3-(methylsulfonamido)phenyl)carbamoyl)-2-methyl phenyl)-quinazolin-2-yl)amino)-5-((2-morpholinoethyl)carbamoyl)phenyl methanesulfonate;
4-((6-(5-((5-(tert-butyl)-2-methoxy-3-(methylsulfonamido)phenyl)carbamoyl)-2-methylphenyl)-quinazolin-2-yl)amino)-2-methoxy-N-(3-(1-oxidothiomorpholino)propyl)benzamide;
N-(5-(tert-butyl)-2-methoxy-3-(methylsulfonamido)phenyl)-3-(2-((4-(1,1-dioxidothiomorpholine-4-carbonyl)-3-methoxyphenyl)amino)quinazolin-6-yl)-4-methylbenzamide;
4-((6-(5-((5-(tert-butyl)-2-methoxy-3-(methylsulfonamido)phenyl)carbamoyl)-2-methylphenyl)-quinazolin-2-yl)amino)-N-(2-(4-(2-hydroxyethyl) piperazin-1-yl)ethyl)-2-methoxybenzamide;
3-((6-(5-((5-(tert-butyl)-2-methoxy-3-(methylsulfonamido)phenyl)carbamoyl)-2-methylphenyl)quinazolin-2-yl)amino)phenyl methanesulfonate;
N-(5-(tert-butyl)-2-methoxy-3-(methylsulfonamido)phenyl)-3-(2-((3-cyano-5-(2-morpholinoethoxy)phenyl)amino)quinazolin-6-yl)-4-methylbenzamide;
N-(5-(tert-butyl)-2-methoxy-3-(methylsulfonamido)phenyl)-3-(2-((3-methoxy-5-(N-methyl-N-(2-morpholinoethyl)sulfamoyl)phenyl)amino)quinazolin-6-yl)-4-methylbenzamide;
N-(5-(tert-butyl)-2-methoxy-3-(methylsulfonamido)phenyl)-4-methyl-3-(2-((3-((2-morpholinoethyl)carbamoyl)phenyl)amino)quinazolin-6-yl)benzamide;
N-(5-(tert-butyl)-2-methoxy-3-(methylsulfonamido)phenyl)-3-(2-((3-methoxy-5-(N-methyl-N-(3-morpholinopropyl)sulfamoyl)phenyl)amino)quinazolin-6-yl)-4-methylbenzamide;
N-(5-(tert-butyl)-2-methoxy-3-(methylsulfonamido)phenyl)-3-(2-((3-cyano-5-(2-(2-(2-methoxyethoxy)ethoxy)ethoxy)phenyl)amino)quinazolin-6-yl)-4-methylbenzamide;
N-(5-(tert-butyl)-2-methoxy-3-(methylsulfonamido)phenyl)-3-(2-((3-ethynyl-5-((2-(2-(2-methoxyethoxy)ethoxy)ethyl)carbamoyl)phenyl)amino)quinazolin-6-yl)-4-methylbenzamide;
N-(5-(tert-butyl)-2-methoxy-3-(methylsulfonamido)phenyl)-3-(2-((3-(dimethylphosphoryl)phenyl)amino)quinazolin-6-yl)-4-methylbenzamide;
N-(5-(tert-butyl)-2-methoxy-3-(methylsulfonamido)phenyl)-3-(2-((3-((dimethylphosphoryl)methyl)phenyl)amino)quinazolin-6-yl)-4-methylbenzamide;
4-((6-(5-((5-(tert-butyl)-2-methoxy-3-(methylsulfonamido)phenyl)carbamoyl)-2-methylphenyl)quinazolin-2-yl)amino)phenyl methanesulfonate;
N-(5-(tert-butyl)-2-methoxy-3-(methylsulfonamido)phenyl)-3-(2-((3-ethynyl-5-(2-(2-(2-methoxyethoxy)ethoxy)ethoxy)phenyl)amino)quinazolin-6-yl)-4-methylbenzamide;
N-(5-(tert-butyl)-2-methoxy-3-(methylsulfonamido)phenyl)-3-(2-((3-fluoro-5-(2-(2-(2-methoxyethoxy)ethoxy)ethoxy)phenyl)amino)quinazolin-6-yl)-4-methylbenzamide;
N-(5-(tert-butyl)-2-methoxy-3-(methylsulfonamido)phenyl)-3-(2-((3-ethynyl-5-((2-(2-methoxyethoxy)ethyl)carbamoyl)phenyl)amino)quinazolin-6-yl)-4-methylbenzamide;
N-(5-(tert-butyl)-2-methoxy-3-(methylsulfonamido)phenyl)-3-(2-((3-ethynyl-5-((2-methoxyethyl)carbamoyl)phenyl)amino)quinazolin-6-yl)-4-methylbenzamide;
3-((6-(5-((5-(tert-butyl)-2-methoxy-3-(methylsulfonamido)phenyl)carbamoyl)-2-methylphenyl)quinazolin-2-yl)amino)-5-methoxybenzoic acid;
4-((6-(5-((5-(tert-butyl)-2-methoxy-3-(methylsulfonamido)phenyl)carbamoyl)-2-methylphenyl)-quinazolin-2-yl)amino)-2-methoxy-N-(2-(2-(2-methoxyethoxy)ethoxy)ethyl)benzamide;
N-(5-(tert-butyl)-2-methoxy-3-(methylsulfonamido)phenyl)-3-(2-((3-((dimethyl(oxo)-I6-sulfanylidene)amino)phenyl)amino)quinazolin-6-yl)-4-methyl benzamide;
N-(5-(tert-butyl)-2-methoxy-3-(methylsulfonamido)phenyl)-3-(2-((3-(2-(2-(2-methoxyethoxy)-ethoxy)ethoxy)-5-(trifluoromethoxy)phenyl)amino)quinazolin-6-yl)-4-methylbenzamide;
N-(5-(tert-butyl)-2-methoxy-3-(methylsulfonamido)phenyl)-3-(2-((3-(2-(2-(2-methoxyethoxy)ethoxy)ethoxy)-5-(trifluoromethyl)phenyl)amino)quinazolin-6-yl)-4-methylbenzamide;
N-(5-(tert-butyl)-2-methoxy-3-(methylsulfonamido)phenyl)-4-methyl-3-(2-((4-(S-methylsulfonimidoyl)phenyl)amino)quinazolin-6-yl)benzamide;
N-(5-(tert-butyl)-2-methoxy-3-(methylsulfonamido)phenyl)-4-methyl-3-(2-((3-((2-morpholinoethyl)carbamoyl)-5-(trifluoromethyl)phenyl)amino)quinazolin-6-yl)benzamide;
4-((6-(5-((5-(tert-butyl)-2-methoxy-3-(methylsulfonamido)phenyl)carbamoyl)-2-methylphenyl) quinazolin-2-yl)amino)-N-(2-(2-(2-hydroxyethoxy)ethoxy)ethyl)-2-methoxybenzamide;
4-((6-(5-((5-(tert-butyl)-2-methoxy-3-(methylsulfonamido)phenyl)carbamoyl)-2-methylphenyl) quinazolin-2-yl)amino)-2-methoxy-N-(2-(2-methoxyethoxy)ethyl)benzamide;
4-((6-(5-((5-(tert-butyl)-2-methoxy-3-(methylsulfonamido)phenyl)carbamoyl)-2-methyl phenyl)-quinazolin-2-yl)amino)-N-(3-hydroxy-2,2-bis(hydroxymethyl)propyl)-2-methoxybenzamide;
N-(2-(1,4-oxazepan-4-yl)ethyl)-4-((6-(5-((5-(tert-butyl)-2-methoxy-3-(methylsulfonamido)-phenyl)carbamoyl)-2-methylphenyl)quinazolin-2-yl)amino)-2-methoxybenzamide;
4-((6-(5-((5-(tert-butyl)-2-methoxy-3-(m ethylsulfonamido)phenyl)carbamoyl)-2-methyl-phenyl)quinazolin-2-yl)amino)-2-methoxy-N-(2-(4-methylpiperazin-1-yl)ethyl)benzamide;
4-((6-(5-((5-(tert-butyl)-2-methoxy-3-(m ethylsulfonamido)phenyl)carbamoyl)-2-methyl-phenyl)quinazolin-2-yl)amino)-2-methoxy-N-(2-(1-methylpiperidin-4-yl)ethyl)benzamide;
N-(3-(1,4-oxazepan-4-yl)propyl)-4-((6-(5-((5-(tert-butyl)-2-methoxy-3-(methylsulfonamido)-phenyl)carbamoyl)-2-methylphenyl)quinazolin-2-yl)amino)-2-methoxybenzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(1-methylpiperidin-4-yl)benzamide;
4-((6-(5-((5-(tert-butyl)-2-methoxy-3-(methylsulfonamido)phenyl)carbamoyl)-2-methylphenyl) quinazolin-2-yl)amino)-2-methoxy-N-(2-(methylsulfonyl)ethyl)benzamide;
4-((6-(5-((5-(tert-butyl)-2-methoxy-3-(methylsulfonamido)phenyl)carbamoyl)-2-methyl-phenyl)quinazolin-2-yl)amino)-2-methoxy-N-(2-(2-methyl-1H-imidazol-1-yl)ethyl)benzamide;
N-(2-(1H-imidazol-4-yl)ethyl)-4-((6-(5-((5-(tert-butyl)-2-methoxy-3-(methylsulfonamido)-phenyl)carbamoyl)-2-methylphenyl)quinazolin-2-yl)amino)-2-methoxybenzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(2-(pyridin-3-yl)ethyl)benzamide;
4-((6-(5-((5-(tert-butyl)-2-methoxy-3-(methylsulfonamido)phenyl)carbamoyl)-2-methylphenyl)-quinazolin-2-yl)amino)-N-(2-(4-hydroxypiperidin-1-yl)ethyl)-2-methoxybenzamide;
4-((6-(5-((5-(tert-butyl)-2-methoxy-3-(methylsulfonamido)phenyl)carbamoyl)-2-methylphenyl)-quinazolin-2-yl)amino)-2-methoxy-N-((1-methylpiperidin-4-yl)methyl)benzamide;
4-((6-(5-((5-(tert-butyl)-2-methoxy-3-(methylsulfonamido)phenyl)carbamoyl)-2-methylphenyl)-quinazolin-2-yl)amino)-N-(2-(4-hydroxy-1-methylpiperidin-4-yl)ethyl)-2-methoxybenzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(2-(pyrrolidin-1-yl)ethyl)benzamide;
4-((6-(5-((5-(tert-butyl)-2-methoxy-3-(methylsulfonamido)phenyl)carbamoyl)-2-methylphenyl)-quinazolin-2-yl)amino)-2-methoxy-N-(2-(3,3,4-trimethylpiperazin-1-yl)ethyl)benzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(2-morpholinoethyl)benzamide;
4-((6-(5-((5-(tert-butyl)-2-methoxy-3-(methylsulfonamido)phenyl)carbamoyl)-2-methylphenyl)quinazolin-2-yl)amino)-N-(1,1-dioxidotetrahydro-2H-thiopyran-4-yl)-2-methoxybenzamide;
[4-[[6-[5-[[5-tert-butyl-3-(methanesulfonamido)-2-methoxy-phenyl]carbamoyl]-2-methyl-phenyl]quinazolin-2-yl]amino]-2-methoxy-phenyl]-methyl-phosphinic acid;
4-[[6-[5-[[5-tert-butyl-3-(methanesulfonamido)-2-methoxy-phenyl]carbamoyl]-2-methyl-phenyl]quinazolin-2-yl]amino]-2,6-dimethoxy-N-[2-(4-methylpiperazin-1-yl)ethyl]benzamide;
4-[[6-[5-[[5-tert-butyl-3-(methanesulfonamido)-2-methoxy-phenyl]carbamoyl]-2-methyl-phenyl]quinazolin-2-yl]amino]-N-(2-dimethylaminoethyl)-2,6-dimethoxy-benzamide;
2-[5-tert-butyl-2-methoxy-3-[[3-[2-[3-methoxy-4-[2-(1-oxo-1,4-thiazinan-4-yl)ethylcarbamoyl]-anilino]quinazolin-6-yl]-4-methyl-benzoyl]amino]-N-methylsulfonyl-anilino]ethyl dihydrogen phosphate;
2-[[4-[[6-[5-[[5-tert-butyl-3-(methanesulfonamido)-2-methoxy-phenyl]carbamoyl]-2-methyl-phenyl]quinazolin-2-yl]amino]-2,6-dimethoxy-benzoyl]amino]ethyl dihydrogen phosphate;
2-[2-[2-[[4-[[6-[5-[[5-tert-butyl-3-(methanesulfonamido)-2-methoxy-phenyl]carbamoyl]-2-methyl-phenyl]quinazolin-2-yl]amino]-2,6-dimethoxy-benzoyl]amino]ethoxy]ethoxy]ethyl dihydrogen phosphate; and
[2-[5-tert-butyl-2-methoxy-3-[[3-[2-[3-methoxy-4-[2-(1-oxo-1,4-thiazinan-4-yl)ethylcarbamoyl]anilino]quinazolin-6-yl]-4-methyl-benzoyl]amino]anilino]-2-oxo-ethyl]dihydrogen phosphate,
   or a pharmaceutically acceptable salt, solvate or isotopic derivative thereof.

Examples of salts of compounds of formula Ia, Ib, Ic, Id or Ie include all pharmaceutically acceptable salts, such as, without limitation, acid addition salts of strong mineral acids such as HCl, H₂SO₄ and HBr salts (e.g. HCl or HBr salts) and addition salts of strong organic acids such as methanesulfonic acid.

References herein to a compound of the invention (a compounds of formula Ia, Ib, Ic, Id or Ie) are intended to include references to the compound and to all pharmaceutically acceptable salts, solvates and/or tautomers of said compound, unless the context specifically indicates otherwise. In this respect, solvates that may be mentioned include hydrates.

The compounds of the invention (compounds of formula Ia, Ib, Ic, Id or Ie) are p38 MAP kinase inhibitors (especially of the alpha subtype) and are therefore useful in medicine, in particular for the treatment of inflammatory diseases. Further aspects of the invention that may be mentioned therefore include the following.
(a) A pharmaceutical formulation comprising compound of formula Ia, Ib, Ic, Id or Ie, as hereinbefore defined, or pharmaceutically acceptable salt, solvate or isotopic derivative thereof, in admixture with a pharmaceutically acceptable adjuvant, diluent or carrier.
(b) A combination product comprising
   (A) a compound of formula Ia, Ib, Ic, Id or Ie, as hereinbefore defined, or pharmaceutically acceptable salt, solvate or isotopic derivative thereof, and
   (B) another therapeutic agent,
wherein each of components (A) and (B) is formulated in admixture with a pharmaceutically-acceptable adjuvant, diluent or carrier.
In this aspect of the invention, the combination product may be either a single (combination) pharmaceutical formulation or a kit-of-parts.
Thus, this aspect of the invention encompasses a pharmaceutical formulation including a compound of formula Ia, Ib, Ic, Id or Ie, as hereinbefore defined, or pharmaceutically acceptable salt, solvate or isotopic derivative thereof, and another therapeutic agent, in admixture with a pharmaceutically acceptable adjuvant, diluent or carrier (which formulation is hereinafter referred to as a “combined preparation”).
It also encompasses a kit of parts comprising components:
   (i) a pharmaceutical formulation including a compound of formula Ia, Ib, Ic, Id or Ie, as hereinbefore defined, or pharmaceutically acceptable salt, solvate or isotopic derivative thereof, in admixture with a pharmaceutically acceptable adjuvant, diluent or carrier; and
   (ii) a pharmaceutical formulation including another therapeutic agent, in admixture with a pharmaceutically-acceptable adjuvant, diluent or carrier,
which components (i) and (ii) are each provided in a form that is suitable for administration in conjunction with the other.
Component (i) of the kit of parts is thus component (A) above in admixture with a pharmaceutically acceptable adjuvant, diluent or carrier. Similarly, component (ii) is component (B) above in admixture with a pharmaceutically acceptable adjuvant, diluent or carrier.
(c) A process for preparing the pharmaceutical formulation of aspect (a) above, said process comprising the step of admixing the compound of formula Ia, Ib, Ic, Id or Ie, as hereinbefore defined, or pharmaceutically acceptable salt, solvate or isotopic derivative thereof, with a pharmaceutically acceptable adjuvant, diluent or carrier.
Embodiments of this aspect of the invention that may be mentioned include those in which the pharmaceutically acceptable adjuvant, diluent or carrier is a topically acceptable adjuvant, diluent or carrier (and/or wherein the process is for preparing a topical pharmaceutical formulation, i.e. a pharmaceutical formulation that is adapted for topical administration).
(d) A compound of formula Ia, Ib, Ic, Id or Ie, as hereinbefore defined, or pharmaceutically acceptable salt, solvate or isotopic derivative thereof, for use in medicine (or for use as a medicament or as a pharmaceutical).
(e) A compound of formula Ia, Ib, Ic, Id or Ie, as hereinbefore defined, or pharmaceutically acceptable salt, solvate or isotopic derivative thereof, or a pharmaceutical formulation or combination product as defined in connection with aspect (a) or (b) of the invention, for use in the treatment or prevention of an inflammatory disease.
(f) The use of
   a compound of formula Ia, Ib, Ic, Id or Ie, as hereinbefore defined, or pharmaceutically acceptable salt, solvate or isotopic derivative thereof, or
   a pharmaceutical formulation or combination product as defined in connection with aspect (a) or (b) of the invention,
for the preparation of a medicament for the treatment or prevention of an inflammatory disease.
(g) A method of treating or preventing an inflammatory disease, said method comprising administering to a subject an effective amount of
   a compound of formula Ia, Ib, Ic, Id or Ie, as hereinbefore defined, or pharmaceutically acceptable salt, solvate or isotopic derivative thereof, or
   a pharmaceutical formulation or combination product as defined in connection with aspect (a) or (b) of the invention.
(h) A method of sensitizing a subject to the anti-inflammatory effects of a corticosteroid, said method comprising administering to the subject an effective amount of
   a compound of formula Ia, Ib, Ic, Id or Ie, as hereinbefore defined, or pharmaceutically acceptable salt, solvate or isotopic derivative thereof, or
   a pharmaceutical formulation or combination product as defined in connection with aspect (a) or (b) of the invention.
Embodiments of this aspect of the invention that may be mentioned include those in which the subject is one who has become refractory to the anti-inflammatory effects of a corticosteroid.

References herein to “preventing an inflammatory disease” include references to preventing (or reducing the likelihood of) the recurrence of an inflammatory disease in a subject who has previously suffered from such a disease (e.g. a subject who has previously received treatment for that disease, for example treatment according to the method described in (g) above).

Thus, still further aspects of the invention that may be mentioned include the following.
(i) A compound of formula Ia, Ib, Ic, Id or Ie, as hereinbefore defined, or pharmaceutically acceptable salt, solvate or isotopic derivative thereof, or a pharmaceutical formulation or combination product as defined in connection with aspect (a) or (b) of the invention, for use in reducing the likelihood of the recurrence of an inflammatory disease in a subject who has previously received treatment for that disease (e.g. treatment with a compound of formula Ia, Ib, Ic, Id or Ie, as hereinbefore defined, or pharmaceutically acceptable salt, solvate or isotopic derivative thereof, or a pharmaceutical formulation or combination product as defined in connection with aspect (a) or (b) of the invention).
(j) The use of
   a compound of formula Ia, Ib, Ic, Id or Ie, as hereinbefore defined, or pharmaceutically acceptable salt, solvate or isotopic derivative thereof, or
   a pharmaceutical formulation or combination product as defined in connection with aspect (a) or (b) of the invention,
for the preparation of a medicament for reducing the likelihood of the recurrence of an inflammatory disease in a subject who has previously received treatment for that disease (e.g. treatment with a compound of formula Ia, Ib, Ic, Id or Ie, as hereinbefore defined, or pharmaceutically acceptable salt, solvate or isotopic derivative thereof, or a pharmaceutical formulation or combination product as defined in connection with aspect (a) or (b) of the invention).
(k) A method of reducing the likelihood of the recurrence of an inflammatory disease in a subject who has previously received treatment for that disease (e.g. treatment with a compound of formula Ia, Ib, Ic, Id or Ie, as hereinbefore defined, or pharmaceutically acceptable salt, solvate or isotopic derivative thereof, or a pharmaceutical formulation or combination product as defined in connection with aspect (a) or (b) of the invention), said method comprising administering to said subject an effective amount of
   a compound of formula Ia, Ib, Ic, Id or Ie, as hereinbefore defined, or pharmaceutically acceptable salt, solvate or isotopic derivative thereof, or
   a pharmaceutical formulation or combination product as defined in connection with aspect (a) or (b) of the invention.

### Formulations

In relation to aspects (a) and (b) above, diluents and carriers that may be mentioned include those suitable for parenteral, oral, topical, mucosal and rectal administration.

The pharmaceutical formulations and combination products of aspects (a) and (b) above may be prepared e.g. for parenteral, subcutaneous, intramuscular, intravenous, intra-articular, intravitreous, periocular, retrobulbar, subconjunctival, sub-Tenon, topical ocular or peri-articular administration, particularly in the form of liquid solutions, emulsions or suspensions; for oral administration, particularly in the form of tablets or capsules, and especially involving technologies aimed at furnishing colon-targeted drug release (Patel, M. M. Expert Opin. Drug Deliv. 2011, 8 (10), 1247-1258); for topical e.g. pulmonary or intranasal administration, particularly in the form of powders, nasal drops or aerosols and transdermal administration; for topical ocular administration, particularly in the form of solutions, emulsions, suspensions, ointments, implants/inserts, gels, jellies or liposomal microparticle formulations (Ghate, D.; Edelhauser, H. F. Expert Opin. Drug Deliv. 2006, 3 (2), 275-287); for ocular administration, particularly in the form of biodegradable and non-biodegradable implants, liposomes and nanoparticles (Thrimawithana, T. R. et al. Drug Discov. Today 2011, 16 (5/6), 270-277); for mucosal administration e.g. to buccal, sublingual or vaginal mucosa, and for rectal administration e.g. in the form of a suppository or enema.

The pharmaceutical formulations and combination products of aspects (a) and (b) above may conveniently be administered in unit dosage form and may be prepared by any of the methods well-known in the pharmaceutical art, for example as described in Remington's Pharmaceutical Sciences, 17th ed., Mack Publishing Company, Easton, Pa., (1985). Formulations for parenteral administration may contain as excipients sterile water or saline, alkylene glycols such as propylene glycol, polyalkylene glycols such as polyethylene glycol, oils of vegetable origin, hydrogenated naphthalenes and the like. Formulations for nasal administration may be solid and may contain excipients, for example, lactose or dextran, or may be aqueous or oily solutions for use in the form of nasal drops or metered sprays. For buccal administration typical excipients include sugars, calcium stearate, magnesium stearate, pregelatinised starch, and the like.

Pharmaceutical formulations and combination products suitable for oral administration may comprise one or more physiologically compatible carriers and/or excipients and may be in solid or liquid form. Tablets and capsules may be prepared with binding agents, for example, syrup, acacia, gelatin, sorbitol, tragacanth, or poly-vinylpyrrolidone; fillers, such as lactose, sucrose, corn starch, calcium phosphate, sorbitol, or glycine; lubricants, such as magnesium stearate, talc, polyethylene glycol, or silica; and surfactants, such as sodium lauryl sulfate. Liquid compositions may contain conventional additives such as suspending agents, for example sorbitol syrup, methyl cellulose, sugar syrup, gelatin, carboxymethyl-cellulose, or edible fats; emulsifying agents such as lecithin, or acacia; vegetable oils such as almond oil, coconut oil, cod liver oil, or peanut oil; preservatives such as butylated hydroxyanisole (BHA) and butylated hydroxytoluene (BHT). Liquid compositions may be encapsulated in, for example, gelatin to provide a unit dosage form.

Solid oral dosage forms include tablets, two-piece hard shell capsules and soft elastic gelatin (SEG) capsules. Such two-piece hard shell capsules may be made from, for example, gelatin or hydroxylpropyl methylcellulose (HPMC).

A dry shell formulation typically comprises of about 40% to 60% w/w concentration of gelatin, about a 20% to 30% concentration of plasticizer (such as glycerin, sorbitol or propylene glycol) and about a 30% to 40% concentration of water. Other materials such as preservatives, dyes, opacifiers and flavours also may be present. The liquid fill material comprises a solid drug that has been dissolved, solubilized or dispersed (with suspending agents such as beeswax, hydrogenated castor oil or polyethylene glycol 4000) or a liquid drug in vehicles or combinations of vehicles such as mineral oil, vegetable oils, triglycerides, glycols, polyols and surface-active agents.

A compound of the invention may be administered topically (e.g. to the lung, eye or intestines). Thus, embodiments of aspects (a) and (b) above that may be mentioned include pharmaceutical formulations and combination products that are adapted for topical administration. Such formulations include those in which the excipients (including any adjuvant, diluent and/or carrier) are topically acceptable.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC). Suitable CFC propellants include trichloromonofluoromethane (propellant 11), dichlorotetrafluoro methane (propellant 114), and dichlorodifluoromethane (propellant 12). Suitable HFC propellants include tetrafluoroethane (HFC-134a) and heptafluoropropane (HFC-227). The propellant typically comprises 40% to 99.5% e.g. 40% to 90% by weight of the total inhalation composition. The formulation may comprise excipients including co-solvents (e.g. ethanol) and surfactants (e.g. lecithin, sorbitan trioleate and the like). Other possible excipients include polyethylene glycol, polyvinylpyrrolidone, glycerine and the like. Aerosol formulations are packaged in canisters and a suitable dose is delivered by means of a metering valve (e.g. as supplied by Bespak, Valois or 3M or alternatively by Aptar, Coster or Vari).

Topical administration to the lung may also be achieved by use of a non-pressurised formulation such as an aqueous solution or suspension. This may be administered by means of a nebuliser e.g. one that can be hand-held and portable or for home or hospital use (ie non-portable). The formulation may comprise excipients such as water, buffers, tonicity adjusting agents, pH adjusting agents, surfactants and co-solvents. Suspension liquid and aerosol formulations (whether pressurised or unpressurised) will typically contain the compound of the invention in finely divided form, for example with a D₅₀ of 0.5-10 μm e.g. around 1-5 μm. Particle size distributions may be represented using D₁₀, D₅₀ and D₉₀ values. The D₅₀ median value of particle size distributions is defined as the particle size in microns that divides the distribution in half. The measurement derived from laser diffraction is more accurately described as a volume distribution, and consequently the D₅₀ value obtained using this procedure is more meaningfully referred to as a Dv₅₀ value (median for a volume distribution). As used herein Dv values refer to particle size distributions measured using laser diffraction. Similarly, D₁₀ and D₉₀ values, used in the context of laser diffraction, are taken to mean Dv₁₀ and Dv₉₀ values and refer to the particle size whereby 10% of the distribution lies below the D₁₀ value, and 90% of the distribution lies below the D₉₀ value, respectively.

Topical administration to the lung may also be achieved by use of a dry-powder formulation. A dry powder formulation will contain the compound of the disclosure in finely divided form, typically with a mass mean aerodynamic diameter (MMAD) of 1-10 μm or a D₅₀ of 0.5-10 μm e.g. around 1-5 μm. Powders of the compound of the invention in finely divided form may be prepared by a micronization process or similar size reduction process. Micronization may be performed using a jet mill such as those manufactured by Hosokawa Alpine. The resultant particle size distribution may be measured using laser diffraction (e.g. with a Malvern Mastersizer 2000S instrument). The formulation will typically contain a topically acceptable diluent such as lactose glucose or mannitol (preferably lactose), usually of large particle size e.g. an MMAD of 50 μm or more, e.g. 100 μm or more or a D₅₀ of 40-150 μm. As used herein, the term “lactose” refers to a lactose-containing component, including α-lactose monohydrate, β-lactose monohydrate, α-lactose anhydrous, β-lactose anhydrous and amorphous lactose. Lactose components may be processed by micronization, sieving, milling, compression, agglomeration or spray drying. Commercially available forms of lactose in various forms are also encompassed, for example Lactohale® (inhalation grade lactose; DFE Pharma), InhaLac®70 (sieved lactose for dry powder inhaler; Meggle), Pharmatose® (DFE Pharma) and Respitose® (sieved inhalation grade lactose; DFE Pharma) products. In one embodiment, the lactose component is selected from the group consisting of α-lactose monohydrate, α-lactose anhydrous and amorphous lactose. Preferably, the lactose is α-lactose monohydrate.

Dry powder formulations may also contain other excipients such as sodium stearate, calcium stearate or magnesium stearate.

A dry powder formulation is typically delivered using a dry powder inhaler (DPI) device. Examples of dry powder delivery systems include SPINHALER, DISKHALER, TURBOHALER, DISKUS and CLICKHALER. Further examples of dry powder delivery systems include ECLIPSE, NEXT, ROTAHALER, HANDIHALER, AEROLISER, CYCLOHALER, BREEZHALER/NEOHALER, MONODOSE, FLOWCAPS, TWINCAPS, X-CAPS, TURBOSPIN, ELPENHALER, MIATHALER, TWISTHALER, NOVOLIZER, PRESSAIR, ELLIPTA, ORIEL dry powder inhaler, MICRODOSE, PULVINAL, EASYHALER, ULTRAHALER, TAIFUN, PULMOJET, OMNIHALER, GYROHALER, TAPER, CONIX, XCELOVAIR and PROHALER.

In one embodiment a compound of the present invention is provided in a micronized dry powder formulation, for example further comprising lactose of a suitable grade optionally together with magnesium stearate, filled into a single dose device such as AEROLISER or filed into a multi dose device such as DISKUS.

The compounds of the present invention may also be administered rectally, for example in the form of suppositories or enemas, which include aqueous or oily solutions as well as suspensions and emulsions. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the inhibitor will be about 0.0001 to less than 4.0% (w/w).

Preferably, for topical ocular administration, the compositions administered according to the present invention will be formulated as solutions, suspensions, emulsions and other dosage forms. Aqueous solutions are generally preferred, based on ease of formulation, as well as a patient's ability to administer such compositions easily by means of instilling one to two drops of the solutions in the affected eyes. However, the compositions may also be suspensions, viscous or semi-viscous gels, or other types of solid or semi-solid compositions. Suspensions may be preferred for compounds that are sparingly soluble in water.

The compositions administered according to the present invention may also include various other ingredients, including, but not limited to, tonicity agents, buffers, surfactants, stabilizing polymer, preservatives, co-solvents and viscosity building agents. Preferred pharmaceutical compositions of the present invention include the inhibitor with a tonicity agent and a buffer. The pharmaceutical compositions of the present invention may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 5% w/w (e.g. 2 to 4% w/w). Preferred tonicity agents of the invention include the simple sugars or the sugar alcohols, such as D-mannitol.

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7, or a target pH of 6.5 to 7.6.

Surfactants may optionally be employed to deliver higher concentrations of inhibitor. The surfactants function to solubilise the inhibitor and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of the present invention are demulcents which function as a stabilising polymer. The stabilizing polymer should be an ionic/charged example with precedence for topical ocular use, more specifically, a polymer that carries negative charge on its surface that can exhibit a zeta-potential of (−)10-50 mV for physical stability and capable of making a dispersion in water (i.e. water soluble). A preferred stabilising polymer of the invention would be polyelectrolyte, or polyectrolytes if more than one, from the family of cross-linked polyacrylates, such as carbomers and Pemulen™, specifically Carbomer 974p (polyacrylic acid), at 0.1-0.5% w/w.

Other compounds may also be added to the ophthalmic compositions of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

The medical practitioner, or other skilled person, will be able to determine a suitable dosage for the compounds of the invention, and hence the amount of the compound of the invention that should be included in any particular pharmaceutical formulation (whether in unit dosage form or otherwise).

Embodiments of the invention that may be mentioned in connection with the combination products described at (b) above include those in which the other therapeutic agent is one or more therapeutic agents that are known by those skilled in the art to be suitable for treating inflammatory diseases (e.g. the specific diseases mentioned below).

For example, for the treatment of respiratory disorders (such as COPD or asthma), the other therapeutic agent is one or more agents selected from the list comprising:
steroids (e.g. budesonide, beclomethasone dipropionate, fluticasone propionate, mometasone furoate, fluticasone furoate; a further example is ciclesonide);
beta agonists, particularly beta2 agonists (e.g. terbutaline, salbutamol, salmeterol, formoterol; further examples are vilanterol, olodaterol, reproterol and fenoterol); and
xanthines (e.g. theophylline).

For example, for the treatment of respiratory disorders (such as COPD or asthma), the other therapeutic agent is one or more agents selected from the list comprising:
muscarinic antagonists (e.g. tiotropium, umeclidinium, glycopyrronium, aclidinium and daratropium, any of these for example as the bromide salt); and
phosphodiesterase inhibitors.

Further, for the treatment of gastrointestinal disorders (such as Crohn's disease or ulcerative colitis), the other therapeutic agent may be, for example, one or more agents selected from the list comprising:
5-aminosalicylic acid, or a prodrug thereof (such as sulfasalazine, olsalazine or balsalazide);
corticosteroids (e.g. prednisolone, methylprednisolone, or budesonide);
immunosuppressants (e.g. cyclosporin, tacrolimus, methotrexate, azathioprine or 6-mercaptopurine);
anti-TNFα antibodies (e.g. infliximab, adalimumab, certolizumab pegol or golimumab);
anti-IL12/IL23 antibodies (e.g. ustekinumab) or small molecule IL12/IL23 inhibitors (e.g. apilimod);
Anti-α4β7 antibodies (e.g. vedolizumab);
toll-like receptor (TLR) blockers (e.g. BL-7040; Avecia (Cambridge, UK));
MAdCAM-1 blockers (e.g. PF-00547659);
antibodies against the cell adhesion molecule α4-integrin (e.g. natalizumab);
antibodies against the IL2 receptor a subunit (e.g. daclizumab or basiliximab);
anti-Smad7 antibodies (e.g. mongersen (GED0301; all-P-ambo-2′-deoxy-P-thioguanylyl-(3′→5′)-P-thiothymidylyl-(3′→5′)-2′-deoxy-5-methyl-P-thiocytidylyl-(3′→5′)-2′-deoxy-P-thioguanylyl-(3′→5′)-2′-deoxy-P-thiocytidylyl-(3′→5′)-2′-deoxy-P-thiocytidylyl-(3′→5′)-2′-deoxy-P-thiocytidylyl-(3′→5′)-2′-deoxy-P-thiocytidylyl-(3′→5′)-P-thiothymidylyl-(3′→5′)-P-thiothymidylyl-(3′→5′)-2′-deoxy-Pthiocytidylyl-(3′→5′)-P-thiothymidylyl-(3′→5′)-2′-deoxy-P-thiocytidylyl-(3′→5′)-2′-deoxy-P-thiocytidylyl-(3′→5′)-2′-deoxy-P-thiocytidylyl-(3′→5′)-2′-deoxy-5-methyl-P-thiocytidylyl-(3′→5′)-2′-deoxy-Pthioguanylyl-(3′→5′)-2′-deoxy-P-thiocytidylyl-(3′→5′)-2′-deoxy-Pthioadenylyl-(3′→5′)-2′-deoxy-P-thioguanylyl-(3′-5′)-2′-deoxycytidine));
sphingosine 1-phosphate receptor 1 (S1P1) modulators (e.g. ozanimod (S)-5-(3-(1-((2-hydroxyethyl)amino)-2,3-dihydro-1H-inden-4-yl)-1,2,4-oxadiazol-5-yl)-2-isopropoxybenzonitrile), amiselimod (MT1303; 2-amino-2-{2-[4-(heptyloxy)-3-(trifluoromethyl)phenyl]ethyl}propane-1,3-diol) or APD334 (2-[7-[4-cyclopentyl-3-(trifluoromethyl)benzyloxy]-1,2,3,4-tetrahydrocyclopenta[b]indol-3(R)-yl]acetic acid));
JAK inhibitors (e.g. tofacitinib, baricitinib(1-(ethylsulfonyl)-3-[4-(7h-pyrrolo[2,3-d]pyrimidin-4-yl)-1 h-pyrazol-1-yl]-3-azetidineacetonitrile), filgotinib (N-[5-[4-[(1,1-dioxo-1,4-thiazinan-4-yl)methyl]phenyl]-[1,2,4]triazolo[1,5-a]pyridin-2-yl]cyclopropanecarboxamide), peficitinib (4-(((1R,2r,3S,5s,7s)-5-hydroxyadamantan-2-yl)amino)-1H-pyrrolo[2,3-b]pyridine-5-carboxamide) or R348 (see, for example, US 2014/0206708));
STAT3 inhibitors (e.g. TAK-114; (3E)-1-methyl-3-(2-oxo-1H-indol-3-ylidene)indol-2-one);
receptor-interacting protein-1 (RIP1) kinase inhibitors (e.g. GSK2982772);
Syk inhibitors and prodrugs thereof (e.g. fostamatinib and R-406);
Phosphodiesterase-4 inhibitors (e.g. tetomilast);
HMPL-004;
probiotics;
microbiome modulators (e.g. SGM1019);
Dersalazine;
semapimod/CPSI-2364; and
protein kinase C inhibitors (e.g. AEB-071)
   (e.g. for the treatment of gastrointestinal disorders (such as Crohn's disease or ulcerative colitis), the other therapeutic agent may be, for example, one or more agents selected from the list comprising:
5-aminosalicylic acid, or a prodrug thereof (such as sulfasalazine, olsalazine or balsalazide);
corticosteroids (e.g. prednisolone, methylprednisolone, or budesonide);
immunosuppressants (e.g. cyclosporin, tacrolimus, methotrexate, azathioprine or 6-mercaptopurine);
anti-TNFα antibodies (e.g. infliximab, adalimumab, certolizumab pegol or golimumab);
anti-IL12/IL23 antibodies (e.g. ustekinumab) or small molecule IL12/IL23 inhibitors (e.g. apilimod);
Anti-α4β7 antibodies (e.g. vedolizumab);
MAdCAM-1 blockers (e.g. PF-00547659);
antibodies against the cell adhesion molecule α4-integrin (e.g. natalizumab);
antibodies against the IL2 receptor a subunit (e.g. daclizumab or basiliximab);
JAK3 inhibitors (e.g. tofacitinib or R348);
Syk inhibitors and prodrugs thereof (e.g. fostamatinib and R-406);
Phosphodiesterase-4 inhibitors (e.g. tetomilast);
HMPL-004;
probiotics;
Dersalazine;
semapimod/CPSI-2364; and
protein kinase C inhibitors (e.g. AEB-071)).

For the treatment of eye disorders (such as uveitis), the other therapeutic agent may be, for example, one or more agents selected from the list comprising:
corticosteroids (e.g. dexamethasone, prednisolone, triamcinolone acetonide, difluprednate or fluocinolone acetonide);
immunosuppressants (e.g. cyclosporin, voclosporin, azathioprine, methotrexate, mycophenolate mofetil or tacrolimus);
anti-TNFα antibodies (e.g., infliximab, adalimumab, certolizumab pegol, ESBA-105 or golimumab);
anti-IL-17A antibodies (e.g., secukinumab);
mTOR inhibitors (e.g., sirolimus);
VGX-1027;
adenosine A3 receptor agonists (e.g., CF-101);
lifitegrast;
IL1 blockers (e.g. EBI-005; Hou et al. PNAS 2013, 110(10), 3913-3918);
RGN-259 (Thymosin 34);
SI-614;
OTX-101;
JNK inhibitors (e.g. XG-104);
MAP kinase signalling inhibitors (e.g. DA-6034; {[2-(3,4-dimethoxyphenyl)-5-methoxy-4-oxochromen-7-yl]oxy}acetic acid);
mucin stimulators (e.g. rebamipide; 2-[(4-chlorobenzoyl)amino]-3-(2-oxo-1H-quinolin-4-yl)propanoic acid);
MIM-D3 (see, for example, US 2013/0345395);
JAK inhibitors (e.g. tofacitinib, baricitinib (1-(ethylsulfonyl)-3-[4-(7h-pyrrolo[2,3-d]pyrimidin-4-yl)-1 h-pyrazol-1-yl]-3-azetidineacetonitrile), filgotinib (N-[5-[4-[(1,1-dioxo-1,4-thiazinan-4-yl)methyl]phenyl]-[1,2,4]triazolo[1,5-a]pyridin-2-yl]cyclopropanecarboxamide), peficitinib (4-(((1R,2r,3S,5s,7s)-5-hydroxyadamantan-2-yl)amino)-1H-pyrrolo[2,3-b]pyridine-5-carboxamide) or R348 (see, for example, US 2014/0206708)); and
protein kinase C inhibitors (e.g. AEB-071).
   (e.g. for the treatment of eye disorders (such as uveitis and keratoconjunctivitis sicca (dry eye)), the other therapeutic agent may be, for example, one or more agents selected from the list comprising:
corticosteroids (e.g. dexamethasone, prednisolone, triamcinolone acetonide, difluprednate or fluocinolone acetonide);
glucocorticoid agonists (e.g. mapracorat);
immunosuppressants (e.g. cyclosporin, voclosporin, azathioprine, methotrexate, mycophenolate mofetil or tacrolimus);
anti-TNFα antibodies (e.g. infliximab, adalimumab, certolizumab pegol, ESBA-105 or golimumab);
anti-IL-17A antibodies (e.g. secukinumab);
mTOR inhibitors (e.g. sirolimus);
VGX-1027;
JAK3 inhibitors (e.g. tofacitinib or R348); and
protein kinase C inhibitors (e.g. AEB-071)).

### Medical Uses

The compounds of the invention may be used as monotherapies for inflammatory diseases, or in combination therapies for such diseases.

Thus, embodiments of aspects (e) to (g) above that may be mentioned include those in which the compound of formula Ia, Ib, Ic, Id or Ie (or pharmaceutically acceptable salt, solvate or isotopic derivative thereof) is the sole pharmacologically active ingredient utilised in the treatment.

However, in other embodiments of aspects (e) to (g) above, the compound of formula Ia, Ib, Ic, Id or Ie (or pharmaceutically acceptable salt, solvate or isotopic derivative thereof) is administered to a subject who is also administered one or more other therapeutic agents (e.g. wherein the one or more other therapeutic agents are as defined above in connection with combination products).

When used herein, the term “inflammatory disease” specifically includes references to any one or more of the following:
(i) lung diseases or disorders having an inflammatory component, such as cystic fibrosis, pulmonary hypertension, lung sarcoidosis, idiopathic pulmonary fibrosis or, particularly, COPD (including chronic bronchitis and emphysema), asthma or paediatric asthma;
(ii) skin diseases or disorders having an inflammatory component, such as atopic dermatitis, allergic dermatitis, contact dermatitis or psoriasis;
(iii) nasal diseases or disorders having an inflammatory component, such as allergic rhinitis, rhinitis or sinusitis;
(iv) eye diseases or disorders having an inflammatory component, such as conjunctivitis, allergic conjunctivitis, keratoconjunctivitis sicca (dry eye, also known as xerophthalmia), glaucoma, diabetic retinopathy, macular oedema (including diabetic macular oedema), central retinal vein occlusion (CRVO), dry and/or wet age related macular degeneration (AMD), post-operative cataract inflammation, or, particularly, uveitis (including posterior, anterior and pan uveitis), corneal graft and limbal cell transplant rejection; and
(v) gastrointestinal diseases or disorders having an inflammatory component, such as gluten sensitive enteropathy (coeliac disease), eosinophilic esophagitis, intestinal graft versus host disease or, particularly, Crohn's disease or ulcerative colitis.

References herein to diseases having an inflammatory component include references to diseases that involve inflammation, whether or not there are other (non-inflammatory) symptoms or consequences of the disease.

According to a further aspect of the invention there is provided a process for the preparation of a compound of formula I which process comprises:

(a) reaction of a compound of formula II,

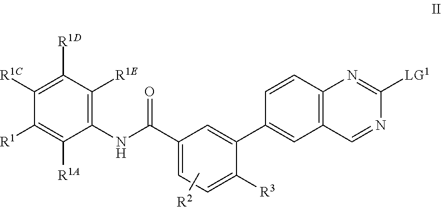

wherein LG¹ represents a suitable leaving group (e.g. a halo group such as fluoro, chloro or bromo, or a —S(O)₀₋₂—CH₃ group) and R¹, R^{1A}, R^{1C}, R^{1D}, R^{1E}, R² and R³ are as hereinbefore defined with a compound of formula Ill,

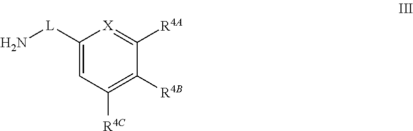

wherein L, X, R^{4A}, R^{4B} and R^{4C} are as hereinbefore defined, for example under conditions known to those skilled in the art (e.g. reaction in the presence of an aprotic organic solvent, such as DMF or 1,4-dioxane, and a catalyst, such as p-toluene sulfonic acid or a Pd(0) complex (e.g. a complex formed between Pd₂(dba)₃ and BINAP, optionally in the presence of a base, such as an alkali metal carbonate), or reaction at elevated temperature (e.g. 60 to 100° C.) in the presence of an aprotic organic solvent such 1,4-dioxane and a tertiary amine base, such as triethylamine or diisopropylethylamine);
(b) reaction of a compound of formula IV,

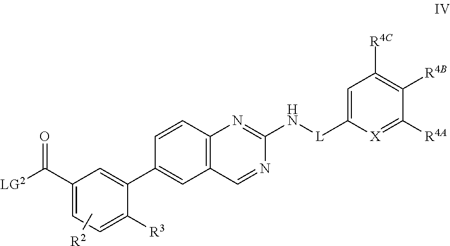

wherein LG² represents a suitable leaving group (e.g. halo, OH or O—C₁₋₄ alkyl) and R², R³, L, X, R^{4A}, R^{4B} and R^{4C} are as hereinbefore defined, with a compound of formula V,

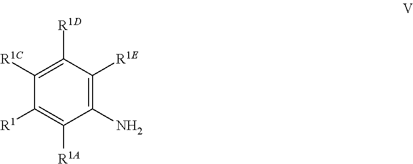

wherein R¹, R^{1A}, R^{1C}, R^{1D} and R^{1E} are as hereinbefore defined, under conditions known to those skilled in the art, for example
when LG² represents halo (e.g. chloro), reaction with the compound of formula (XI) in the presence of a suitable solvent and a base (e.g. triethylamine or N,N-diisopropylethylamine),
when LG² represents OH, reaction in the presence of a suitable solvent, a base (e.g. triethylamine or N,N-diisopropylethylamine) and an amide (peptide) coupling reagent, such as HATU, CDI, N,N′-dicyclohexylcarbodiimide, N,N′-diisopropylcarbodiimide, BOP or PyBOP, optionally in combination with an activated ester-forming agent such as HOBt or 1-hydroxy-7-azabenzotriazole,
when LG² represents OH, conversion of the carboxylic acid to an acid halide (e.g. by reaction with a halogenating agent such as thionyl chloride), followed by reaction with the compound of formula (XI) in the presence of a suitable solvent and a base (e.g. triethylamine or N,N-diisopropylethylamine), or
when LG² represents O—C₁₋₄ alkyl (e.g. OCH₃), reaction in the presence of a trialkylaluminium (e.g. trimethylaluminium) and an aprotic solvent (e.g. THF);
   (c) for compounds of formula I in which R¹ represents -L¹-C(O)NR^{X}R^{Y}, -L²-S(O)₂R^{Y1}, -L³-P(O)R^{Y1}R^{Y2}, -L⁴-S(O)₂NR^{X}R^{Y}, -L⁵-C(O)R^{Y} or -L⁷-C(O)OR^{Y}, in which L¹, L², L³, L⁴, L⁵ and L⁷ represent —N(R^{X2})— or —C(R^{a})(R^{b})—N(R^{X2})—, reaction of a compound of formula VI,

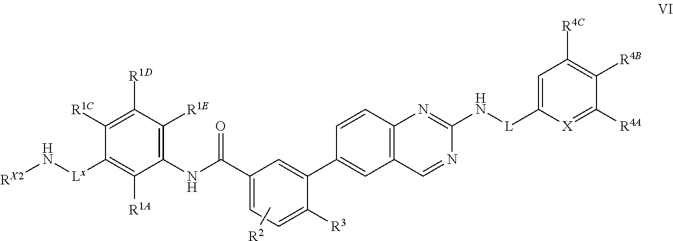

wherein L^{x} represents a direct bond or —C(R^{a})(R^{b})— and R^{X2}, R^{1A}, R^{1C}, R^{1D}, R^{1E}, R², R³, L, X, R^{4A}, R^{4B} and R^{4C} are as hereinbefore defined, with a compound of formula VIIa, VIIb, VIIc, VIId, VIIe or VIIf,

LG³-C(O)NR^{X}R^{Y} VIIa

LG³-S(O)₂R^{Y1} VIIb

LG³-P(O)R^{Y1}R^{Y2} VIIc

LG³-S(O)₂NR^{X}R^{Y} VIId

LG³-C(O)R^{Y} VIIe

LG³-C(O)OR^{Y} VIIf

wherein LG³ represents a suitable leaving group (e.g. halo) and R^{X}, R^{Y}, R^{Y1} and R^{Y2} are as hereinbefore defined, for example under conditions known to those skilled in the art;
(d) for compounds of formula I in which R¹ represents -L¹-C(O)NR^{X}R^{Y} in which L¹ represents a bond or -[C(R^{a})(R^{b})]₁₋₂—, reaction of a compound of formula VIII,

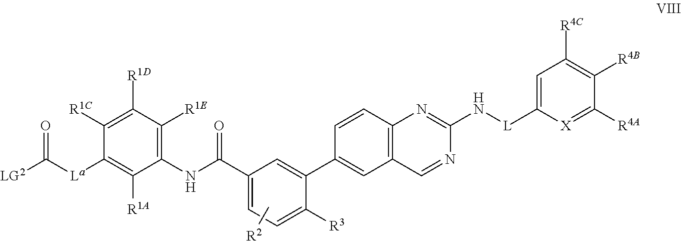

wherein L^{a} represents a bond or —[C(R^{a})(R^{b})]₁₋₂- and R^{1A}, R^{1C}, R^{1D}, R^{1E}, R², R³, L, X, R^{4A}, R^{4B}R^{4C} and LG² are as hereinbefore defined, with a compound of formula IX,

HNR^{X}R^{Y} IX

wherein R^{X} and R^{Y} are as hereinbefore defined, for example under conditions know to those skilled in the art (e.g. the conditions described in (b) above);
(e) for compounds of formula I where
Q¹, Q² or Q³ represents —C(O)N(R⁸)— or —S(O)₂N(R⁸)— or
R^{5a} represents —C₁₋₄-alkylene-CO—N(R^{7b})R^{7c} or —S(O)₂—N(R^{7b})R^{7c}, condensation of a compound of formula VIIIa—

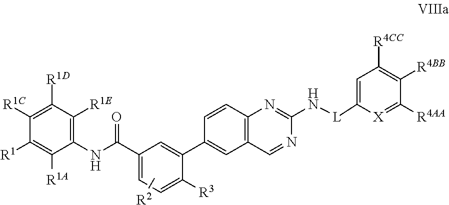

wherein one of R^{4AA}, R^{4BB} and R^{4CC} represents —[C₁₋₄-alkylene]₀₋₁-C(O)LG² or —S(O)₂LG² and each of the other two of R^{4AA}, R^{4BB} and R^{4CC} independently represents R^{5b} and R¹, R^{1A}, R^{1C}, R^{1D}, R^{1E}, R², R³, R^{5b}, L, X and LG² are as hereinbefore defined, with an amine of formula IXa, IXb, IXc or IXd,

HNR⁸—C(R^{6c})(R^{6d})—[C₁₋₅ alkylene]-R^{6a1} IXa

HNR⁸—[C(R^{6c})(R^{6d})—(CH₂)₀₋₁CH₂—O]₁₋₁₂—CH₂(CH₂)₀₋₁CH₂—R^{6a1} IXb

HNR⁸-J-Het^{X} IXc

HN(R^{7b})R^{7c} IXd

which C₁₋₅ alkylene and Het^{X} groups are optionally substituted as described above, wherein R^{6c}, R^{6d}, R⁸, J and Het^{X} are as hereinbefore defined, and R^{6a1} takes the same definition as R^{6a} above, except that CO₂H is only present in protected form (e.g. as C(O)O—C₁₋₄ alkyl), for example under conditions known to those skilled in the art, such as
when LG² represents OH, reaction in the presence of a tertiary amine base (e.g. a trialkylamine such as triethylamine or diisopropylethylamine or a cyclic amine such as N-methylpyrrolidine or N-methylmorpholine), an amide (peptide) coupling reagent (e.g. T3P, HATU, TBTU, CDI, BOP, PyBOP, HOAt, HOBt or a carbodiimide such as DCC or diisopropylcarbodiimide) and an aprotic organic solvent (e.g. a chlorinated solvent such as DCM, an ester such as ethyl acetate, an amide of dimethylamine such as DMF, a lactam such as NMP, or a mixture of any such solvents), followed, if necessary, by deprotection of R^{6a1} when that group represents C(O)O—C₁₋₄ alkyl or
when LG² represents halo (e.g. chloro), reaction in the presence of a suitable solvent and a base (e.g. triethylamine or N,N-diisopropylethylamine);
   (f) for compounds of formula I in which
R¹ represents —C₁₋₄ alkylene-OP(O)(OH)₂,
R^{X} and/or R^{X1} represents C₁₋₆ alkyl substituted by —OP(O)(OH)₂,
R^{Y}, R^{Y1} and/or R^{Y2} represents C₁₋₆ alkyl, C₃₋₇ cycloalkyl, phenyl, benzyl, Het¹ or Het², which latter six groups are substituted by —OP(O)(OH)₂ and are optionally further substituted by one or more substituents selected from halo, hydroxy, C₁₋₄ alkyl, C₁₋₄ alkoxy, C(O)OH, —N(R^{c})(R^{d}) and Het³, which latter group is optionally substituted by C₁₋₄ alkyl,
R^{X2} represents C₁₋₄ alkyl substituted by —OP(O)(OH)₂,
R^{1A} represents C₁₋₆ alkoxy, C₁₋₆ alkyl, C₂₋₆ alkenyl, C₂₋₆ alkynyl, which latter four groups are substituted by —OP(O)(OH)₂ and are optionally further substituted by one or more substituents selected from C₁₋₂ alkyl, halo, hydroxy, C₁₋₂ alkoxy and —N(R^{c})(R^{d}) or Het⁴,
R^{1D} represents C₂₋₇ alkyl, C₂₋₇ alkenyl, C₂₋₇ alkynyl, C₃₋₇ cycloalkyl, phenyl, Het¹ or Het², which latter seven groups are substituted by —OP(O)(OH)₂ and are optionally further substituted by one or more substituents selected from C₁₋₄ alkyl, halo, cyano, hydroxy and C₁₋₄ alkoxy, or R^{1D} represents trimethylsilyl or trifluoromethyl,
R^{5a} represents —CH₂OP(O)(OH)₂,
R^{5b} represents C₁₋₃ alkoxy or C₁₋₃ alkyl, which latter two groups are substituted by —OP(O)(OH)₂,
R^{6b} represents C₁₋₈ alkyl, C₃₋₈ cycloalkyl, phenyl, Het¹ or Het², which latter five groups are substituted by —OP(O)(OH)₂ and are optionally further substituted by one or more substituents selected from halo, hydroxyl, C₁₋₄ alkyl and C₁₋₄ alkoxy and which Het² group is optionally substituted or further substituted by one or more oxo groups,
R^{7a}, R^{7b} and/or R^{7c} represents —C(R^{7d})(R^{7e})—C₁₋₃ alkylene-OP(O)(OH)₂ or
R^{7a} represents —P(O)(OH)₂,
   reaction of a hydroxy group on a corresponding compound of formula I in which, respectively
R¹ represents —C₁₋₄ alkylene-OH,
R^{X} and/or R^{X1} represents C₁₋₆ alkyl substituted by hydroxy,
R^{Y}, R^{Y1} and/or R^{Y2} represents C₁₋₆ alkyl, C₃₋₇ cycloalkyl, phenyl, benzyl, Het¹ or Het², which latter six groups are substituted by hydroxy and are optionally further substituted by one or more substituents selected from halo, hydroxy, C₁₋₄ alkyl, C₁₋₄ alkoxy, C(O)OH, —N(R^{c})(R^{d}) and Het³, which latter group is optionally substituted by C₁₋₄ alkyl,
R^{X2} represents C₁₋₄ alkyl substituted by hydroxy,
R^{1A} represents C₁₋₆ alkoxy, C₁₋₆ alkyl, C₂₋₆ alkenyl, C₂₋₆ alkynyl, which latter four groups are substituted by hydroxy and are optionally further substituted by one or more substituents selected from C₁₋₂ alkyl, halo, hydroxy, C₁₋₂ alkoxy and —N(R^{c})(R^{d}) or Het⁴,
R^{1D} represents C₂₋₇ alkyl, C₂₋₇ alkenyl, C₂₋₇ alkynyl, C₃₋₇ cycloalkyl, phenyl, Het¹ or Het², which latter seven groups are substituted by hydroxy and are optionally further substituted by one or more substituents selected from C₁₋₄ alkyl, halo, cyano, hydroxy and C₁₋₄ alkoxy, or R^{1D} represents trimethylsilyl or trifluoromethyl,
R^{5a} represents —CH₂OH,
R^{5b} represents C₁₋₃ alkoxy or C₁₋₃ alkyl, which latter two groups are substituted by hydroxy,
R^{6b} represents C₁₋₈ alkyl, C₃₋₈ cycloalkyl, phenyl, Het¹ or Het², which latter five groups are substituted by hydroxy and are optionally further substituted by one or more substituents selected from halo, hydroxyl, C₁₋₄ alkyl and C₁₋₄ alkoxy and which Het² group is optionally substituted or further substituted by one or more oxo groups,
R^{7a}, R^{7b} and/or R^{7c} represents —C(R^{7d})(R^{7e})—C₁₋₃ alkylene-OH or
R^{7a} represents H,
   with di-tert-butyl diethylphosphoramidite (Dunn, D., et al., J. Biol. Chem. 1996, 271, 168-173) or dibenzyl-N,N-diisopropylphosphoramidite (Locher, C. P., et al., WO 2014/014845, 23 Jan. 2014), for example in the presence of an activator (e.g. tetrazole or 5-methyl-1H-tetrazole) and an aprotic organic solvent (e.g. THF or DMF), followed by reaction with an oxidant (e.g. hydrogen peroxide or mCPBA) and then finally removal of the respective tert-butyl or benzyl protecting groups by hydrolysis using an acid (such as trifluoroacetic acid) or hydrogenolysis with a palladium catalyst, (such as Pd/C), optionally in the presence of a base (e.g. sodium hydroxide, sodium bicarbonate or sodium carbonate); or
   (g) deprotection of a protected derivative of a compound of formula I, under conditions known to those skilled in the art, wherein the protected derivative bears a protecting group on an O- or N-atom of the compound of formula I (and, for the avoidance of doubt, a protected derivative of one compound of formula I may or may not represent another compound of formula I).

Compounds of formula III may be prepared according to or by analogy with procedures known to those skilled in the art, for example as described below.
(i) For compounds of formula III in which L represents a direct bond and one of R^{4A}, R^{4B} and R^{4C} represents
   —O—[C(R^{6c})(R^{6d})—(CH₂)₀₋₁CH₂—O]₁₋₁₂—CH₂(CH₂)₀₋₁ CH₂—R^{6a}
   —S—[C(R^{6c})(R^{6d})—(CH₂)₀₋₁CH₂—O]₁₋₁₂—CH₂(CH₂)₀₋₁CH₂—R^{6a}
   —O—C(R^{6c})(R^{6d})—[C₁₋₅ alkylene]-R^{6a} or
   —S—C(R^{6c})(R^{6d})—[C₁₋₅ alkylene]-R^{6a},
reaction of a compound of formula X,

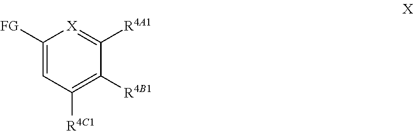

wherein one of R^{4A1}, R^{4B1} and R^{4C1} represents OH or SH, and each of the other two of R^{4A1}, R^{4B1} and R^{4C1} independently represents R^{5b}, FG represents NH₂, N(H)—PG (wherein PG is an amino protecting group) or amino in masked form (e.g. NO₂), and X and R^{5b} are as hereinbefore defined, with a compound of formula XIa or XIb,

LG⁴-[C(R^{6c})(R^{6d})—(CH₂)₀₋₁CH₂—O]₁₋₁₂—CH₂(CH₂)₀₋₁CH₂—R^{6a} XIa

LG⁴-C(R^{6c})(R^{6d})—[C₁₋₅ alkylene]-R^{6a} XIb
wherein LG⁴ represents a suitable leaving group such as halo, (perfluoro)alkanesulfonate or arylsulfonate (e.g. methanesulfonate or p-toluenesulfonate), R^{6e}, R^{6f}, R^{6g} and R^{6h} are as hereinbefore defined, under conditions known to those skilled in the art (e.g. in the presence of an organic solvent and either a suitable base or, in the case of reaction with the compound of formula XVId, a suitable acid, such as trifluoroacetic acid), followed by
   when FG is N(H)—PG, removal of the PG protecting group or
   when FG is NO₂, reduction of NO₂ to NH₂.
(ii) For compounds of formula III in which L represents a direct bond and one of R^{4A}, R^{4B} and R^{4C} represents
   —S(O)₁₋₂—[C(R^{6c})(R^{6d})—(CH₂)₀₋₁CH₂—O]₁₋₁₂—CH₂(CH₂)₀₋₁CH₂—R^{6a},
   —S(O)₁₋₂—C(R^{6c})(R^{6d})—[C₁₋₅ alkylene]-R^{6a},
   —S(O)₁₋₂-Het³ or
   —S(O)₁₋₂—R^{6b},
oxidation of a corresponding compound of formula III in which one of R^{4A}, R^{4B} and R^{4C} represents
   —S—[C(R^{6c})(R^{6d})—(CH₂)₀₋₁CH₂—O]₁₋₁₂—CH₂(CH₂)₀₋₁CH₂—R^{6a},
   —S—C(R^{6c})(R^{6d})—[C₁₋₅ alkylene]-R^{6a},
   —S-Het³ or
   —S—R^{6b},
under conditions known to those skilled in the art (e.g. in the presence of a peracid, such as meta-chloroperbenzoic acid).
(iii) For compounds of formula III in which L represents a direct bond and one of R^{4A}, R^{4B} and R^{4C} represents —S(O)₂R^{6b}, coupling of a compound of formula XII,

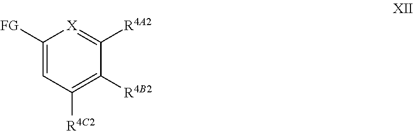

wherein one of R^{4A2}, R^{4B2} and R^{4C2} represents LG⁴, and each of the other two of R^{4A2}, R^{4B2} and R^{4C2} independently represents R^{5b}, and X, R^{5b}, FG and LG⁴ are as hereinbefore defined, with a compound of formula XIII,

M⁺O⁻—S(O)—R^{6b} XIII
wherein M⁺ represents a monovalent metal cation (e.g. an alkali metal cation, such as a potassium cation) and R^{6b} is as hereinbefore defined, under conditions known to those skilled in the art (e.g. in the presence of a Pd(O) catalyst, Cu(I) iodide and a suitable base), followed by
   when FG is N(H)—PG, removal of the PG protecting group or
   when FG is NO₂, reduction of NO₂ to NH₂.
(iv) For compounds of formula III in which L represents a direct bond and one of R^{4A}, R^{4B} and R^{4C} represents -J-P(O)R^{6e}R^{6f}, where J represents a direct bond, coupling of a compound of formula XII, wherein one of R^{4A2}, R^{4B2} and R^{4C2} represents LG⁴, as hereinbefore defined (e.g. halo or trifluoromethanesulfonate), and each of the other two of R^{4A2}, R^{4B2} and R^{4C2} independently represents R^{5b}, wherein X, R^{5b} and FG are as hereinbefore defined, with a compound of formula XIIIa,

HP(O)R^{6e}R^{6f} XIIIa
wherein R^{6e} and R^{6f} are as hereinbefore defined, under conditions known to those skilled in the art, such as in the presence of a palladium-containing catalyst (e.g. Pd(II) acetate and, optionally, a bidentate phosphine ligand such as 4,5-bis(diphenylphosphino)-9,9-dimethylxanthene (Xantphos)), followed by
   when FG is N(H)—PG, removal of the PG protecting group or
   when FG is NO₂, reduction of NO₂ to NH₂.
(v) For compounds of formula III in which L represents a direct bond and one of R^{4A}, R^{4B} and R^{4C} represents
   -Q^{1a}-C(R^{6c})(R^{6d})—[C₁₋₅ alkylene]-R^{6a},
   -Q^{2a}-[C(R^{6c})(R^{6d})—(CH₂)₀₋₁CH₂—O]₁₋₁₂—CH₂(CH₂)₀₋₁CH₂—R^{6a}, or
   -Q^{3a}-J-Het^{X},
where Q^{1a}, Q^{2a} and Q^{3a} represent —C(O)N(R⁸)— or —S(O)₂N(R⁸)—, reaction of a compound of formula XII, wherein one of R^{4A2}, R^{4B2} and R^{4C2} represents —C(O)LG² or —S(O)₂LG² and each of the other two of R^{4A2}, R^{4B2} and R^{4C2} independently represents R^{5b}, wherein X, R^{5b}, LG² and FG are as hereinbefore defined, with an amine of formula IXa, IXb or IXc (for example employing the peptide coupling conditions described in respect of process (e) above), followed by
   when FG¹ represents NH-PG², removal of the PG² protecting group,
   when FG¹ represents NO₂, reduction of NO₂ to NH₂.

Compounds of formula V may be prepared according to or by analogy with procedures known to those skilled in the art, for example by the procedure described below.
(a) For compounds of formula V in which R¹ represents —P(O)R^{Y1}R^{Y2}, cross-coupling of a compound of formula XIV,

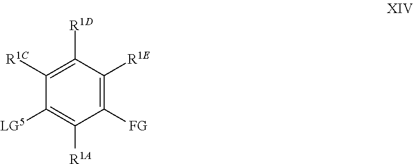

wherein LG⁵ represents a suitable leaving group such as halo (e.g. bromo or iodo) and R^{1A}, R^{1C}, R^{1D}, R^{1E} and FG are as hereinbefore defined and, with a compound of formula

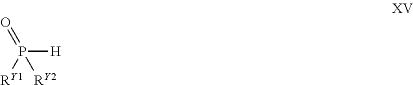

wherein R^{Y1} and R^{Y2} are as hereinbefore defined, for example under conditions known to those skilled in the art, such as in the presence of a palladium-containing catalyst (e.g. Pd(II) acetate, optionally in the presence of a bidentate phosphine ligand such as 4,5-bis(diphenylphosphino)-9,9-dimethylxanthene (Xantphos); see, for example, WO 2009/143389) to furnish the aryl phosphine oxide, followed by
   when FG is N(H)—PG, removal of the PG protecting group or
   when FG is NO₂, reduction of NO₂ to NH₂.
(b) For compounds of formula V in which R¹ represents —[C(R^{a})(R^{b})]₁₋₂—P(O)R^{Y1}R^{Y2}, cross-coupling of a compound of formula XVI,

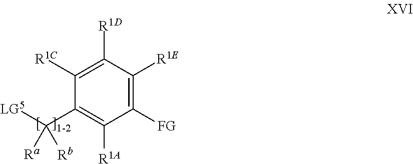

wherein R^{1A}, R^{1C}, R^{1D}, R^{1E}, R^{a}, R^{b}, LG⁶ and FG are as hereinbefore defined, with a compound of formula XV, as hereinbefore defined, for example under conditions known to those skilled in the art, for example, utilising a transition metal, such as a palladium (see, for example, Org. Lett. 2011, 13, 3270-3273 and WO 2009/143389) or nickel (Bioorg. Med. Chem. Lett. 2009, 19, 2053-2058), catalyst to generate a phosphoryl-carbon bond, or, alternatively, the compounds of formula XVI are coupled in an Arbuzov-type reaction (WO 2010/141406; Bioorg. Med. Chem. Lett. 2009, 19, 2053-2058) with compounds XVII,

(R^{Y1})(R^{Y2})P—OC₁₋₄ alkyl XVII
wherein R^{2d} and R^{2e} are as hereinbefore defined, and XXIa are typically made in situ by reaction of the corresponding chlorophosphine (R^{2d})(R^{2e})P—Cl with a C₁₋₄ alkyl alcohol in the presence or a base (e.g. diisopropylethylamine) or with an alkali metal salt of a C₁₋₄ alkyl alcohol, followed by
when FG is N(H)—PG, removal of the PG protecting group or
when FG is NO₂, reduction of NO₂ to NH₂.
(c) For compounds of formula V in which R¹ represents —[C(R^{a})(R^{b})]₀₋₂—C(O)NH₂, hydrolysis of a nitrile of formula XVIII,

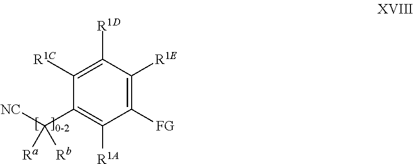

wherein R^{1A}, R^{1C}, R^{1D}, R^{1E}, R^{a}, R^{b} and FG are as hereinbefore defined, for example under conditions known to those skilled in the art, such as hydrolysis with concentrated hydrochloric acid at elevated temperature, e.g., from 30 to 70° C., to the primary amide, followed by
   when FG is N(H)—PG, removal of the PG protecting group or
   when FG is NO₂, reduction of NO₂ to NH₂.
(d) For compounds of formula V in which R¹ represents —[C(R^{a})(R^{b})]₀₋₂—C(O)NR^{X}R^{Y} reaction of a compound of formula XIX,

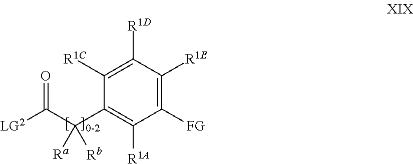

wherein R^{1A}, R^{1C}, R^{1D}, R^{1E}, LG² and FG are as hereinbefore defined, with a compound of formula XX,

HNR^{X}R^{Y} XX
wherein R^{X} and R^{Y} are as hereinbefore defined, for example under conditions known to those skilled in the art (see, for example, process (b) above in relation to the compounds of formula I), followed by
   when FG is N(H)—PG, removal of the PG protecting group or
   when FG is NO₂, reduction of NO₂ to NH₂.
(e) For compounds of formula V in which R¹ represents —S—R^{Y1}, reaction of a compound of formula XXI,

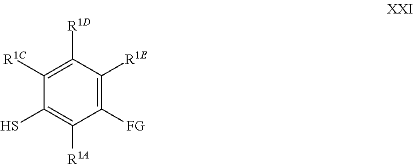

wherein R^{1A}, R^{1C}, R^{1D}, R^{1E} and FG are as hereinbefore defined, with a compound of formula XXII,

LG⁴-R^{Y1} XXII
wherein LG⁴ and R^{Y1} are as hereinbefore defined, for example under basic (potassium carbonate, sodium ethoxide or triethylamine) conditions when R^{Y1} is C₁₋₆ alkyl, C₃₋₇ cycloalkyl, benzyl or Het², or under transition metal-catalysed cross-coupling conditions, such as copper(I) iodide (J. Org. Chem. 2010, 75, 3626-3643) or a palladium-containing catalyst (WO 2007117381, 18 Oct. 2007), when R^{Y1} is phenyl, or Het¹, followed by
   when FG is N(H)—PG, removal of the PG protecting group or
   when FG is NO₂, reduction of NO₂ to NH₂.
(f) For compounds of formula V in which R¹ represents —[C(R^{a})(R^{b})]₁₋₂—S—R^{Y1}, reaction of a compound of formula XVI, as hereinbefore defined, with a compound of formula XXIII, or the sodium salt—prepared by treatment with base such as sodium hydride or sodium hydroxide—thereof

H—S—R^{Y1} XXIII
wherein R^{Y1} is as hereinbefore defined, for example under conditions known to those skilled in the art, e.g., in polar solvents like ethanol or DMF, followed by
   when FG is N(H)—PG, removal of the PG protecting group or
   when FG is NO₂, reduction of NO₂ to NH₂.
(g) For compounds of formula V in which R¹ represents —[C(R^{a})(R^{b})]₀₋₂—S—R^{Y1}, oxidation of a compound of formula XXIV,

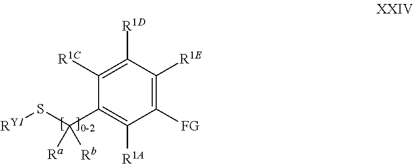

wherein R^{1A}, R^{1C}, R^{1D}, R^{1E}, R^{a}, R^{b}, R^{Y1} and FG are as hereinbefore defined, for example under conditions known to those skilled in the art (e.g. in the presence of a peracid, such as meta-chloroperbenzoic acid) gives the desired sulfoxide or sulfone intermediate. Another route to the sulfone intermediates involves alkylation of the appropriate sodium alkanesulfinate with a compound of the formula XVI. In all cases, the formation of the sulfoxide or sulfone intermediate is followed by
   when FG is N(H)—PG, removal of the PG protecting group or
   when FG is NO₂, reduction of NO₂ to NH₂.
(h) For compounds of formula V in which R¹ represents -L¹-C(O)NR^{X}R^{Y}, -L²-S(O)₂R^{Y1}, -L³-P(O)R^{Y1}R^{Y2}, -L⁴-S(O)₂NR^{X}R^{Y}, -L⁵-C(O)R^{Y} or -L⁷-C(O)OR^{Y}, in which L¹, L², L³, L⁴, L⁵ and L⁷ represent —N(R^{X2})— or —C(R^{a})(R^{b})—N(R^{X2})—, reaction of a compound of formula XXV,

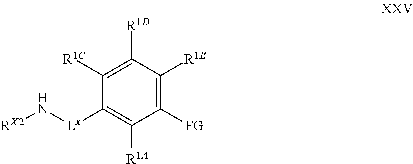

wherein R^{1A}, R^{1C}, R^{1D}, R^{1E}, L^{x}, R^{X2} and FG are as hereinbefore defined, with a compound of formula VIIa, VIIb, VIIc, VIId, VIIe or VIIf, as hereinbefore defined, for example under conditions known to those skilled in the art—e.g., where XXV is condensed with an acid chloride or sulfonyl chloride by condensation in a aprotic solvent, such as dichloromethane or tetrahydrofuran, in the presence of a base, such as diisopropylethylamine—followed by
   when FG is N(H)—PG, removal of the PG protecting group or
   when FG is NO₂, reduction of NO₂ to NH₂.
(i) For compounds of formula V in which R¹ represents —OC(R^{a})(R^{b})—P(O)R^{Y1}R^{Y2}, reaction of a compound of formula XXVI,

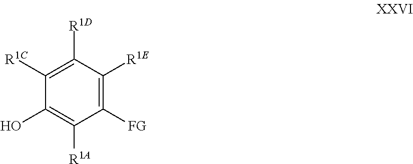

wherein R^{1A}, R^{1C}, R^{1E}, R^{1D} and FG are as hereinbefore defined, with a compound of formula XXVII,

LG⁵-C(R^{a})(R^{b})—P(O)R^{Y1}R^{Y2} XXVII
wherein R^{a}, R^{b}, R^{Y1}, R^{Y2} and LG⁵ are as hereinbefore defined, for example under conditions known to those skilled in the art (e.g. at elevated temperature in the presence of a base, such as K₂CO₃, and a polar, aprotic solvent, such as DMF), followed by
   when FG is N(H)—PG, removal of the PG protecting group or
   when FG is NO₂, reduction of NO₂ to NH₂.
(j) For compounds of formula V in which R¹ represents —O—S(O)₂R^{Y1}, reaction of compound of formula XXVI, as hereinbefore defined, with a sulfonyl chloride of formula XXVIII,

R^{Y1}—SO₂Cl XXVIII
wherein R^{Y1} is as hereinbefore defined, for example, in the presence of a base, such as triethylamine, in an aprotic solvent, such as dichloromethane, followed by
   when FG is N(H)—PG, removal of the PG protecting group or
   when FG is NO₂, reduction of NO₂ to NH₂.

Nitriles of formula XVIII may be prepared by cyanide displacement of LG⁵ in the compound of formula XVI (e.g. with sodium or potassium cyanide in DMSO at ambient temperature). In a similar vein, the amine of formula XXV in which R^{X2} represents H may be prepared, for example, from the corresponding compounds of formula XVI by reaction with an ammonia surrogate, involving, for example, azide displacement followed by Staudinger reduction with triphenylphosphine, or a classical Gabriel amine synthesis comprising reaction with potassium phthalimide followed by cleavage of the imide formed with aqueous or ethanolic hydrazine at reflux.

Compounds of formula XXV in which R^{X2} represents H may also be prepared by reduction of the benzamide XXIX,

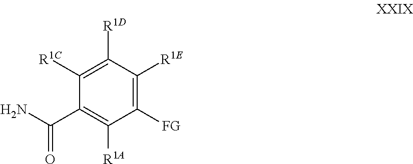

wherein R^{1A}, R^{1C}, R^{1D}, R^{1E} and FG are as hereinbefore defined, for example employing conditions known to those skilled in the art (e.g. reduction with borane or lithium aluminium hydride).

Compounds of formula XVI may themselves be prepared by routes known to those skilled in the art, typically from ketone, carboxylic acid or ester precursors. For example, for compounds of formula XVI in which LG⁵ represents halo may be obtained from the corresponding compounds of formula XIX in which LG² represents OH or O—C₁₋₄ alkyl and —[C(R^{a})(R^{b})]₀₋₂— represents a direct bond or —C(R^{a})(R^{b})—. Reduction of the compound of formula XIX (e.g. when LG² represents OH, with borane or, when LG² represents O—C₁₋₄ alkyl, with lithium aluminium hydride or lithium borohydride in an ethereal solvent) furnishes a benzyl alcohol that can be transformed into the compound of formula XVI by a halogenation reaction employing, for example, thionyl chloride when LG⁵ is chloro or triphenylphosphine and bromine when LG⁵ is bromo.

Compounds of formula XX in which LG² represents OH may be prepared by hydrolysis of nitriles of the formula XIX with aqueous acid or alkali, or with sodium peroxide and water (J. Chem. Soc., Perkin Trans. 2 2000, 2399).

It will be understood by those skilled in the art that the use of appropriate protective groups may be required during the processes using reagents with chemically-sensitive functional groups, for example, hydroxyl or amino groups.

Many of the compounds illustrated in the Schemes are either commercially available, or can be obtained using the cited procedures, or can be readily prepared by conventional methods by those skilled in the art. See for example WO 01/36403, WO 02/083628, WO 2006/039718, WO 2010/026096, WO 2014/027209, WO 2014/076484 and WO 2014/140582.

The aspects of the invention described herein (e.g. the above-mentioned compounds, combinations, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, have a better pharmacokinetic and/or pharmacodynamic profile than, have more suitable solid state morphology than, have better long term stability than, or may have other useful pharmacological properties over, similar compounds, combinations, methods (treatments) or uses known in the prior art for use in the treatment of those conditions or otherwise.

The compounds of the invention may additionally (or alternatively):
(potently) inhibit multiple kinases (e.g. p38 MAP kinase, Syk and Src family kinases);
exhibit a long duration of action and/or persistence of action (e.g. in comparison to other previously disclosed p38 MAP kinase inhibitors such as, for example, BIRB796); not strongly inhibit GSK 3α (e.g. they may have an IC₅₀ against GSK 3α of 1,000 nM or greater; such as 1,500, 2,000, 3,000, 4,000, 5,000, 6,000, 7,000, 8,000, 9,000 or 10,000 nM or greater);
maintain a relatively high drug concentration between doses (e.g. a high concentration relative to other previously disclosed p38 MAP kinase inhibitors such as, for example, BIRB796);
exhibit properties that are particularly suited to topical/local administration (e.g. following topical/local administration, the generation of high target tissue concentrations but low plasma concentrations of the compounds of formula (I) and/or rapid clearance of the compounds of formula (I) from plasma);
exhibit minimal β-catenin induction and/or inhibition of mitosis in cells;
exhibit minimal time-dependent inhibition of members of the cytochrome P450 superfamily
show improved chemical stability in the presence of water (e.g. stability to hydrolysis in aqueous mixtures at elevated temperatures) compared to previously disclosed p38 MAP kinase inhibitors such as, for example, BIRB796;
following administration to a patient, produce metabolites associated with little or no safety (e.g. toxicity) concerns;
exhibit good solubility and/or cellular permeability;
have a high degree of crystallinity; and/or
exhibit little or no hygroscopicity in the solid state.

### Experimental Methods

### General Procedures

All starting materials and solvents were obtained either from commercial sources or prepared according to the literature citation. Unless otherwise stated all reactions were stirred. Organic solutions were routinely dried over anhydrous magnesium sulfate. Hydrogenations were performed on a Thales H-cube flow reactor under the conditions stated or under a balloon of hydrogen. Microwave reactions were performed in a CEM Discover and Smithcreator microwave reactor, heating to a constant temperature using variable power microwave irradiation.

Normal phase column chromatography was routinely carried out on an automated flash chromatography system such as CombiFlash Companion or CombiFlash RF system using pre-packed silica (230-400 mesh, 40-63 μm) cartridges. SCX was purchased from Supelco and treated with 1M hydrochloric acid prior to use. Unless stated otherwise the reaction mixture to be purified was first diluted with MeOH and made acidic with a few drops of AcOH. This solution was loaded directly onto the SCX and washed with MeOH. The desired material was then eluted by washing with 1% NH₃ in MeOH.

### Analytical Methods

Analytical HPLC was carried out using a Waters Xselect CSH C18, 2.5 μm, 4.6×30 mm column eluting with a gradient of 0.1% Formic Acid in MeCN in 0.1% aqueous Formic Acid; a Waters Xbridge BEH C18, 2.5 μm, 4.6×30 mm column eluting with a gradient of MeCN in aqueous 10 mM Ammonium Bicarbonate. UV spectra of the eluted peaks were measured using either a diode array or variable wavelength detector on an Agilent 1100 system.

Analytical LCMS was carried out using a Waters Xselect CSH C18, 2.5 μm, 4.6×30 mm column eluting with a gradient of 0.1% Formic Acid in MeCN in 0.1% aqueous Formic Acid; a Waters Xbridge BEH C18, 2.5 μm, 4.6×30 mm column eluting with a gradient of MeCN in aqueous 10 mM Ammonium Bicarbonate. UV and mass spectra of the eluted peaks were measured using a variable wavelength detector on either an Agilent 1200 with or an Agilent Infinity 1260 LCMS with 6120 single quadrupole mass spectrometer with positive and negative ion electrospray.

Preparative HPLC was carried out using a Waters Xselect CSH C18, 5 μm, 19×50 mm column using either a gradient of either 0.1% Formic Acid in MeCN in 0.1% aqueous Formic Acid or a gradient of MeCN in aqueous 10 mM Ammonium Bicarbonate; or a Waters Xbridge BEH C18, 5 μm, 19×50 mm column using a gradient MeCN in aqueous 10 mM Ammonium Bicarbonate. Fractions were collected following detection by UV at a single wavelength measured by a variable wavelength detector on a Gilson 215 preparative HPLC or Varian PrepStar preparative HPLC; by mass and UV at a single wavelength measured by a ZQ single quadrupole mass spectrometer, with positive and negative ion electrospray, and a dual wavelength detector on a Waters FractionLynx LCMS.

### ¹H NMR Spectroscopy.

¹H NMR spectra were acquired on a Bruker Avance III spectrometer at 400 MHz. Either the central peaks of chloroform-d, dimethylsulfoxide-d₆ or an internal standard of tetramethylsilane were used as references.

### PREPARATION OF COMPOUNDS OF THE INVENTION

### Example 1

### 3-(2-((3-Methoxy-5-(2-(2-(2-methoxyethoxy)ethoxy)ethoxy)phenyl)amino)quinazolin-6-yl)-4-methyl-N-(3-(trifluoromethyl)phenyl)benzamide

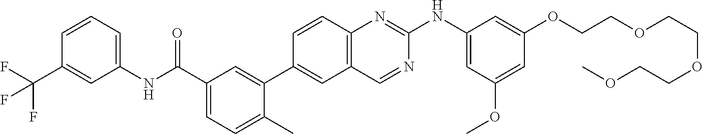 [SMILES: COCCOCCOCCOc1cc(Nc2ncc3cc(-c4cc(C(=O)Nc5cccc(C(F)(F)F)c5)ccc4C)ccc3n2)cc(OC)c1]

### (i) Methyl 3-(2-chloroquinazolin-6-yl)-4-methylbenzoate

A mixture of 6-bromo-2-chloroquinazoline (780 mg, 3.20 mmol) and methyl 4-methyl-3-(4,4,5,5-tetramethyl-1,3,2-dioxaborolan-2-yl)benzoate (1000 mg, 3.62 mmol) in 1,2-dimethoxyethane (3 mL) and 1 M NaHCO₃ solution (10 mL, 10.00 mmol) was purged with nitrogen for 5 minutes. The mixture was heated to 90° C. before adding Pd(Ph₃P)₄ (200 mg, 0.173 mmol) and stirring for 1 h. The mixture was diluted with water (150 mL) and extracted with diethyl ether (3×150 mL). The combined organic phases were washed with water (150 mL), 20% brine (150 mL) and saturated brine (150 mL). The organic phase was dried (MgSO₄) and concentrated to give a yellow oil which was purified by chromatography on the Companion (80 g column, 0-30% EtOAc/isohexane) to afford the sub-title compound (510 mg) as a cream solid.

LCMS m/z 313 (M+H)⁺ (ES⁺)

### (ii) 3-Methoxy-5-nitrophenol

A mixture of KOH (29.0 g, 517 mmol) and 1-bromo-3-methoxy-5-nitrobenzene (30 g, 129 mmol) in water (70 mL) and dioxane (70 mL) was degassed for 5 minutes prior to the addition of di-tert-butyl(2′,4′,6′-triisopropyl-[1,1′-biphenyl]-2-yl)phosphine (1.263 g, 2.97 mmol) and Pd₂(dba)₃ (1.184 g, 1.293 mmol). The resulting mixture was degassed for a further 2 minutes then heated under a nitrogen atmosphere at 100° C. for 2 h. The mixture was cooled, then acidified with 5M HCl to ˜pH 1 and extracted with EtOAc (2×500 mL). The organic layer was washed with saturated brine (200 mL), dried (MgSO₄), filtered and concentrated under reduced pressure. The crude product was purified through a pad of silica eluting with 30% EtOAc/isohexane to afford the sub-title compound (20.76 g) as a yellow solid.

¹H NMR (400 MHz; DMSO-d₆) δ 10.46 (s, 1H), 7.20 (s, 1H), 7.19 (s, 1H), 6.76 (s, 1H), 3.82 (s, 3H).

LCMS m/z 168 (M−H)⁻ (ES⁻)

### (iii) 1-Methoxy-3-(2-(2-(2-methoxyethoxy)ethoxy)ethoxy)-5-nitrobenzene

To a stirred suspension of the product from step (ii) above (8.14 g, 45.7 mmol) and K₂CO₃ (12.64 g, 91 mmol) in acetone (150 mL) was added 1-bromo-2-(2-(2-methoxy-ethoxy)ethoxy)ethane (8.85 mL, 48.0 mmol). The resulting mixture was refluxed overnight, cooled and filtered. The filtrate was evaporated under reduced pressure and the residue purified by chromatography on silica gel (220 g column, 0-60% EtOAc/isohexane) to afford the sub-title compound (13.41 g) as a yellow oil.

¹H NMR (400 MHz, DMSO-d₆) δ: 7.34-7.32 (m, 2H), 6.98 (t, 1H), 4.22-4.20 (m, 2H), 3.85 (s, 3H), 3.77-3.74 (m, 2H), 3.60-3.57 (m, 2H), 3.54-3.50 (m, 4H), 3.44-3.40 (m, 2H), 3.23 (s, 3H).

LCMS m/z 316 (M+H)⁺ (ES⁺)

### (iv) 3-Methoxy-5-(2-(2-(2-methoxyethoxy)ethoxy)ethoxy)aniline

The product from step (iii) above (13.4 g, 42.5 mmol) was dissolved in ethanol (150 mL) and Fe powder (13 g, 233 mmol) was added followed by a solution of NH₄Cl (2.3 g, 43.0 mmol) in water (150 mL). The resulting suspension was heated at 80° C. for 3 h. The reaction was cooled to rt and filtered through Celite. The filtrate was concentrated in vacuo then partitioned between water (250 mL) and EtOAc (400 mL). The organic layer was separated, dried (MgSO₄), filtered and concentrated under reduced pressure. The crude product was purified by chromatography on silica gel (120 g column, 0-4% MeOH/DCM) to afford the sub-title compound (10.95 g) as an oil.

¹H NMR (400 MHz, DMSO-d₆) δ 5.76-5.73 (m, 2H), 5.68 (t, 1H), 5.07 (s, 2H), 3.98-3.89 (m, 2H), 3.72-3.65 (m, 2H), 3.63 (s, 3H), 3.60-3.48 (m, 6H), 3.47-3.40 (m, 2H), 3.24 (s, 3H)

LCMS m/z 286 (M+H)⁺ (ES⁺)

### (v) Methyl 3-(2-((3-methoxy-5-(2-(2-(2-methoxyethoxy)ethoxy)ethoxy)phenyl)amino)-quinazolin-6-yl)-4-methylbenzoate

The product from step (i) above (375 mg, 1.199 mmol), the product from step (iv) above (411 mg, 1.440 mmol) and Cs₂CO₃ (600 mg, 1.842 mmol) were stirred in 1,4-dioxane (9 mL) whilst degassing with nitrogen. A solution of Pd₂(dba)₃ (60 mg, 0.066 mmol) and BINAP (80 mg, 0.128 mmol) in degassed 1,4-dioxane (1 mL) was added and the mixture was heated to 80° C. for 20 h. The mixture was diluted with water (100 mL) and extracted with ethyl acetate (3×50 mL). The combined organic phases were washed with water (50 mL), saturated brine (50 mL), dried (MgSO₄) and concentrated under reduced pressure to yield an orange oil. The crude product was purified by chromatography on the Companion (12 g column, 25-100% EtOAc/isohexane) to afford the sub-title compound (525 mg) as a orange oil.

LCMS m/z 562 (M+H)⁺ (ES⁺)

### (vi) 3-(2-((3-Methoxy-5-(2-(2-(2-methoxyethoxy)ethoxy)ethoxy)phenyl)amino)quinazolin-6-yl)-4-methylbenzoic acid

A solution of the product from step (v) above (560 mg, 0.997 mmol) in THF (6 mL) and methanol (2 mL) was stirred with 2 M NaOH soln (1.6 mL, 3.20 mmol) and water (10 mL) at rt over a weekend. The mixture was concentrated under reduced pressure to remove the organic solvents and then diluted with water (50 mL). The aqueous solution was washed with diethyl ether (3×50 mL) then acidified with 1 M HCl (3.2 mL) and extracted with ethyl acetate (3×50 mL). The combined ethyl acetate solutions were washed with saturated brine (50 mL), dried (MgSO₄) and concentrated under reduced pressure to afford the sub-title compound (430 mg) as an orange waxy solid.

LCMS m/z 548 (M+H)⁺ (ES⁺)

### (vii) 3-(2-((3-Methoxy-5-(2-(2-(2-methoxyethoxy)ethoxy)ethoxy)phenyl)amino)quinazolin-6-yl)-4-methyl-N-(3-(trifluoromethyl)phenyl)benzamide

Et₃N (26.7 μL, 0.192 mmol) was added to a stirred solution of 3-(trifluoromethyl)aniline (22 mg, 0.137 mmol), the product from step (vi) above (70 mg, 0.128 mmol) and HATU (58.3 mg, 0.153 mmol) in N,N-dimethylformamide (1 mL) and the mixture was stirred at rt for 30 minutes. Water (10 mL) was added and the mixture was extracted with ethyl acetate (3×10 mL). The combined organic phases were washed with 20% brine (2×10 mL), saturated brine (10 mL), dried (MgSO₄) and concentrated under reduced pressure. The crude product was purified by chromatography on the Companion (12 g column, EtOAc) to afford the title compound (54 mg) as a yellow glass.

¹H NMR (DMSO-d6) 400 MHz, δ: 10.53 (s, 1H), 9.91 (s, 1H), 9.38 (s, 1H), 8.27-8.22 (m, 1H), 8.12-8.06 (m, 1H), 8.03-7.99 (m, 2H), 7.96 (dd, 1H), 7.92 (dd, 1H), 7.78 (d, 1H), 7.64-7.57 (m, 1H), 7.55 (d, 1H), 7.49-7.43 (m, 1H), 7.37 (dd, 1H), 7.32 (dd, 1H), 6.20 (dd, 1H), 4.14-4.06 (m, 2H), 3.82-3.73 (m, 2H), 3.78 (s, 3H), 3.65-3.59 (m, 2H), 3.59-3.51 (m, 4H), 3.47-3.42 (m, 2H), 3.24 (s, 3H), 2.39 (s, 3H).

LCMS m/z 691 (M+H)⁺ (ES⁺); 689 (M−H)⁻ (ES⁻)

### Example 2

### N-(5-(tert-Butyl)-2-methoxy-3-(methylsulfonamido)phenyl)-3-(2-((3-methoxy-5-(2-(2-(2-methoxyethoxy)ethoxy)ethoxy)phenyl)amino)quinazolin-6-yl)-4-methylbenzamide

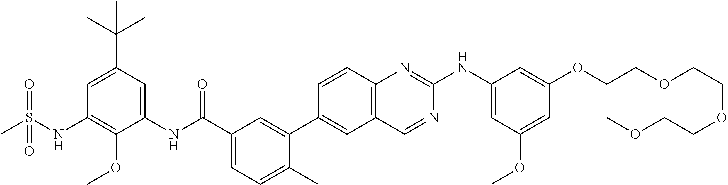 [SMILES: COCCOCCOCCOc1cc(Nc2ncc3cc(-c4cc(C(=O)Nc5cc(C(C)(C)C)cc(NS(C)(=O)=O)c5OC)ccc4C)ccc3n2)cc(OC)c1]

Et₃N (26.7 μL, 0.192 mmol) was added to a stirred solution of N-(3-amino-5-(tert-butyl)-2-methoxyphenyl)methanesulfonamide (see, for example, Cirillo, P. F. et al., WO 2002/083628, 24 Oct. 2002; 35 mg, 0.129 mmol), 3-(2-((3-methoxy-5-(2-(2-(2-methoxyethoxy)ethoxy)-ethoxy)phenyl)amino)quinazolin-6-yl)-4-methylbenzoic acid (see Example 1(vi) above; 70 mg, 0.128 mmol) and HATU (58.3 mg, 0.153 mmol) in N,N-dimethylformamide (1 mL) and the mixture was stirred at rt for 3 h. Slow conversion was observed so the mixture was heated to 45° C. and stirred over 18 h. Water (10 mL) was added and the mixture was extracted with ethyl acetate (3×10 mL). The combined organic phases were washed with 20% brine (2×10 mL), saturated brine (10 mL), dried (MgSO₄) and concentrated under reduced pressure. The crude product was purified by chromatography on the Companion (12 g column, EtOAc) to afford the title compound (56 mg) as a yellow solid.

¹H NMR (DMSO-d6) 400 MHz, δ: 9.90 (s, 1H), 9.89 (s, 1H), 9.38 (s, 1H), 9.14 (s, 1H), 8.02-7.97 (m, 2H), 7.97-7.90 (m, 2H), 7.78 (d, 1H), 7.52 (d, 1H), 7.46 (d, 1H), 7.37 (dd, 1H), 7.32 (dd, 1H), 7.25 (d, 1H), 6.20 (dd, 1H), 4.14-4.07 (m, 2H), 3.81-3.75 (m, 2H), 3.78 (s, 3H), 3.71 (s, 3H), 3.65-3.59 (m, 2H), 3.59-3.51 (m, 4H), 3.47-3.42 (m, 2H), 3.24 (s, 3H), 3.05 (s, 3H), 2.39 (s, 3H), 1.28 (s, 9H).

LCMS m/z 802 (M+H)⁺ (ES⁺); 800 (M−H)⁻ (ES⁻)

### Example 3

### N-(5-(tert-Butyl)-3-carbamoyl-2-methoxyphenyl)-3-(2-((3-methoxy-5-((2-morpholinoethyl)-carbamoyl)phenyl)amino)quinazolin-6-yl)-4-methylbenzamide

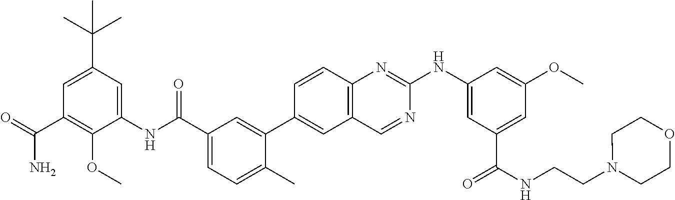 [SMILES: COc1cc(Nc2ncc3cc(-c4cc(C(=O)Nc5cc(C(C)(C)C)cc(C(N)=O)c5OC)ccc4C)ccc3n2)cc(C(=O)NCCN2CCOCC2)c1]

### (i) 3-Amino-5-methoxy-N-(2-morpholinoethyl)benzamide

To a stirred mixture of 3-amino-5-methoxybenzoic acid (5.20 g, 31.1 mmol), Et₃N (4.50 mL, 32.3 mmol) and 2-morpholinoethanamine (4.23 mL, 32.3 mmol) in THF (150 mL) and DMF (4 mL) was added HATU (14.72 g, 38.7 mmol) and the reaction stirred at ambient temperature overnight. After this time the mixture was taken up in ethyl acetate (300 mL) and washed with sat NaHCO₃ (aq) (2×100 mL). The aqueous was back extracted with further ethyl acetate (4×50 mL) and organics combined, dried over MgSO₄, filtered and concentrated under reduced pressure. Trituration with isohexanes (100 mL) afforded a pale orange gum (15 g). The crude product was purified by chromatography on the Companion (220 g column, 0-60% IPA in DCM). Fractions were combined as two separate batches to afford the sub-title compound as two separate batches (2.48 g and 2.87 g) as orange solids.

¹H NMR (400 MHz; CDCl₃) δ: 6.69-6.64 (m, 3H), 6.35 (t, 1H), 3.81 (br.s, 2H), 3.81 (s, 3H), 3.73 (m, 4H), 3.53 (dd, 2H), 2.62-2.57 (m, 2H), 2.53-2.49 (m, 4H).

LCMS m/z 280 (M+H)⁺ (ES⁺)

The first batch (2.0 g) was recrystallised in acetonitrile (18 mL) to yield the sub-title compound (1.70 g) as a white solid which was used in the next step.

### (ii) Methyl 3-(2-((3-methoxy-5-((2-morpholinoethyl)carbamoyl)phenyl)amino)quinazolin-6-yl)-4-methylbenzoate

A solution of methyl 3-(2-chloroquinazolin-6-yl)-4-methylbenzoate (see Example 1(i) above; 1.45 g, 4.64 mmol), the product from step (i) above (1.360 g, 4.87 mmol) and p-TSA monohydrate (1.323 g, 6.95 mmol) in dry DMF (50 mL) was stirred at 85° C. for 18 h. The reaction mixture was cooled to rt and partitioned between EtOAc (200 mL) and sat. NaHCO₃ solution (300 mL). The organics were washed with brine (5×200 mL), dried (MgSO₄) and evaporated to dryness. The crude product was purified by chromatography on silica gel (120 g column, 0-10% MeOH/DCM) to afford the sub-title compound (2.0 g) as a yellow solid.

¹H NMR (400 MHz, DMSO-d6) δ 10.11 (s, 1H), 9.40 (d, 1H), 8.34 (t, 1H), 8.02 (d, 1H), 7.99 (d, 1H), 7.95-7.85 (m, 4H), 7.76 (d, 1H), 7.54 (d, 1H), 7.02 (dd, 1H), 3.87 (s, 3H), 3.86 (s, 3H), 3.59 (t, 4H), 3.40 (q, 2H), 2.44 (m, 6H), 2.38 (s, 3H).

LCMS m/z 556 (M+H)⁺ (ES⁺); 554 (M−H)⁻ (ES⁻)

### (iii) 3-(2-((3-Methoxy-5-((2-morpholinoethyl)carbamoyl)phenyl)amino)quinazolin-6-yl)-4-methylbenzoic acid

A suspension of the product from step (ii) above (2.0 g, 3.60 mmol) in THF (50 mL) and MeOH (25 mL) was treated with LiOH (0.431 g, 18.00 mmol) followed by water (10 mL) and stirred at rt for 18 h. The reaction mixture was acidified with 1 M HCl (to pH 3) and extracted with EtOAc (3×50 mL). LCMS showed desired product in aqueous phase with small amount in organics. Both phases were passed through SAX resin (loading MeOH) and then eluted with 5% AcOH/MeOH. The washings were evaporated to afford a yellow solid that was treated with MeOH (100 mL) and the white solid removed by filtration. The filtrate was evaporated and the residue adsorbed on to silica and purified by chromatography on silica gel (80 g column, 0-20% MeOH/DCM with 1% NH₄OH). The product was dried overnight under vacuum at 40° C. to afford the sub-title compound (918 mg) as a yellow solid.

¹H NMR (400 MHz, DMSO-d6) δ 10.09 (s, 1H), 9.39 (d, 1H), 8.34 (t, 1H), 8.03 (t, 1H), 7.96 (d, 1H), 7.92 (t, 1H), 7.89-7.78 (m, 3H), 7.75 (d, 1H), 7.40 (d, 1H), 7.01 (dd, 1H), 3.86 (s, 3H), 3.59 (t, 4H), 3.45-3.36 (m, 2H), 2.45 (d, 6H), 2.34 (s, 3H).

LCMS m/z 542 (M+H)⁺ (ES⁺); 540 (M−H)⁻ (ES⁻)

### (iv) N-(5-(tert-Butyl)-3-carbamoyl-2-methoxyphenyl)-3-(2-((3-methoxy-5-((2-morpholinoethyl)carbamoyl)phenyl)amino)quinazolin-6-yl)-4-methylbenzamide

A suspension of the product from step (iii) above (200 mg, 0.369 mmol) and 3-amino-5-(tert-butyl)-2-methoxybenzamide (90 mg, 0.406 mmol) in dry DMF (3 mL) was treated with DIPEA (193 μL, 1.108 mmol) then HATU (154 mg, 0.406 mmol) and the reaction mixture stirred at rt for 72 h. HATU (154 mg, 0.406 mmol) was added and the reaction mixture stirred for 1 h. Reaction mixture did not change so the solution was loaded directly on to SCX resin (load MeOH; eluted 1% NH₃/MeOH) and the eluted product evaporated. The crude product was purified by chromatography on silica gel (12 g column, 0-10% MeOH/DCM) to afford the title compound (30 mg) as a yellow solid.

¹H NMR (400 MHz, DMSO-d6) δ 10.10 (s, 1H), 9.89 (s, 1H), 9.41 (d, 1H), 8.33 (t, 1H), 8.05-7.91 (m, 5H), 7.80-7.75 (m, 2H), 7.72 (s, 1H), 7.57 (s, 1H), 7.53 (d, 1H), 7.50 (d, 1H), 7.02 (dd, 1H), 3.86 (s, 3H), 3.74 (s, 3H), 3.59 (t, 4H), 3.40 (q, 2H), 2.46 (d, 6H), 2.40 (s, 3H), 1.30 (s, 9H).

LCMS m/z 746 (M+H)⁺ (ES⁺); 744 (M−H)⁻ (ES⁻)

### Example 4

### N-(5-(tert-Butyl)-3-(dimethylphosphoryl)-2-methoxyphenyl)-3-(2-((3-methoxy-5-((2-morpholinoethyl)carbamoyl)phenyl)amino)quinazolin-6-yl)-4-methylbenzamide

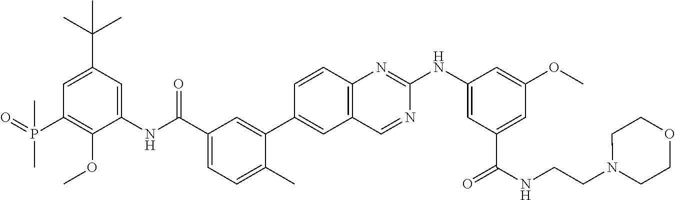 [SMILES: COc1cc(Nc2ncc3cc(-c4cc(C(=O)Nc5cc(C(C)(C)C)cc(P(C)(C)=O)c5OC)ccc4C)ccc3n2)cc(C(=O)NCCN2CCOCC2)c1]

### (i) 5-(tert-Butyl)-1-iodo-2-methoxy-3-nitrobenzene

To a stirred solution of 5-(tert-butyl)-2-methoxy-3-nitroaniline (3.7 g, 16.50 mmol) and iodine (2.5 g, 9.85 mmol) in toluene (50 mL) at 0° C. was added tert-butyl nitrite (2.5 mL, 18.92 mmol) and the reaction warmed to rt and stirred overnight. The reaction was diluted with EtOAc (100 ml) and washed with brine (60 mL) then sat. aq. sodium thiosulfate solution (60 mL). The organic phase was dried (MgSO₄), filtered and concentrated in vacuo affording a red oil. The crude product was purified by chromatography on the Companion (120 g column, 0-5% EtOAc in hexane) to afford the sub-title compound (4 g) as a yellow oil.

¹H NMR (400 MHz, DMSO-d6) δ 8.11 (d, 1H), 7.89 (d, 1H), 3.85 (s, 3H), 1.29 (s, 9H).

### (ii) 5-(tert-Butyl)-3-iodo-2-methoxyaniline

Fe powder (7.00 g, 125 mmol) was added to a suspension of the product from step (i) above (4.2 g, 12.53 mmol) in ethanol (30 mL) and NH₄Cl (1 g, 18.69 mmol) in water (15 mL). The solution was stirred vigorously (overhead stirrer) at 95° C. (block temperature, internal temperature 75° C.) for 1 h. The reaction mixture was filtered through Celite and the filtrate diluted with water (100 mL). The product was extracted with EtOAc (3×100 mL). The organics were bulked, dried (MgSO₄), filtered and evaporated to a dark brown oil. The crude product was purified by chromatography on silica gel (80 g column, 10% EtOAc:isohexane to 20%) to afford the sub-title compound (3.5 g) as cream crystalline solid.

¹H NMR (400 MHz, DMSO-d6) δ 6.88 (d, 1H), 6.73 (d, 1H), 5.07 (s, 2H), 3.60 (s, 3H), 1.19 (s, 9H).

LCMS m/z 306 (M+H)⁺ (ES⁺)

### (iii) (3-Amino-5-(tert-butyl)-2-methoxyphenyl)dimethylphosphine oxide

Dimethylphosphine oxide (1 mL, 15.89 mmol) was added to a degassed suspension of the product from step (ii) above (3.5 g, 11.47 mmol), Pd(OAc)₂ (250 mg, 1.114 mmol), xantphos (1.3 g, 2.247 mmol) and finely powdered potassium phosphate tribasic (6 g, 28.3 mmol) in DMF (50 mL) and stirred vigorously under nitrogen at 150° C. block temperature for 1 h. The mixture was diluted with DCM (100 mL) and filtered through Celite. The filtrate was evaporated to a dark gum. The crude product was purified by chromatography on silica gel (80 g column, 2% MeOH:EtOAc to 10%) to afford the sub-title compound (2 g) as a yellow solid.

¹H NMR (400 MHz, DMSO-d6) δ 6.97 (d, 1H), 6.89 (dd, 1H), 5.00 (s, 2H), 3.72 (s, 3H), 1.64 (d, 6H), 1.24 (s, 9H).

LCMS m/z 256 (M+H)⁺ (ES⁺)

### (iv) N-(5-(tert-Butyl)-3-(dimethylphosphoryl)-2-methoxyphenyl)-3-(2-((3-methoxy-5-((2-morpholinoethyl)carbamoyl)phenyl)amino)quinazolin-6-yl)-4-methylbenzamide

A suspension of 3-(2-((3-methoxy-5-((2-morpholinoethyl)carbamoyl)phenyl)amino)quinazolin-6-yl)-4-methylbenzoic acid (see Example 3(iii) above; 150 mg, 0.277 mmol) and the product from step (iii) above (78 mg, 0.305 mmol) in dry DMF (2 mL) was treated with DIPEA (145 μL, 0.831 mmol) then HATU (116 mg, 0.305 mmol) and the reaction mixture stirred at rt for 72 h. Reaction mixture was partitioned between EtOAc (20 mL) and sat. NaHCO₃ solution (20 mL). The organics were washed with brine (5×20 mL) and dried (MgSO₄). The crude product was purified by chromatography on silica gel (12 g column, 0-10% MeOH/DCM with 1% NH₃) followed by preparative HPLC (Gilson, Basic (0.1% Ammonium Bicarbonate), Basic, Waters X-Bridge Prep-C18, 5 μm, 19×50 mm column, 5-95% MeCN in Water) then dried at 40° C. under vacuum for 24 h to afford the title compound (34 mg) as a yellow solid.

¹H NMR (400 MHz, DMSO-d6) δ 10.14 (s, 1H), 10.08 (s, 1H), 9.41 (s, 1H), 8.31 (d, 1H), 8.08-7.90 (m, 6H), 7.78 (d, 1H), 7.68 (d, 1H), 7.64 (d, 1H), 7.53 (d, 1H), 7.02 (s, 1H), 3.86 (s, 3H), 3.78 (s, 3H), 3.59 (t, 4H), 3.37 (m, 2H), 2.48-2.41 (m, 6H), 2.40 (s, 3H), 1.68 (d, 6H), 1.31 (s, 9H).

LCMS m/z 779 (M+H)⁺ (ES⁺); 777 (M−H)⁻ (ES⁻)

### Example 5

### N-(5-(tert-butyl)-2-methoxy-3-(methylsulfonamido)phenyl)-3-(2-((3-methoxy-5-((2-morpholinoethyl)carbamoyl)phenyl)amino)quinazolin-6-yl)-4-methylbenzamide

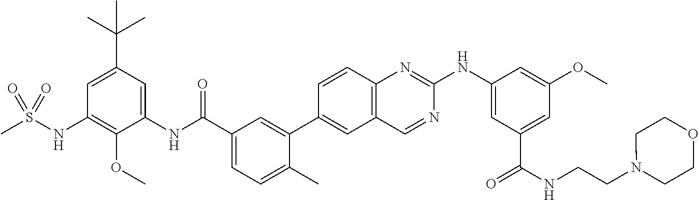 [SMILES: COc1cc(Nc2ncc3cc(-c4cc(C(=O)Nc5cc(C(C)(C)C)cc(NS(C)(=O)=O)c5OC)ccc4C)ccc3n2)cc(C(=O)NCCN2CCOCC2)c1]

A solution of 3-(2-((3-methoxy-5-((2-morpholinoethyl)carbamoyl)phenyl)amino)quinazolin-6-yl)-4-methylbenzoic acid (see Example 3(iii) above; 200 mg, 0.369 mmol), N-(3-amino-5-(tert-butyl)-2-methoxyphenyl)methanesulfonamide (see, for example, Cirillo, P. F. et al., WO 2002/083628, 24 Oct. 2002; 101 mg, 0.369 mmol) and DIPEA (193 μL, 1.108 mmol) in dry DMF (5 mL) was treated with HATU (154 mg, 0.406 mmol) and the reaction mixture stirred at 60° C. for 18 h and then cooled to rt and stirred for 48 h. Reaction mixture was partitioned between EtOAc (40 mL) and sat. NaHCO₃ soln (40 mL). The organics were washed with brine (5×40 mL) and dried (MgSO₄). The crude product was purified by chromatography on silica gel (12 g column, 0-10% MeOH/DCM with 1% NH₃) followed by preparative HPLC (Gilson, Basic (0.1% Ammonium Bicarbonate), Basic, Waters X-Bridge Prep-C18, 5 μm, 19×50 mm column, 5-95% MeCN in water) then dried at 40° C. under vacuum for 24 h to afford the title compound (60 mg) as a yellow solid.

¹H NMR (400 MHz, DMSO-d6) δ 10.08 (s, 1H), 9.89 (s, 1H), 9.44-9.39 (m, 1H), 9.14 (s, 1H), 8.33-8.27 (m, 1H), 8.06-7.88 (m, 6H), 7.80-7.75 (m, 1H), 7.56-7.50 (m, 1H), 7.49-7.44 (m, 1H), 7.27-7.22 (m, 1H), 7.06-6.98 (m, 1H), 3.86 (s, 3H), 3.71 (s, 3H), 3.63-3.54 (m, 4H), 3.45-3.36 (m, 2H), 3.05 (s, 3H), 2.49-2.42 (m, 6H), 2.40 (s, 3H), 1.28 (s, 9H).

LCMS m/z 796 (M+H)⁺ (ES⁺); 794 (M−H)⁻ (ES⁻)

### Example 6

The following compounds are prepared by methods analogous to those described above.

### (a) N-(5-tert-Butyl-2-methoxy-3-methylsulfonyl-phenyl)-3-[2-[3-methoxy-5-(2-morpholinoethyl-carbamoyl)anilino]quinazolin-6-yl]-4-methyl-benzamide

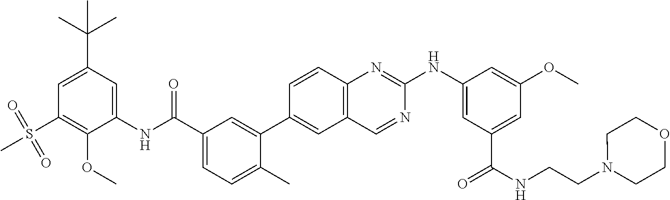 [SMILES: COc1cc(Nc2ncc3cc(-c4cc(C(=O)Nc5cc(C(C)(C)C)cc(S(C)(=O)=O)c5OC)ccc4C)ccc3n2)cc(C(=O)NCCN2CCOCC2)c1]

### (b) N-(5-tert-Butyl-2-methoxy-3-methylsulfinyl-phenyl)-3-[2-[3-methoxy-5-(2-morpholinoethyl-carbamoyl)anilino]quinazolin-6-yl]-4-methyl-benzamide

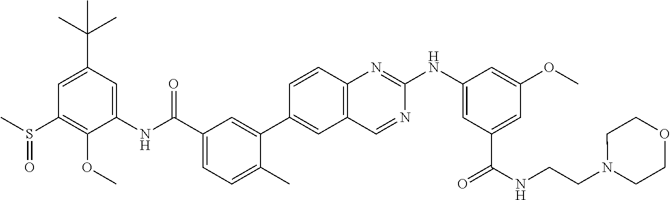 [SMILES: COc1cc(Nc2ncc3cc(-c4cc(C(=O)Nc5cc(C(C)(C)C)cc(S(C)=O)c5OC)ccc4C)ccc3n2)cc(C(=O)NCCN2CCOCC2)c1]

### Example 7

### N-(5-(tert-Butyl)-2-methoxy-3-(methylsulfonamido)phenyl)-3-(2-((4-((dimethyl(oxo)-I6-sulfanylidene)amino)phenyl)amino)quinazolin-6-yl)-4-methylbenzamide

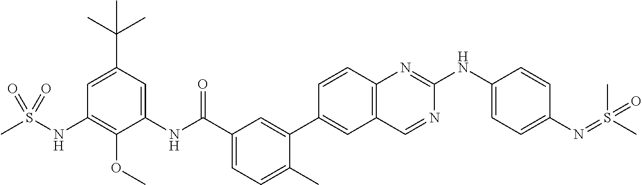

### (i) 6-Bromo-2-(methylthio)quinazoline

A suspension of 6-bromo-2-chloroquinazoline (22.52 g, 92 mmol) in dry DMF (200 mL) was cooled to 0° C. and treated dropwise with a solution/slurry of sodium thiomethoxide (6.45 g, 92 mmol) in dry DMF (100 mL). The reaction mixture was slowly warmed to rt and stirred for 2 h. The reaction mixture was diluted with EtOAc (1 L) and partitioned with water (1 L). The organics were washed with brine (4×500 mL), dried (MgSO₄), and evaporated. The yellow solid was triturated from 50% diethyl ether/iso-hexanes, washing with fresh diethyl ether and drying under suction to afford the sub-title compound (16.59 g) as a yellow solid.

¹H NMR (400 MHz, Chloroform-d) δ 9.10 (d, 1H), 8.01 (dd, 1H), 7.92 (dd, 1H), 7.77 (dt, 1H), 2.70 (s, 3H).

### (ii) Methyl 4-methyl-3-(2-(methylthio)quinazolin-6-yl)benzoate

A suspension of the product from step (i) above (12.93 g, 50.7 mmol), methyl 4-methyl-3-(4,4,5,5-tetramethyl-1,3,2-dioxaborolan-2-yl)benzoate (14.0 g, 50.7 mmol) and cesium carbonate (49.75 g, 153 mmol) in THF/dioxane/water (200 mL; 2:2:1) was degassed with N₂ (under sonication) and then treated with PdCl₂(PPh₃)₂ (3.56 g, 5.07 mmol). The suspension was further degassed and then stirred at 70° C. for 24 h. The reaction mixture was then held at rt for 48 h. The reaction mixture was partitioned between EtOAc (1 L) and 20% brine (1 L) and the organics washed with brine (2×1 L), dried (MgSO₄) and evaporated to afford a brown oil. The crude product was purified by chromatography on silica gel (330 g column, 0-20% EtOAc/iso-hexanes). The resulting yellow solid was triturated from 30% diethyl ether/iso-hexanes to afford the sub-title compound (9.04 g) as an off-white solid. The liquours were evaporated to afford a second crop of the sub-title compound (3.02 g) as a yellow solid.

¹H NMR (400 MHz, DMSO-d6) δ 9.46 (d, 1H), 8.12 (dd, 1H), 8.01 (dd, 1H), 7.97-7.86 (m, 3H), 7.58-7.50 (m, 1H), 3.87 (s, 3H), 2.66 (s, 3H), 2.36 (s, 3H).

### (iii) 4-Methyl-3-(2-(methylthio)quinazolin-6-yl)benzoic acid

A solution of the product from step (ii) above (9.04 g, 27.9 mmol) in THF (150 mL) and MeOH (60 mL) was treated with aq. sodium hydroxide (16.72 ml, 33.4 mmol) and the reaction mixture stirred at rt for 18 h. Additional aq. sodium hydroxide (2.79 ml, 5.57 mmol) was added and the reaction mixture stirred at rt for 72 h. The reaction mixture was acidified with 1M HCl (to pH 1) and the white solid filtered, washed with water, diethyl ether, dried under suction and then dried under vacuum at 40° C. for 24 h to afford the sub-title compound (8.66 g) as a white solid.

¹H NMR (400 MHz, DMSO-d6) δ 12.98 (s, 1H), 9.46 (d, 1H), 8.12 (dd, 1H), 8.01 (dd, 1H), 7.97-7.84 (m, 3H), 7.51 (d, 1H), 2.66 (s, 3H), 2.36 (s, 3H).

### (iv) N-(5-(tert-Butyl)-2-methoxy-3-(methylsulfonamido)phenyl)-4-methyl-3-(2-(methylthio)quinazolin-6-yl)benzamide

A suspension of the product from step (iii) above (7.63 g, 24.58 mmol) and N-(3-amino-5-(tert-butyl)-2-methoxyphenyl)methanesulfonamide (8.01 g, 29.4 mmol) in dry DMF (130 mL) was cooled to 0° C. and treated slowly with DIPEA (12.88 ml, 73.8 mmol). The reaction was stirred for 10 mins until a solution formed. HATU (11.22 g, 29.5 mmol) was added portionwise and the reaction mixture allowed to reach rt and stirred for 18 h. The reaction mixture was treated with water (500 mL) and the slurry stirred for 30 mins. The resulting suspension was filtered and dried under suction. The solid was dissolved in DCM (80 mL) and partitioned with water (80 mL). The organics were separated through a hydrophobic frit, and evaporated and the crude product was purified by chromatography on silica gel (330 g column, 0-30% then to 45% EtOAc/iso-hexanes) to afford the sub-title compound (8.73 g) as a pale yellow solid.

¹H NMR (400 MHz, DMSO-d6) δ 9.90 (s, 1H), 9.47 (d, 1H), 9.15 (s, 1H), 8.16 (d, 1H), 8.07 (dd, 1H), 8.01-7.92 (m, 3H), 7.54 (d, 1H), 7.45 (d, 1H), 7.25 (d, 1H), 3.70 (s, 3H), 3.04 (s, 3H), 2.66 (s, 3H), 2.38 (s, 3H), 1.28 (s, 9H).

LCMS m/z 565 (M+H)⁺ (ES⁺); 563 (M−H)⁻ (ES⁻)

### (v) N-(5-(tert-Butyl)-2-methoxy-3-(methylsulfonamido)phenyl)-4-methyl-3-(2-(methylsulfinyl)quinazolin-6-yl)benzamide

A solution of the product from step (iv) above (11.0 g, 19.48 mmol) in dry DCM (375 mL) was cooled to 0° C. and treated portionwise with mCPBA (3.70 g, 21.43 mmol). The reaction mixture was slowly warmed to rt and stirred for 18 h. The reaction mixture was cooled to 0° C. and mCPBA (1.008 g, 5.84 mmol) was added and the reaction mixture warmed to rt and stirred for 2 h. The reaction mixture was partitioned with water (500 mL) and the organics washed with brine (2×500 mL), dried (MgSO₄) and evaporated. The residue was adsorbed onto silica and the crude product was purified by chromatography on silica gel (330 g column, 0-100% iso-hexanes/EtOAc then 0-8% MeOH/DCM) to afford the sub-title compound (8.89 g) as a pale yellow solid.

¹H NMR (400 MHz, DMSO-d6) δ 9.92 (s, 1H), 9.83 (d, 1H), 9.15 (s, 1H), 8.38 (dd, 1H), 8.31-8.23 (m, 2H), 8.03 (s, 2H), 7.57 (d, 1H), 7.47 (d, 1H), 7.25 (d, 1H), 3.71 (s, 3H), 3.05 (s, 3H), 2.99 (s, 3H), 2.40 (s, 3H), 1.28 (s, 9H).

### (vi) N-(5-(tert-Butyl)-2-methoxy-3-(methylsulfonamido)phenyl)-3-(2-((4-((dimethyl(oxo)-I6-sulfanylidene)amino)phenyl)amino)quinazolin-6-yl)-4-methylbenzamide

A solution of the product from step (v) above (90 mg, 0.155 mmol) in dry DMF (1 mL) was treated with tosic acid (8.0 mg, 0.047 mmol. solution in 0.5 mL dry DMF) followed by ((4-aminophenyl)imino)dimethyl-I6-sulfanone (57 mg, 0.310 mmol) and the reaction mixture warmed to 70° C. and stirred for 18 h. The crude product was purified by preparative HPLC (Waters, Acidic (0.1% Formic acid), Acidic, Waters X-Select Prep-C18, 5 μm, 19×50 mm column, 35-65% MeCN in Water) to afford the title compound (18 mg) as a yellow solid.

¹H NMR (400 MHz, DMSO-d6) δ 9.89 (s, 1H), 9.73 (s, 1H), 9.31 (d, 1H), 9.14 (s, 1H), 8.02-7.91 (m, 3H), 7.88 (dd, 1H), 7.81 (d, 2H), 7.70 (d, 1H), 7.51 (d, 1H), 7.46 (d, 1H), 7.25 (d, 1H), 6.97-6.90 (m, 2H), 3.71 (s, 3H), 3.20 (s, 6H), 3.05 (s, 3H), 2.40 (s, 3H), 1.28 (s, 9H).

LCMS m/z 701 (M+H)⁺ (ES⁺)

### Example 8

### 4-((6-(5-((5-(tert-Butyl)-2-methoxy-3-(methylsulfonamido)phenyl)carbamoyl)-2-methylphenyl)quinazolin-2-yl)amino)-2-methoxybenzoic acid

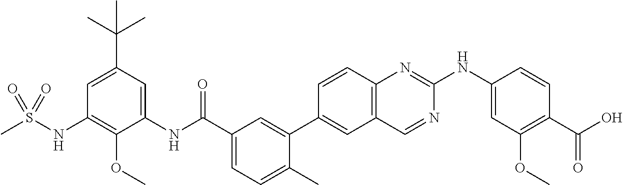 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)O]

### (i) Methyl 4-((6-(5-((5-(tert-butyl)-2-methoxy-3-(methylsulfonamido)phenyl)carbamoyl)-2-methylphenyl)quinazolin-2-yl)amino)-2-methoxybenzoate

A solution of N-(5-(tert-butyl)-2-methoxy-3-(methylsulfonamido)phenyl)-4-methyl-3-(2-(methylsulfinyl)quinazolin-6-yl)benzamide (see Example 7(v) above; 2.02 g, 3.48 mmol), methyl 4-amino-2-methoxybenzoate (1.29 g, 7.12 mmol) and tosic acid (0.198 g, 1.044 mmol) in dry DMF (30 mL) was warmed to 70° C. and stirred for 18 h. The reaction mixture was partitioned between EtOAc (50 mL) and water (50 mL). The organics were washed with brine (5×50 mL), dried (MgSO₄) and evaporated. The crude product was purified by chromatography on silica gel (80 g column, 0-100% EtOAc/iso-hexanes. Product eluted at 70%) to afford the sub-title compound (1.45 g) as a pale yellow solid.

¹H NMR (400 MHz, DMSO-d6) δ 10.35 (s, 1H), 9.88 (s, 1H), 9.46 (d, 1H), 9.14 (s, 1H), 8.22 (d, 1H), 8.05 (d, 1H), 7.98 (dd, 3H), 7.86 (d, 1H), 7.74 (d, 1H), 7.56-7.50 (m, 2H), 7.47 (d, 1H), 7.25 (d, 1H), 3.91 (s, 3H), 3.77 (s, 3H), 3.71 (s, 3H), 3.05 (s, 3H), 2.39 (s, 3H), 1.28 (s, 9H).

LCMS m/z 698 (M+H)⁺ (ES⁺); 696 (M−H)⁻ (ES⁻)

### (ii) 4-((6-(5-((5-(tert-Butyl)-2-methoxy-3-(methylsulfonamido)phenyl)carbamoyl)-2-methylphenyl)quinazolin-2-yl)amino)-2-methoxybenzoic acid

A suspension of the product from step (i) above (1.45 g, 2.078 mmol) in THF/MeOH (1:1; 12 mL) was treated with aq. sodium hydroxide (1.351 mL, 2.70 mmol) and the resultant solution stirred at rt for 18 h. Additional aq. sodium hydroxide (2.60 ml, 5.19 mmol) was added and the reaction mixture stirred at rt for 72 h. The reaction mixture was cooled to 0° C. and acidified to pH1 with 1M HCl. The resulting yellow solid was filtered, washed with water, diethyl ether and dried under suction. The solid was further dried for 18 h under vacuum at 40° C. to afford the title compound (1.24 g) as an orange solid.

¹H NMR (400 MHz, DMSO-d6) δ 10.31 (s, 1H), 9.89 (s, 1H), 9.45 (d, 1H), 9.14 (s, 1H), 8.19 (d, 1H), 8.05 (d, 1H), 8.02-7.93 (m, 3H), 7.85 (d, 1H), 7.75 (d, 1H), 7.55-7.49 (m, 2H), 7.47 (d, 1H), 7.25 (d, 1H), 3.92 (s, 3H), 3.71 (s, 3H), 3.05 (s, 3H), 2.39 (s, 3H), 1.28 (s, 9H).

LCMS m/z 684 (M+H)⁺ (ES⁺); 682 (M−H)⁻ (ES⁻)

### Example 9

### 4-((6-(5-((5-(tert-Butyl)-2-methoxy-3-(methylsulfonamido)phenyl)carbamoyl)-2-methylphenyl)quinazolin-2-yl)amino)-2-methoxy-N-methyl-N-(2-morpholinoethyl)benzamide

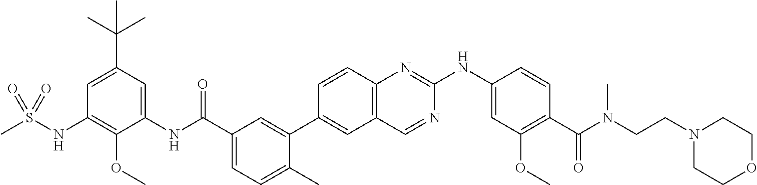 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)N(C)CCN1CCOCC1]

HATU (49 mg, 0.129 mmol) was added to a stirred solution of 4-((6-(5-((5-(tert-butyl)-2-methoxy-3-(methylsulfonamido)phenyl)carbamoyl)-2-methylphenyl)quinazolin-2-yl)amino)-2-methoxybenzoic acid (see Example 8 above; 80 mg, 0.117 mmol), N-methyl-2-morpholinoethanamine (20 mg, 0.139 mmol) and DIPEA (62 μL, 0.355 mmol) in DMF (3 mL) at rt. The mixture was stirred for 3 h. The mixture was poured into water (10 mL). The organic layer was extracted with DCM (10 mL) and dried via a hydrophobic phase separator. The crude product was purified by preparative HPLC (Varian, Basic (0.1% Ammonium Bicarbonate), Basic, Waters X-Bridge Prep-C18, 5 μm, 19×50 mm column, 20-50% MeCN in Water) to afford the title compound (21 mg) as a yellow solid.

¹H NMR (400 MHz, DMSO-d6) δ 10.10 (s, 1H), 9.87 (s, 1H), 9.40 (s, 1H), 9.14 (s, 1H), 8.07-7.92 (m, 5H), 7.80 (d, 1H), 7.54-7.50 (m, 2H), 7.45 (d, 1H), 7.24 (d, 1H), 7.11 (dd, 1H), 3.86 (s, 3H), 3.70 (s, 3H), 3.59 (t, 2H), 3.55 (m, 1H), 3.47 (t, 2H), 3.25 (bs, 1H), 3.04 (s, 3H), 2.97 (s, 1.5H), 2.82 (s, 1.5H), 2.45 (m, 3H), 2.39 (s, 3H), 2.19 (m, 2H), 1.27 (s, 9H). (1 proton under DMSO peak)

LCMS m/z 810 (M+H)⁺ (ES⁺)

### Example 10

### 3-((6-(5-((5-(tert-Butyl)-2-methoxy-3-(methylsulfonamido)phenyl)carbamoyl)-2-methylphenyl)-quinazolin-2-yl)amino)-5-((2-morpholinoethyl)carbamoyl)phenyl methanesulfonate

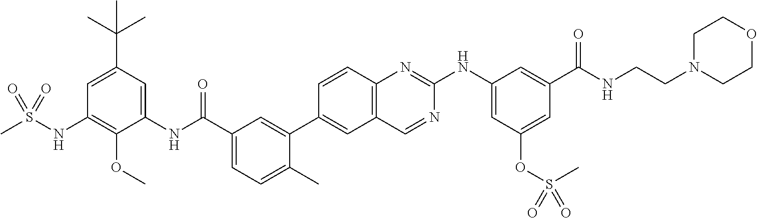 [SMILES: COc1c(NC(=O)c2ccc(C)c(-c3ccc4nc(Nc5cc(OS(C)(=O)=O)cc(C(=O)NCCN6CCOCC6)c5)ncc4c3)c2)cc(C(C)(C)C)cc1NS(C)(=O)=O]

### (i) Methyl 3-amino-5-((methylsulfonyl)oxy)benzoate

Methyl 3-amino-5-hydroxybenzoate (239 mg, 1.430 mmol) and triethylamine (239 μL, 1.716 mmol) were stirred in dichloromethane (15 mL). Methanesulfonyl chloride (122 μL, 1.573 mmol) was added and the mixture was stirred at room temperature for 18 h. The mixture was diluted with water (20 mL), and extracted with dichloromethane (3×40 mL). The combined organic phases were washed with saturated brine (20 mL), dried (MgSO₄) and concentrated to yield a colourless oil. The crude product was purified by chromatography on the Companion (12 g column, 0-50% EtOAc:iso-hexanes) to afford the sub-title compound (342 mg) as a colourless oil.

¹H NMR (DMSO-d6) 400 MHz, δ: 7.18 (t, 1H), 6.95 (t, 1H), 6.72 (t, 1H), 5.81 (s, 2H), 3.83 (s, 3H), 3.37 (s, 3H).

LCMS m/z 246 (M+H)⁺ (ES⁺)

### (ii) 3-Amino-5-((methylsulfonyl)oxy)benzoic acid

1M Sodium hydroxide solution (1.464 mL, 1.464 mmol) was added to a solution of the product from step (i) above (0.342 g, 1.394 mmol) in THF (1 mL) at rt then stirred for 4 h. The mixture was acidified with 1 M HCl (1.5 mL) and extracted with EtOAc (3×10 mL). The combined organic phases were washed with saturated brine (10 mL), dried (MgSO₄) and concentrated to yield the sub-title compound (287 mg).

¹H NMR (DMSO-d6) 400 MHz, δ:7.06 (t, 1H), 6.94 (t, 1H), 6.70 (t, 1H), 5.75 (brs, 2H), 3.36 (s, 3H).

LCMS m/z 232 (M+H)⁺ (ES⁺); 230 (M−H)⁻ (ES⁻)

### (iii) 3-Amino-5-((2-morpholinoethyl)carbamoyl)phenyl methanesulfonate

2-Morpholinoethanamine (0.261 mL, 1.986 mmol) was added to an ice cold suspension of T3P (50% in EtOAc, 0.739 mL, 1.241 mmol), the product from step (ii) above (0.287 g, 0.993 mmol) and TEA (0.415 mL, 2.98 mmol) in EtOAc (5 mL). The mixture was allowed to warm to room temperature and stir overnight. Sat. NaHCO₃ solution (20 mL) was added and the mixture was extracted with EtOAc (3×10 mL). The combined organic phases were washed with saturated brine (20 mL), dried (MgSO₄) and concentrated under reduced pressure to yield a sticky solid. The solid was triturated in diethyl ether to yield the sub-title compound (268 mg) as an off-white solid.

¹H NMR (400 MHz, DMSO) δ: 8.28 (t, 1H), 6.99 (t, 1H), 6.83 (t, 1H), 6.62 (t, 1H), 5.66 (br s, 2H), 3.61-3.53 (m, 4H), 3.37-3.30 (m, 2H), 3.36 (s, 3H), 2.49-2.38 (m, 6H).

LCMS m/z 344 (M+H)⁺ (ES⁺)

### (iv) 3-((6-(5-((5-(tert-Butyl)-2-methoxy-3-(methylsulfonamido)phenyl)carbamoyl)-2-methylphenyl)quinazolin-2-yl)amino)-5-((2-morpholinoethyl)carbamoyl)phenyl methanesulfonate

A solution of N-(5-(tert-butyl)-2-methoxy-3-(methylsulfonamido)phenyl)-4-methyl-3-(2-(methylsulfinyl)quinazolin-6-yl)benzamide (see Example 7(v) above; 80 mg, 0.138 mmol) and the product from step (iii) above (95 mg, 0.276 mmol) in dry DMF (1 mL) and treated with tosic acid (60 mg, 0.315 mmol). Reaction mixture was warmed to 70° C. and stirred for 24 h and then cooled to rt and stirred for 48 h. The reaction mixture was basified with aq. ammonia and purified by preparative HPLC (Waters, Basic (0.1% Ammonium Bicarbonate), Basic, Waters X-Bridge Prep-C18, 5 μm, 19×50 mm column, 35-65% MeCN in Water) and dried for 24 h under vacuum at 45° C. to afford the title compound (34 mg) as an off-white solid.

¹H NMR (400 MHz, DMSO-d6) δ 10.39 (s, 1H), 9.91 (s, 1H), 9.45 (s, 1H), 9.16 (s, 1H), 8.50 (s, 1H), 8.46 (d, 1H), 8.28 (t, 1H), 8.05 (d, 1H), 8.04-7.93 (m, 3H), 7.80 (d, 1H), 7.53 (d, 1H), 7.46 (d, 1H), 7.40-7.36 (m, 1H), 7.25 (d, 1H), 3.71 (s, 3H), 3.59 (t, 4H), 3.51 (s, 3H), 3.42 (d, 2H), 3.05 (s, 3H), 2.44 (s, 4H), 2.39 (s, 3H), 1.28 (s, 9H). 2 aliphatic signals obscured by DMSO peak.

LCMS m/z 860 (M+H)⁺ (ES⁺)

### Example 11

### 4-((6-(5-((5-(tert-Butyl)-2-methoxy-3-(methylsulfonamido)phenyl)carbamoyl)-2-methylphenyl)-quinazolin-2-yl)amino)-2-methoxy-N-(3-(1-oxidothiomorpholino)propyl)benzamide

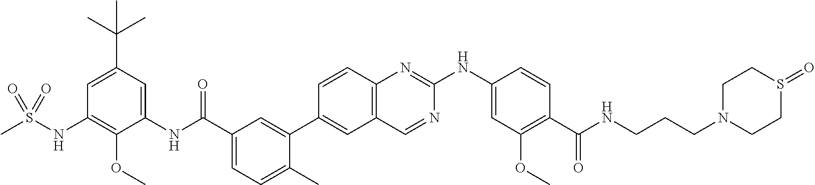 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCCN1CCS(=O)CC1]

A solution of 4-((6-(5-((5-(tert-butyl)-2-methoxy-3-(methylsulfonamido)phenyl)carbamoyl)-2-methylphenyl)quinazolin-2-yl)amino)-2-methoxybenzoic acid (see Example 8 above; 78 mg, 0.114 mmol) in dry DMF (0.5 mL) was treated with 4-(3-aminopropyl)thiomorpholine 1-oxide (22 mg, 0.125 mmol) and DIPEA (40 μL, 0.228 mmol). A solution of HATU (48 mg, 0.125 mmol) in dry DMF (0.5 mL) was added and the reaction mixture shaken at rt for 18 h. The crude product was purified by preparative HPLC (Waters, Basic (0.1% Ammonium Bicarbonate), Basic, Waters X-Bridge Prep-C18, 5 μm, 19×50 mm column, 35-65% MeCN in Water) to afford the title compound (12 mg) as a yellow solid.

¹H NMR (400 MHz, DMSO-d6) δ 10.26 (s, 1H), 9.90 (s, 1H), 9.44 (s, 1H), 9.15 (s, 1H), 8.23 (d, 1H), 8.09 (t, 1H), 8.04 (d, 1H), 8.03-7.92 (m, 3H), 7.84 (dd, 2H), 7.59-7.50 (m, 2H), 7.47 (d, 1H), 7.25 (d, 1H), 4.01 (s, 3H), 3.71 (s, 3H), 3.32 (t, 2H), 3.05 (s, 3H), 2.93-2.82 (m, 4H), 2.78-2.61 (m, 4H), 2.44 (t, 2H), 2.39 (s, 3H), 1.71 (t, 2H), 1.28 (s, 9H).

LCMS m/z 842 (M+H)⁺ (ES⁺)

### Example 12

### N-(5-(tert-Butyl)-2-methoxy-3-(methylsulfonamido)phenyl)-3-(2-((4-(1,1-dioxidothiomorpholine-4-carbonyl)-3-methoxyphenyl)amino)quinazolin-6-yl)-4-methylbenzamide

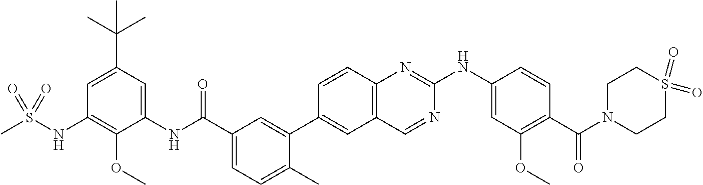 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)N1CCS(=O)(=O)CC1]

A solution of 4-((6-(5-((5-(tert-butyl)-2-methoxy-3-(methylsulfonamido)phenyl)carbamoyl)-2-methylphenyl)quinazolin-2-yl)amino)-2-methoxybenzoic acid (see Example 8 above; 78 mg, 0.114 mmol) in dry DMF (0.5 mL) was treated with thiomorpholine 1,1-dioxide (15 mg, 0.114 mmol) and DIPEA (40 μL, 0.228 mmol). A solution of HATU (48 mg, 0.125 mmol) in dry DMF (0.5 mL) was added and the reaction mixture shaken at rt for 18 h. The crude product was purified by preparative HPLC (Waters, Basic (0.1% Ammonium Bicarbonate), Basic, Waters X-Bridge Prep-C18, 5 μm, 19×50 mm column, 35-65% MeCN in Water) to afford the title compound (60 mg) as a yellow solid.

¹H NMR (400 MHz, DMSO-d6) δ 10.18 (s, 1H), 9.89 (s, 1H), 9.42 (s, 1H), 9.15 (s, 1H), 8.13 (d, 1H), 8.06-7.90 (m, 4H), 7.83 (d, 1H), 7.59-7.50 (m, 2H), 7.47 (d, 1H), 7.29 (d, 1H), 7.26 (d, 1H), 3.90 (s, 5H), 3.72 (s, 3H), 3.64 (s, 2H), 3.05 (s, 7H), 2.40 (s, 3H), 1.28 (s, 9H).

LCMS m/z 801 (M+H)⁺ (ES⁺)

### Example 13

### 4-((6-(5-((5-(tert-Butyl)-2-methoxy-3-(methylsulfonamido)phenyl)carbamoyl)-2-methylphenyl)-quinazolin-2-ylamino)-N-(2-(4-(2-hydroxyethyl)piperazin-1-yl)ethyl)-2-methoxybenzamide

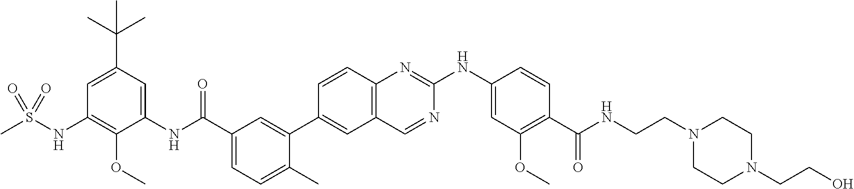 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCN1CCN(CCO)CC1]

A solution of 4-((6-(5-((5-(tert-butyl)-2-methoxy-3-(methylsulfonamido)phenyl)carbamoyl)-2-methylphenyl)quinazolin-2-yl)amino)-2-methoxybenzoic acid (see Example 8 above; 47 mg, 0.069 mmol), 2-(4-(2-aminoethyl)piperazin-1-yl)ethanol (13 mg, 0.076 mmol) and DIPEA (24 μL, 0.137 mmol) in dry DMF (0.3 mL) was treated with a solution of HATU (29 mg, 0.076 mmol) in dry DMF (0.3 mL) and the reaction mixture shaken at rt for 18 h. The crude product was purified by preparative HPLC (Waters, Basic (0.1% Ammonium Bicarbonate), Basic, Waters X-Bridge Prep-C18, 5 μm, 19×50 mm column, 35-65% MeCN in Water) to afford the title compound (39 mg) as a yellow solid.

¹H NMR (400 MHz, DMSO-d6) δ 10.28 (s, 1H), 9.88 (s, 1H), 9.44 (d, 1H), 9.15 (s, 1H), 8.32 (s, 1H), 8.26 (d, 1H), 8.08-7.81 (m, 6H), 7.59-7.50 (m, 2H), 7.47 (d, 1H), 7.25 (d, 1H), 4.38 (t, 1H), 4.05 (s, 3H), 3.71 (s, 3H), 3.51 (q, 2H), 3.41 (d, 2H), 3.05 (s, 3H), 2.48-2.37 (m, 15H), 1.28 (s, 9H). Aliphatics obscured under DMSO signal.

LCMS m/z 839 (M+H)⁺ (ES⁺); 837 (M−H)⁻ (ES⁻)

### Example 14

The following compounds were prepared by methods analogous to those described above. Where chemical shifts from 1H NMR spectra are reported, these were obtained at 400 MHz and ambient temperature, unless otherwise specified.

### (a) 3-((6-(5-((5-(tert-Butyl)-2-methoxy-3-(methylsulfonamido)phenyl)carbamoyl)-2-methylphenyl)quinazolin-2-yl)amino)phenyl methanesulfonate

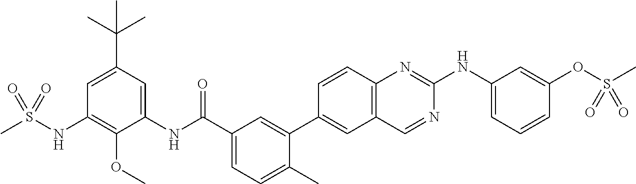

¹H NMR (400 MHz, DMSO-d6) δ 10.25 (s, 1H), 9.88 (s, 1H), 9.43 (d, 1H), 9.14 (s, 1H), 8.28 (t, 1H), 8.03 (d, 1H), 7.97 (dd, 3H), 7.88 (d, 1H), 7.80 (d, 1H), 7.53 (d, 1H), 7.51-7.42 (m, 2H), 7.25 (d, 1H), 6.98 (ddd, 1H), 3.71 (s, 3H), 3.45 (s, 3H), 3.04 (s, 3H), 2.40 (s, 3H), 1.28 (s, 9H).

LCMS m/z 704 (M+H)⁺ (ES⁺); 702 (M−H)⁻ (ES⁻)

### (b) N-(5-(tert-Butyl)-2-methoxy-3-(methylsulfonamido)phenyl)-3-(2-((3-cyano-5-(2-morpholinoethoxy)phenyl)amino)quinazolin-6-yl)-4-methylbenzamide

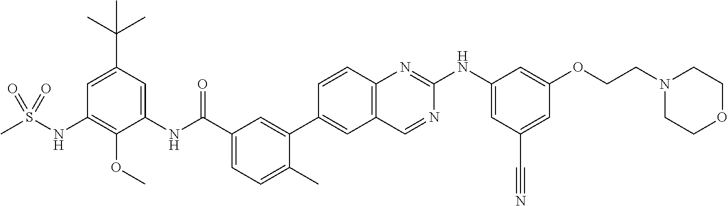 [SMILES: COc1c(NC(=O)c2ccc(C)c(-c3ccc4nc(Nc5cc(C#N)cc(OCCN6CCOCC6)c5)ncc4c3)c2)cc(C(C)(C)C)cc1NS(C)(=O)=O]

¹H NMR (400 MHz, DMSO-d6) δ 10.30 (s, 1H), 9.89 (s, 1H), 9.45 (d, 1H), 9.14 (s, 1H), 8.08-8.03 (m, 2H), 8.03-7.92 (m, 4H), 7.84 (d, 1H), 7.53 (d, 1H), 7.47 (d, 1H), 7.25 (d, 1H), 7.09 (dd, 1H), 4.20 (t, 2H), 3.71 (s, 3H), 3.62-3.58 (m, 4H), 3.05 (s, 3H), 2.75 (t, 2H), 2.39 (s, 3H), 1.28 (s, 9H). 4 protons under DMSO signal.

LCMS m/z 764 (M+H)⁺ (ES⁺)

### (c) N-(5-(tert-Butyl)-2-methoxy-3-(methylsulfonamido)phenyl)-3-(2-((3-methoxy-5-(N-methyl-N-(2-morpholinoethyl)sulfamoyl)phenyl)amino)quinazolin-6-yl)-4-methylbenzamide

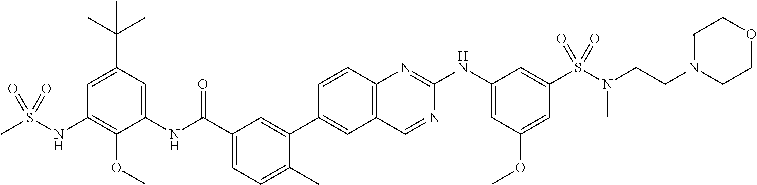 [SMILES: COc1cc(Nc2ncc3cc(-c4cc(C(=O)Nc5cc(C(C)(C)C)cc(NS(C)(=O)=O)c5OC)ccc4C)ccc3n2)cc(S(=O)(=O)N(C)CCN2CCOCC2)c1]

¹H NMR (400 MHz, DMSO-d6) δ 10.30 (s, 1H), 9.89 (s, 1H), 9.44 (d, 1H), 9.14 (s, 1H), 8.18 (t, 1H), 8.05 (d, 1H), 8.04-7.91 (m, 4H), 7.76 (d, 1H), 7.53 (d, 1H), 7.47 (d, 1H), 7.25 (d, 1H), 6.89 (dd, 1H), 3.88 (s, 3H), 3.71 (s, 3H), 3.56-3.51 (m, 4H), 3.19 (t, 2H), 3.05 (s, 3H), 2.83 (s, 3H), 2.39 (s, 7H), 1.28 (s, 9H). 2 protons under DMSO signal.

LCMS m/z 846 (M+H)⁺ (ES⁺)

### (d) N-(5-(tert-Butyl)-2-methoxy-3-(methylsulfonamido)phenyl)-4-methyl-3-(2-((3-((2-morpholinoethyl)carbamoyl)phenyl)amino)quinazolin-6-yl)benzamide

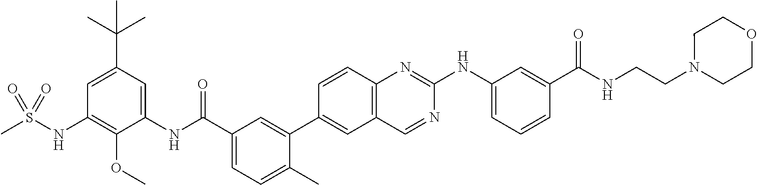 [SMILES: COc1c(NC(=O)c2ccc(C)c(-c3ccc4nc(Nc5cccc(C(=O)NCCN6CCOCC6)c5)ncc4c3)c2)cc(C(C)(C)C)cc1NS(C)(=O)=O]

¹H NMR (400 MHz, DMSO-d6) δ 10.08 (s, 1H), 9.89 (s, 1H), 9.40 (d, 1H), 9.14 (s, 1H), 8.42 (s, 1H), 8.32 (d, 1H), 8.21-8.13 (m, 1H), 8.04-7.97 (m, 2H), 7.98-7.90 (m, 2H), 7.77 (d, 1H), 7.52 (d, 1H), 7.48-7.42 (m, 3H), 7.25 (d, 1H), 3.71 (s, 3H), 3.59 (t, 4H), 3.41 (m, 2H), 3.05 (s, 3H), 2.46 (d, 6H), 2.40 (s, 3H), 1.28 (s, 9H).

LCMS m/z 766 (M+H)⁺ (ES⁺)

### (e) N-(5-(tert-Butyl)-2-methoxy-3-(methylsulfonamido)phenyl)-3-(2-((3-methoxy-5-(N-methyl-N-(3-morpholinopropyl)sulfamoyl)phenyl)amino)quinazolin-6-yl)-4-methylbenzamide

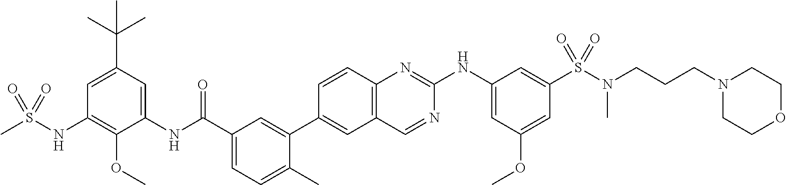 [SMILES: COc1cc(Nc2ncc3cc(-c4cc(C(=O)Nc5cc(C(C)(C)C)cc(NS(C)(=O)=O)c5OC)ccc4C)ccc3n2)cc(S(=O)(=O)N(C)CCCN2CCOCC2)c1]

¹H NMR (400 MHz, DMSO-d6) δ 10.31 (s, 1H), 9.89 (s, 1H), 9.44 (d, 1H), 9.14 (s, 1H), 8.18 (t, 1H), 8.05 (d, 1H), 8.02-7.92 (m, 4H), 7.76 (d, 1H), 7.53 (d, 1H), 7.47 (d, 1H), 7.25 (d, 1H), 6.87 (dd, 1H), 3.88 (s, 3H), 3.71 (s, 3H), 3.52 (m, 4H), 3.08 (t, 2H), 3.05 (s, 3H), 2.78 (s, 3H), 2.39 (s, 3H), 2.29 (t, 6H), 1.71-1.61 (m, 2H), 1.28 (s, 9H).

LCMS m/z 860 (M+H)⁺ (ES⁺)

### (f) N-(5-(tert-Butyl)-2-methoxy-3-(methylsulfonamido)phenyl)-3-(2-((3-cyano-5-(2-(2-(2-methoxyethoxy)ethoxy)ethoxy)phenyl)amino)quinazolin-6-yl)-4-methylbenzamide

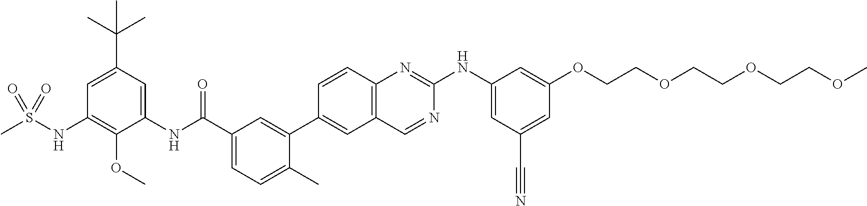

¹H NMR (400 MHz, DMSO-d6) δ 10.31 (s, 1H), 9.88 (s, 1H), 9.46 (d, 1H), 9.14 (s, 1H), 8.07-8.02 (m, 3H), 7.97 (ddd, 3H), 7.87-7.83 (m, 1H), 7.53 (d, 1H), 7.46 (d, 1H), 7.25 (d, 1H), 7.08 (dd, 1H), 4.25-4.17 (m, 2H), 3.83-3.77 (m, 2H), 3.71 (s, 3H), 3.65-3.59 (m, 2H), 3.59-3.50 (m, 4H), 3.47-3.40 (m, 2H), 3.23 (s, 3H), 3.04 (s, 3H), 2.39 (s, 3H), 1.28 (s, 9H).

LCMS m/z 797 (M+H)⁺ (ES⁺)

### (g) N-(5-(tert-Butyl)-2-methoxy-3-(methylsulfonamido)phenyl)-3-(2-((3-ethynyl-5-((2-(2-(2-methoxyethoxy)ethoxy)ethyl)carbamoyl)phenyl)amino)quinazolin-6-yl)-4-methylbenzamide

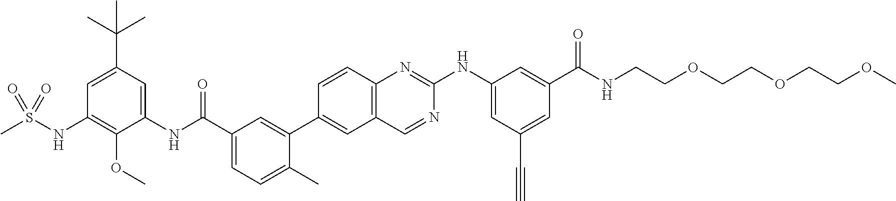

¹H NMR (400 MHz, DMSO-d6) δ 10.22 (s, 1H), 9.89 (s, 1H), 9.43 (d, 1H), 9.14 (s, 1H), 8.56 (s, 1H), 8.51 (t, 1H), 8.30 (dd, 1H), 8.04 (d, 1H), 8.02-7.91 (m, 3H), 7.78 (dt, 1H), 7.58 (t, 1H), 7.53 (d, 1H), 7.46 (d, 1H), 7.25 (d, 1H), 4.27 (s, 1H), 3.71 (s, 3H), 3.61-3.49 (m, 8H), 3.48-3.38 (m, 4H), 3.22 (s, 3H), 3.05 (s, 3H), 2.40 (s, 3H), 1.28 (s, 9H).

LCMS m/z 823 (M+H)⁺ (ES⁺)

### (h) N-(5-(tert-Butyl)-2-methoxy-3-(methylsulfonamido)phenyl)-3-(2-((3-(dimethylphosphoryl)phenyl)amino)quinazolin-6-yl)-4-methylbenzamide

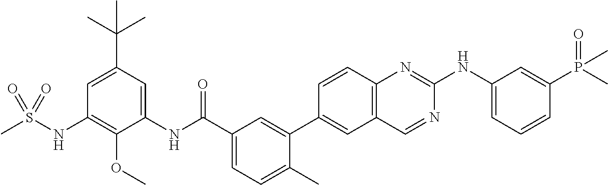

¹H NMR (400 MHz, DMSO-d6) δ 10.13 (s, 1H), 9.89 (s, 1H), 9.41 (d, 1H), 9.15 (s, 1H), 8.46 (dt, 1H), 8.21-8.13 (m, 1H), 8.04-7.90 (m, 4H), 7.76 (d, 1H), 7.56-7.44 (m, 3H), 7.38 (ddt, 1H), 7.25 (d, 1H), 3.71 (s, 3H), 3.05 (s, 3H), 2.40 (s, 3H), 1.70 (d, 6H), 1.28 (s, 9H).

LCMS m/z 686 (M+H)⁺ (ES⁺)

### (i) N-(5-(tert-Butyl)-2-methoxy-3-(methylsulfonamido)phenyl)-3-(2-((3-((dimethylphosphoryl)methyl)phenyl)amino)quinazolin-6-yl)-4-methylbenzamide

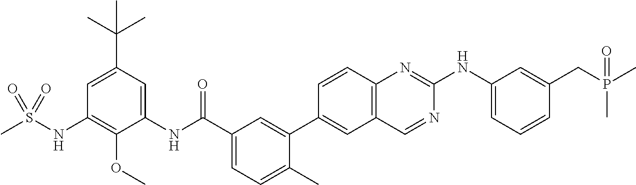

¹H NMR (400 MHz, DMSO-d6) δ 9.93 (s, 1H), 9.89 (s, 1H), 9.37 (d, 1H), 9.14 (s, 1H), 8.03-7.87 (m, 6H), 7.77 (d, 1H), 7.52 (d, 1H), 7.46 (d, 1H), 7.28-7.17 (m, 3H), 3.71 (s, 3H), 3.11 (d, 2H), 3.05 (s, 3H), 2.40 (s, 3H), 1.35 (d, 6H), 1.28 (s, 9H).

LCMS m/z 700 (M+H)⁺ (ES⁺)

### (j) 4-((6-(5-((5-(tert-Butyl)-2-methoxy-3-(methylsulfonamido)phenyl)carbamoyl)-2-methylphenyl)quinazolin-2-yl)amino)phenyl methanesulfonate

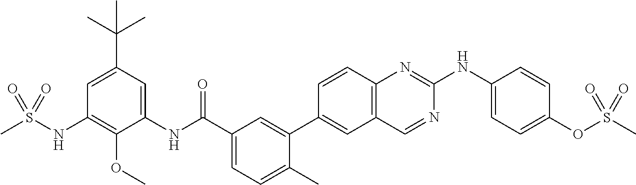

¹H NMR (400 MHz, DMSO-d6) δ 10.16 (s, 1H), 9.88 (s, 1H), 9.41 (d, 1H), 9.15 (s, 1H), 8.15-8.08 (m, 2H), 8.05-7.91 (m, 4H), 7.84-7.74 (m, 1H), 7.52 (d, 1H), 7.46 (d, 1H), 7.37-7.32 (m, 2H), 7.25 (d, 1H), 3.71 (s, 3H), 3.37 (s, 3H), 3.05 (s, 3H), 2.40 (s, 3H), 1.28 (s, 9H).

LCMS m/z 704 (M+H)⁺ (ES⁺)

### (k) N-(5-(tert-Butyl)-2-methoxy-3-(methylsulfonamido)phenyl)-3-(2-((3-ethynyl-5-(2-(2-(2-methoxyethoxy)ethoxy)ethoxy)phenyl)amino)quinazolin-6-yl)-4-methylbenzamide

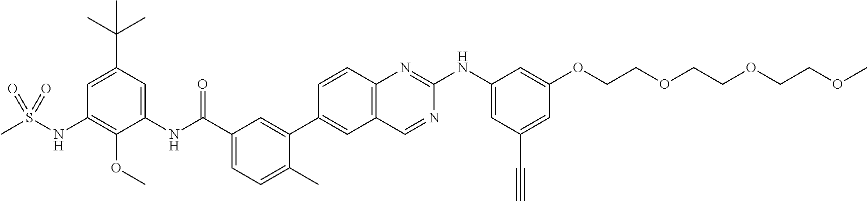

¹H NMR (400 MHz, DMSO-d6) δ 10.07 (s, 1H), 9.89 (s, 1H), 9.41 (d, 1H), 9.14 (s, 1H), 8.05-7.98 (m, 2H), 7.95 (ddd, 2H), 7.88 (t, 1H), 7.79 (d, 1H), 7.67 (s, 1H), 7.52 (d, 1H), 7.46 (d, 1H), 7.25 (d, 1H), 6.69 (dd, 1H), 4.16 (m, 3H), 3.82-3.76 (m, 2H), 3.71 (s, 3H), 3.65-3.60 (m, 2H), 3.59-3.51 (m, 4H), 3.48-3.40 (m, 2H), 3.23 (s, 3H), 3.05 (s, 3H), 2.39 (s, 3H), 1.28 (s, 9H).

LCMS m/z 796 (M+H)⁺ (ES⁺)

### (l) N-(5-(tert-Butyl)-2-methoxy-3-(methylsulfonamido)phenyl)-3-(2-((3-fluoro-5-(2-(2-(2-methoxyethoxy)ethoxy)ethoxy)phenyl)amino)quinazolin-6-yl)-4-methylbenzamide

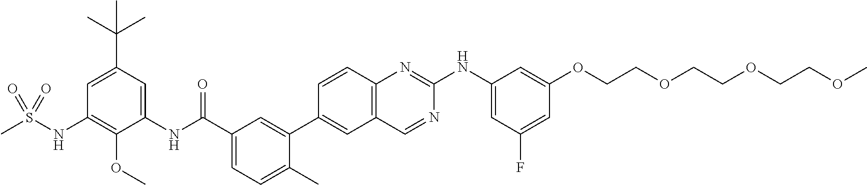

¹H NMR (400 MHz, DMSO-d6) δ 10.15 (s, 1H), 9.88 (s, 1H), 9.42 (d, 1H), 9.14 (s, 1H), 8.04-7.98 (m, 2H), 7.95 (dd, 2H), 7.82 (d, 1H), 7.61 (d, 1H), 7.55-7.49 (m, 1H), 7.46 (d, 2H), 7.25 (d, 1H), 6.48 (d, 1H), 4.17-4.10 (m, 2H), 3.78 (t, 2H), 3.71 (s, 3H), 3.62 (dd, 2H), 3.60-3.51 (m, 4H), 3.47-3.40 (m, 2H), 3.24 (s, 3H), 3.04 (s, 3H), 2.39 (s, 3H), 1.28 (s, 9H).

LCMS m/z 790 (M+H)⁺ (ES⁺)

### (m) N-(5-(tert-Butyl)-2-methoxy-3-(methylsulfonamido)phenyl)-3-(2-((3-ethynyl-5-((2-(2-methoxyethoxy)ethyl)carbamoyl)phenyl)amino)quinazolin-6-yl)-4-methylbenzamide

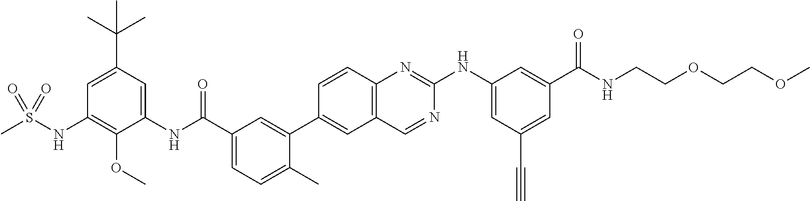

¹H NMR (400 MHz, DMSO-d6) δ 10.22 (s, 1H), 9.88 (s, 1H), 9.43 (d, 1H), 9.15 (s, 1H), 8.56 (t, 1H), 8.50 (t, 1H), 8.32-8.28 (m, 1H), 8.04 (d, 1H), 8.02-7.90 (m, 3H), 7.78 (d, 1H), 7.58 (t, 1H), 7.53 (d, 1H), 7.46 (d, 1H), 7.25 (d, 1H), 4.27 (s, 1H), 3.71 (s, 3H), 3.60-3.53 (m, 4H), 3.50-3.39 (m, 4H), 3.25 (s, 3H), 3.04 (s, 3H), 2.40 (s, 3H), 1.28 (s, 9H).

LCMS m/z 779 (M+H)⁺ (ES⁺)

### (n) N-(5-(tert-Butyl)-2-methoxy-3-(methylsulfonamido)phenyl)-3-(2-((3-ethynyl-5-((2-methoxyethyl)carbamoyl)phenyl)amino)quinazolin-6-yl)-4-methylbenzamide

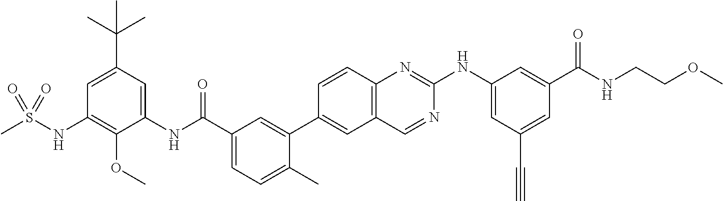

¹H NMR (400 MHz, DMSO-d6) δ 10.22 (s, 1H), 9.89 (s, 1H), 9.43 (d, 1H), 9.14 (s, 1H), 8.57 (t, 1H), 8.51 (t, 1H), 8.31 (s, 1H), 8.04 (d, 1H), 8.02-7.92 (m, 3H), 7.78 (d, 1H), 7.58 (d, 1H), 7.53 (d, 1H), 7.46 (d, 1H), 7.25 (d, 1H), 4.27 (s, 1H), 3.71 (s, 3H), 3.52-3.42 (m, 4H), 3.30 (s, 3H), 3.04 (s, 3H), 2.40 (s, 3H), 1.28 (s, 9H).

LCMS m/z 735 (M+H)⁺ (ES⁺)

### (o) 3-((6-(5-((5-(tert-Butyl)-2-methoxy-3-(methylsulfonamido)phenyl)carbamoyl)-2-methylphenyl)quinazolin-2-yl)amino)-5-methoxybenzoic acid

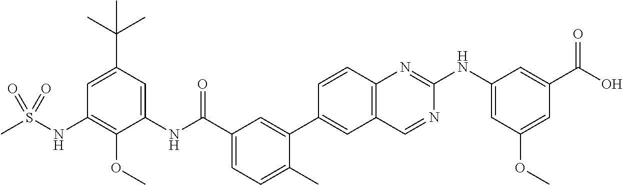 [SMILES: COc1cc(Nc2ncc3cc(-c4cc(C(=O)Nc5cc(C(C)(C)C)cc(NS(C)(=O)=O)c5OC)ccc4C)ccc3n2)cc(C(=O)O)c1]

¹H NMR (400 MHz, DMSO-d6) δ 12.92 (s, 1H), 10.16 (s, 1H), 9.94 (s, 1H), 9.42 (d, 1H), 9.17 (s, 1H), 8.17-8.09 (m, 2H), 8.05-7.91 (m, 4H), 7.80-7.74 (m, 1H), 7.52 (d, 1H), 7.46 (d, 1H), 7.25 (d, 1H), 7.11 (dd, 1H), 3.85 (s, 3H), 3.71 (s, 3H), 3.05 (s, 3H), 2.39 (s, 3H), 1.27 (s, 9H).

LCMS m/z 684 (M+H)⁺ (ES⁺); 682 (M−H)⁻ (ES⁻)

### (p) 4-((6-(5-((5-(tert-Butyl)-2-methoxy-3-(methylsulfonamido)phenyl)carbamoyl)-2-methylphenyl)quinazolin-2-yl)amino)-2-methoxy-N-(2-(2-(2-methoxyethoxy)ethoxy)ethyl)benzamide

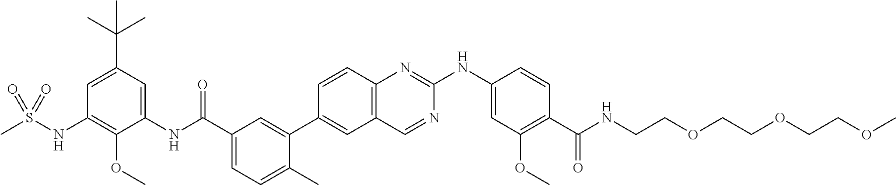

¹H NMR (400 MHz, DMSO-d6) δ 10.28 (s, 1H), 9.86 (s, 1H), 9.43 (s, 1H), 9.13 (s, 1H), 8.24 (d, 1H), 8.19 (dd, 1H), 8.03 (d, 1H), 7.98-7.94 (m, 3H), 7.88 (d, 1H), 7.84 (d, 1H), 7.55-7.51 (m, 2H), 7.45 (s, 1H), 7.24 (s, 1H), 4.01 (s, 3H), 3.70 (s, 3H), 3.58-3.42 (m, 12H), 3.23 (s, 3H), 3.02 (bs, 3H), 2.39 (s, 3H), 1.27 (s, 9H).

LCMS m/z 829 (M+H)⁺ (ES⁺)

### (q) N-(5-(tert-Butyl)-2-methoxy-3-(methylsulfonamido)phenyl)-3-(2-((3-((dimethyl(oxo)-I6-sulfanylidene)amino)phenyl)amino)quinazolin-6-yl)-4-methylbenzamide

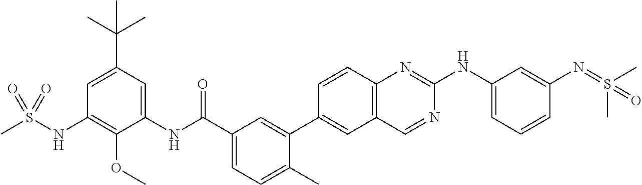 [SMILES: COc1c(NC(=O)c2ccc(C)c(-c3ccc4nc(Nc5cccc(N=S(C)(C)=O)c5)ncc4c3)c2)cc(C(C)(C)C)cc1NS(C)(=O)=O]

¹H NMR (400 MHz, DMSO-d6) δ 9.92 (s, 1H), 9.85 (s, 1H), 9.36 (s, 1H), 7.99 (s, 1H), 7.99-7.89 (m, 3H), 7.83 (s, 1H), 7.71 (d, 1H), 7.52 (d, 1H), 7.44 (d, 2H), 7.25 (d, 1H), 7.13 (t, 1H), 6.58 (d, 1H), 3.70 (s, 3H), 3.29 (s, 6H), 3.04 (s, 3H), 2.39 (s, 3H), 1.27 (s, 9H).

LCMS m/z 701 (M+H)⁺ (ES⁺)

### (r) N-(5-(tert-Butyl)-2-methoxy-3-(methylsulfonamido)phenyl)-3-(2-((3-(2-(2-(2-methoxy-ethoxy)ethoxy)ethoxy)-5-(trifluoromethoxy)phenyl)amino)quinazolin-6-yl)-4-methylbenzamide

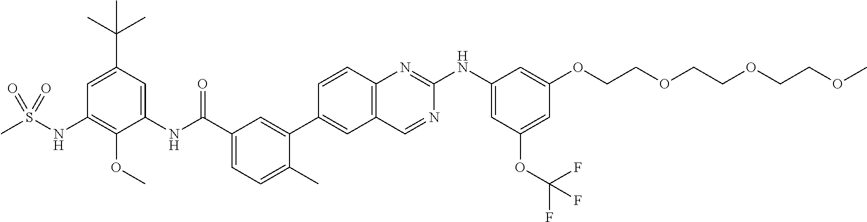

¹H NMR (400 MHz, DMSO-d6) δ 10.24 (s, 1H), 9.90 (s, 1H), 9.44 (d, 1H), 9.14 (s, 1H), 8.04 (d, 1H), 8.02-7.92 (m, 3H), 7.86-7.76 (m, 2H), 7.70 (t, 1H), 7.53 (d, 1H), 7.47 (d, 1H), 7.25 (d, 1H), 6.58 (s, 1H), 4.17 (t, 2H), 3.80 (dd, 2H), 3.71 (s, 3H), 3.65-3.60 (m, 2H), 3.59-3.50 (m, 4H), 3.47-3.41 (m, 2H), 3.23 (s, 3H), 3.05 (s, 3H), 2.39 (s, 3H), 1.28 (s, 9H).

LCMS m/z 856 (M+H)⁺ (ES⁺)

### (s) N-(5-(tert-Butyl)-2-methoxy-3-(methylsulfonamido)phenyl)-3-(2-((3-(2-(2-(2-methoxyethoxy)ethoxy)ethoxy)-5-(trifluoromethyl)phenyl)amino)quinazolin-6-yl)-4-methylbenzamide

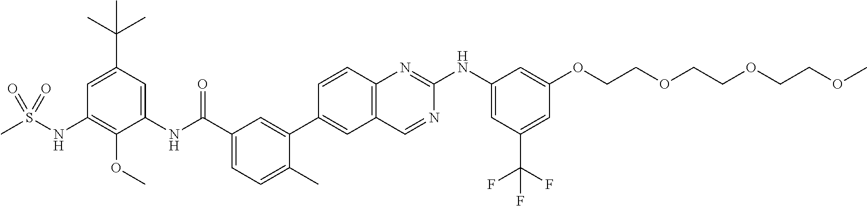

¹H NMR (400 MHz, DMSO-d6) δ 10.30 (s, 1H), 9.90 (s, 1H), 9.45 (d, 1H), 9.14 (s, 1H), 8.05 (d, 2H), 8.03-7.91 (m, 4H), 7.80 (d, 1H), 7.53 (d, 1H), 7.47 (d, 1H), 7.25 (d, 1H), 6.89 (s, 1H), 4.23 (t, 2H), 3.84-3.78 (m, 2H), 3.71 (s, 3H), 3.66-3.59 (m, 2H), 3.59-3.49 (m, 4H), 3.45-3.40 (m, 2H), 3.23 (s, 3H), 3.05 (s, 3H), 2.39 (s, 3H), 1.28 (s, 9H).

LCMS m/z 840 (M+H)⁺ (ES⁺)

### (t) N-(5-(tert-Butyl)-2-methoxy-3-(methylsulfonamido)phenyl)-4-methyl-3-(2-((4-(S-methylsulfonimidoyl)phenyl)amino)quinazolin-6-yl)benzamide

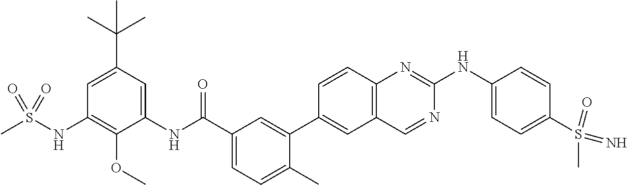 [SMILES: COc1c(NC(=O)c2ccc(C)c(-c3ccc4nc(Nc5ccc(S(C)(=N)=O)cc5)ncc4c3)c2)cc(C(C)(C)C)cc1NS(C)(=O)=O]

¹H NMR (400 MHz, DMSO-d6) δ 9.90 (s, 1H), 9.30 (d, 1H), 8.00-7.90 (m, 3H), 7.90-7.83 (m, 1H), 7.65 (dd, 3H), 7.50 (d, 1H), 7.44 (d, 1H), 7.24 (d, 1H), 6.69-6.62 (m, 2H), 6.11 (s, 2H), 3.69 (s, 3H), 3.50 (s, 3H), 3.04 (s, 3H), 2.36 (s, 3H), 1.27 (s, 9H).

LCMS m/z 687 (M+H)+ (ES+)

### (u) N-(5-(tert-Butyl)-2-methoxy-3-(methylsulfonamido)phenyl)-4-methyl-3-(2-((3-((2-morpholinoethyl)carbamoyl)-5-(trifluoromethyl)phenyl)amino)quinazolin-6-yl)benzamide

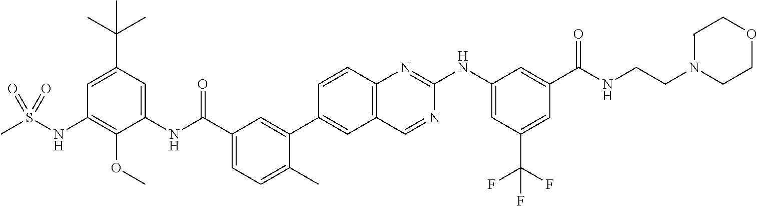 [SMILES: COc1c(NC(=O)c2ccc(C)c(-c3ccc4nc(Nc5cc(C(=O)NCCN6CCOCC6)cc(C(F)(F)F)c5)ncc4c3)c2)cc(C(C)(C)C)cc1NS(C)(=O)=O]

¹H NMR (400 MHz, DMSO-d6) δ 10.49 (s, 1H), 9.91 (s, 1H), 9.47 (d, 1H), 9.16 (s, 1H), 8.71 (s, 1H), 8.71-8.60 (m, 2H), 8.07 (d, 1H), 8.04-7.92 (m, 3H), 7.79 (d, 2H), 7.53 (d, 1H), 7.46 (d, 1H), 7.25 (d, 1H), 3.71 (s, 3H), 3.59 (t, 4H), 3.44 (d, 2H), 3.05 (s, 3H), 2.44 (d, 4H), 2.40 (s, 3H), 1.28 (s, 9H). 2 aliphatic signals obscured by DMSO signal.

LCMS m/z 834 (M+H)⁺ (ES⁺)

### (v) 4-((6-(5-((5-(tert-Butyl)-2-methoxy-3-(methylsulfonamido)phenyl)carbamoyl)-2-methylphenyl)quinazolin-2-yl)amino)-N-(2-(2-(2-hydroxyethoxy)ethoxy)ethyl)-2-methoxybenzamide

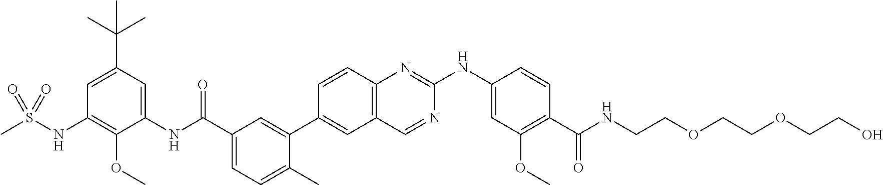 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCOCCOCCO]

¹H NMR (400 MHz, DMSO-d6) δ 10.29 (s, 1H), 9.89 (s, 1H), 9.44 (s, 1H), 9.15 (s, 1H), 8.26 (d, 1H), 8.21 (t, 1H), 8.04 (d, 1H), 8.03-7.92 (m, 3H), 7.88 (dd, 2H), 7.57-7.50 (m, 2H), 7.47 (d, 1H), 7.25 (d, 1H), 4.60 (t, 1H), 4.02 (s, 3H), 3.72 (s, 3H), 3.63-3.43 (m, 12H), 3.05 (s, 3H), 2.40 (s, 3H), 1.28 (s, 9H).

LCMS m/z 815 (M+H)⁺ (ES⁺)

### (w) 4-((6-(5-((5-(tert-Butyl)-2-methoxy-3-(methylsulfonamido)phenyl)carbamoyl)-2-methylphenyl)quinazolin-2-yl)amino)-2-methoxy-N-(2-(2-methoxyethoxy)ethyl)benzamide

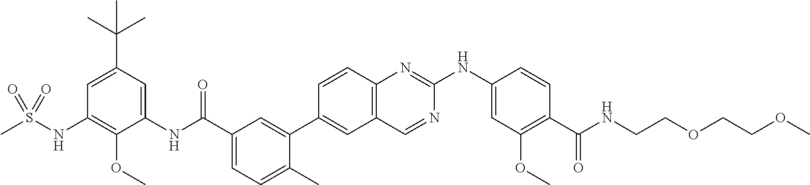

¹H NMR (400 MHz, DMSO-d6) δ 10.29 (s, 1H), 9.89 (s, 1H), 9.44 (s, 1H), 9.15 (s, 1H), 8.26 (d, 1H), 8.21 (t, 1H), 8.04 (d, 1H), 8.03-7.93 (m, 3H), 7.88 (dd, 2H), 7.57-7.50 (m, 2H), 7.47 (d, 1H), 7.25 (d, 1H), 4.02 (s, 3H), 3.72 (s, 3H), 3.63-3.52 (m, 4H), 3.52-3.43 (m, 4H), 3.28 (s, 3H), 3.05 (s, 3H), 2.39 (s, 3H), 1.28 (s, 9H).

LCMS m/z 785 (M+H)⁺ (ES⁺)

### (x) 4-((6-(5-((5-(tert-Butyl)-2-methoxy-3-(methylsulfonamido)phenyl)carbamoyl)-2-methylphenyl)quinazolin-2-yl)amino)-N-(3-hydroxy-2,2-bis(hydroxymethyl)propyl)-2-methoxybenzamide

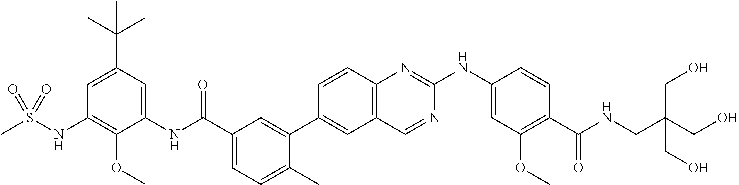 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCC(CO)(CO)CO]

¹H NMR (400 MHz, DMSO-d6) δ 10.29 (s, 1H), 9.88 (s, 1H), 9.44 (s, 1H), 9.15 (s, 1H), 8.60 (t, 1H), 8.26 (d, 1H), 8.04 (d, 1H), 8.02-7.93 (m, 3H), 7.88 (dd, 2H), 7.57-7.50 (m, 2H), 7.46 (d, 1H), 7.25 (d, 1H), 4.54 (t, 3H), 4.00 (s, 3H), 3.72 (s, 3H), 3.36 (d, 6H), 3.34 (d, 2H), 3.04 (s, 3H), 2.39 (s, 3H), 1.28 (s, 9H).

LCMS m/z 801 (M+H)⁺ (ES⁺)

### (y) N-(2-(1,4-Oxazepan-4-yl)ethyl)-4-((6-(5-((5-(tert-butyl)-2-methoxy-3-(methylsulfonamido)-phenyl)carbamoyl)-2-methylphenyl)quinazolin-2-yl)amino)-2-methoxybenzamide

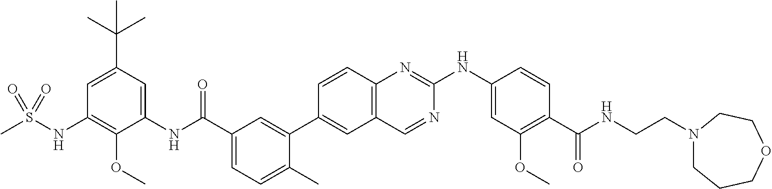 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCN1CCCOCC1]

¹H NMR (400 MHz, DMSO-d6) δ 10.28 (s, 1H), 9.90 (s, 1H), 9.44 (s, 1H), 9.15 (s, 1H), 8.31 (t, 1H), 8.25 (d, 1H), 8.04 (d, 1H), 8.02-7.94 (m, 3H), 7.91 (d, 1H), 7.86 (d, 1H), 7.59-7.51 (m, 2H), 7.48 (d, 1H), 7.26 (d, 1H), 4.04 (s, 3H), 3.71 (m, 5H), 3.69-3.64 (m, 2H), 3.40 (q, 2H), 3.06 (s, 3H), 2.71 (dt, 4H), 2.66 (t, 2H), 2.40 (s, 3H), 1.84 (p, 2H), 1.28 (s, 9H).

LCMS m/z 810 (M+H)⁺ (ES⁺)

### (z) 4-((6-(5-((5-(tert-Butyl)-2-methoxy-3-(methylsulfonamido)phenyl)carbamoyl)-2-methyl-phenyl)quinazolin-2-yl)amino)-2-methoxy-N-(2-(4-methylpiperazin-1-yl)ethyl)benzamide

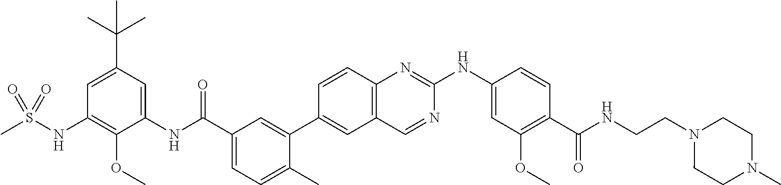

¹H NMR (400 MHz, DMSO-d6) δ 10.29 (s, 1H), 9.89 (s, 1H), 9.44 (s, 1H), 9.16 (s, 1H), 8.31 (t, 1H), 8.26 (d, 1H), 8.06-7.84 (m, 6H), 7.58-7.50 (m, 2H), 7.48 (d, 1H), 7.26 (d, 1H), 4.05 (s, 3H), 3.72 (s, 3H), 3.41 (q, 2H), 3.06 (s, 3H), 2.44 (m, 13H), 2.19 (s, 3H), 1.28 (s, 9H).

LCMS m/z 809 (M+H)⁺ (ES⁺)

### (aa) 4-((6-(5-((5-(tert-Butyl)-2-methoxy-3-(methylsulfonamido)phenyl)carbamoyl)-2-methyl-phenyl)quinazolin-2-yl)amino)-2-methoxy-N-(2-(1-methylpiperidin-4-yl)ethyl)benzamide

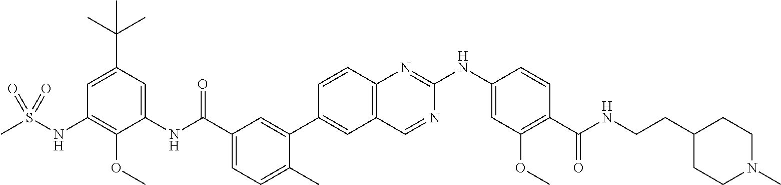

¹H NMR (400 MHz, DMSO-d6) δ 10.26 (s, 1H), 9.89 (s, 1H), 9.44 (d, 1H), 9.19 (s, 1H), 8.23 (d, 1H), 8.08-7.91 (m, 5H), 7.84 (t, 2H), 7.58-7.49 (m, 2H), 7.47 (d, 1H), 7.26 (d, 1H), 4.00 (s, 3H), 3.72 (s, 3H), 3.33 (dt, 2H), 3.05 (s, 3H), 2.74 (dt, 2H), 2.39 (s, 3H), 2.13 (s, 3H), 1.82 (td, 2H), 1.66 (dd, 2H), 1.47 (q, 2H), 1.28 (s, 12H).

LCMS m/z 808 (M+H)⁺ (ES⁺)

### (ab) N-(3-(1,4-Oxazepan-4-yl)propyl)-4-((6-(5-((5-(tert-butyl)-2-methoxy-3-(methyl-sulfonamido)phenyl)carbamoyl)-2-methylphenyl)quinazolin-2-yl)amino)-2-methoxybenzamide

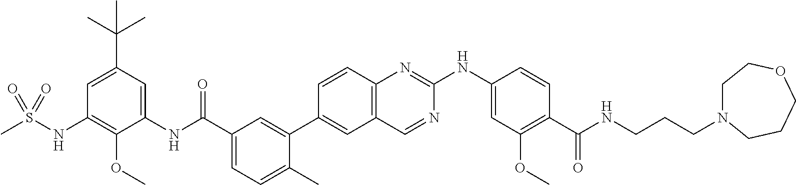 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCCN1CCCOCC1]

¹H NMR (400 MHz, DMSO-d6) δ 10.26 (s, 1H), 9.89 (s, 1H), 9.46-9.42 (m, 1H), 9.15 (s, 1H), 8.23 (d, 1H), 8.13-7.91 (m, 5H), 7.84 (dd, 2H), 7.58-7.49 (m, 2H), 7.48 (d, 1H), 7.26 (d, 1H), 4.01 (s, 3H), 3.72 (s, 3H), 3.67 (t, 2H), 3.64-3.58 (m, 2H), 3.34 (q, 2H), 3.06 (s, 3H), 2.66-2.60 (m, 3H), 2.54-2.48 (m, 3H), 2.39 (s, 3H), 1.80 (p, 2H), 1.68 (p, 2H), 1.28 (s, 9H).

LCMS m/z 824 (M+H)⁺ (ES⁺)

### (ac) 4-((6-(5-((5-(tert-Butyl)-2-methoxy-3-(methylsulfonamido)phenyl)carbamoyl)-2-methylphenyl)quinazolin-2-yl)amino)-2-methoxy-N-(1-methylpiperidin-4-yl)benzamide

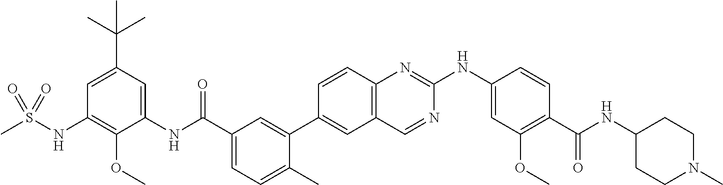

¹H NMR (400 MHz, DMSO-d6) δ 10.27 (s, 1H), 9.89 (s, 1H), 9.44 (s, 1H), 9.16 (s, 1H), 8.25 (d, 1H), 8.06-7.93 (m, 4H), 7.90 (d, 1H), 7.84 (dd, 2H), 7.58-7.50 (m, 2H), 7.48 (d, 1H), 7.26 (d, 1H), 4.02 (s, 3H), 3.87-3.75 (m, 1H), 3.72 (s, 3H), 3.05 (s, 3H), 2.66 (d, 2H), 2.39 (s, 3H), 2.18 (s, 3H), 2.14-2.00 (m, 2H), 1.89-1.79 (m, 2H), 1.64-1.50 (m, 2H), 1.28 (s, 9H).

LCMS m/z 780 (M+H)⁺ (ES⁺)

### (ad) 4-((6-(5-((5-(tert-Butyl)-2-methoxy-3-(methylsulfonamido)phenyl)carbamoyl)-2-methylphenyl)quinazolin-2-yl)amino)-2-methoxy-N-(2-(methylsulfonyl)ethyl)benzamide

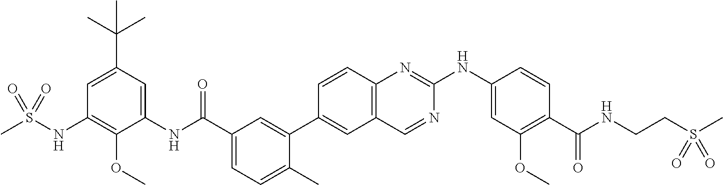

¹H NMR (400 MHz, DMSO-d6) δ 10.31 (s, 1H), 9.89 (s, 1H), 9.44 (d, 1H), 9.15 (s, 1H), 8.53 (d, 1H), 8.26 (d, 1H), 8.06-7.84 (m, 6H), 7.59-7.50 (m, 2H), 7.47 (d, 1H), 7.25 (d, 1H), 4.01 (s, 3H), 3.77 (t, 2H), 3.42 (t, J=6.6 Hz, 2H), 3.34 (s, 3H), 3.08 (s, 3H), 3.05 (s, 3H), 2.39 (s, 3H), 1.28 (s, 9H).

LCMS m/z 789 (M+H)⁺ (ES⁺)

### (ae) 4-((6-(5-((5-(tert-Butyl)-2-methoxy-3-(methylsulfonamido)phenyl)carbamoyl)-2-methyl-phenyl)quinazolin-2-yl)amino)-2-methoxy-N-(2-(2-methyl-1H-imidazol-1-yl)ethyl)benzamide

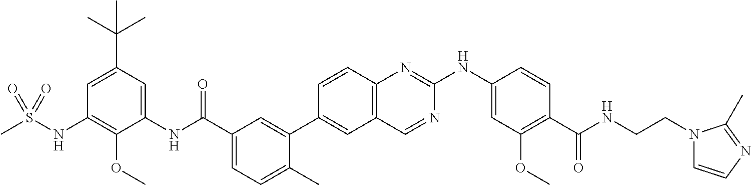 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCn1ccnc1C]

¹H NMR (400 MHz, DMSO-d6) δ 10.29 (s, 1H), 9.90 (s, 1H), 9.44 (s, 1H), 9.16 (s, 1H), 8.23 (d, 1H), 8.14 (t, 1H), 8.07-7.90 (m, 4H), 7.85 (dd, 2H), 7.59-7.50 (m, 2H), 7.47 (d, 1H), 7.25 (d, 1H), 7.06 (d, 1H), 6.76 (d, 1H), 4.08 (t, 2H), 3.96 (s, 3H), 3.72 (s, 3H), 3.60 (q, 2H), 3.06 (s, 3H), 2.39 (s, 3H), 2.30 (s, 3H), 1.28 (s, 9H).

LCMS m/z 791 (M+H)⁺ (ES⁺)

### (af) N-(2-(1H-Imidazol-4-yl)ethyl)-4-((6-(5-((5-(tert-butyl)-2-methoxy-3-(methylsulfonamido)-phenyl)carbamoyl)-2-methylphenyl)quinazolin-2-yl)amino)-2-methoxybenzamide

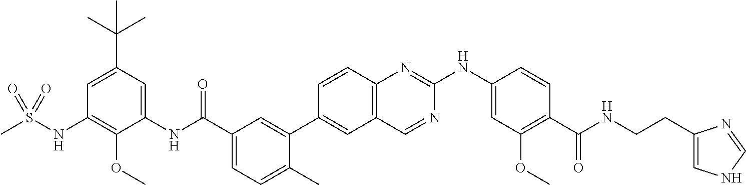 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCc1c[nH]cn1]

¹H NMR (400 MHz, DMSO-d6) δ 11.87 (s, 1H), 10.27 (s, 1H), 9.90 (s, 1H), 9.44 (s, 1H), 9.20 (s, 1H), 8.35 (s, 1H), 8.22 (d, 1H), 8.07-7.92 (m, 4H), 7.87 (dd, 2H), 7.67-7.58 (m, 1H), 7.59-7.48 (m, 2H), 7.48 (d, 1H), 7.26 (d, 1H), 6.90 (s, 1H), 3.97 (s, 3H), 3.72 (s, 3H), 3.56 (q, 2H), 3.06 (s, 3H), 2.77 (t, 2H), 2.39 (s, 3H), 1.28 (s, 9H).

LCMS m/z 777 (M+H)⁺ (ES⁺)

### (ag) 4-((6-(5-((5-(tert-Butyl)-2-methoxy-3-(methylsulfonamido)phenyl)carbamoyl)-2-methylphenyl)quinazolin-2-yl)amino)-2-methoxy-N-(2-(pyridin-3-yl)ethyl)benzamide

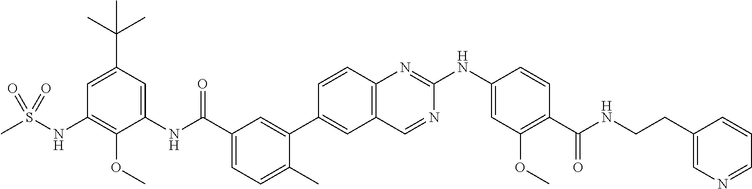 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCc1cccnc1]

¹H NMR (400 MHz, DMSO-d6) δ 10.27 (s, 1H), 9.90 (s, 1H), 9.44 (s, 1H), 9.16 (s, 1H), 8.54-8.48 (m, 1H), 8.46 (dd, 1H), 8.21 (d, 1H), 8.10 (t, 1H), 8.07-7.92 (m, 4H), 7.84 (dd, 2H), 7.71 (dt, 1H), 7.58-7.50 (m, 2H), 7.47 (d, 1H), 7.36 (ddd, 1H), 7.25 (d, J=2.4 Hz, 1H), 3.94 (s, 3H), 3.72 (s, 3H), 3.58 (q, 2H), 3.06 (s, 3H), 2.89 (t, 2H), 2.39 (s, 3H), 1.28 (s, 9H).

LCMS m/z 788 (M+H)⁺ (ES⁺)

### (ah) 4-((6-(5-((5-(tert-Butyl)-2-methoxy-3-(methylsulfonamido)phenyl)carbamoyl)-2-methylphenyl)quinazolin-2-yl)amino)-N-(2-(4-hydroxypiperidin-1-yl)ethyl)-2-methoxybenzamide

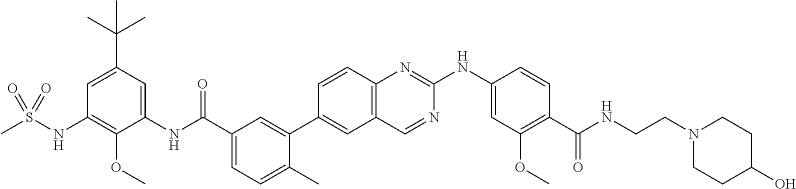 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCN1CCC(O)CC1]

¹H NMR (400 MHz, DMSO-d6) δ 10.28 (s, 1H), 9.89 (s, 1H), 9.44 (d, 1H), 9.14 (s, 1H), 8.32 (t, 1H), 8.25 (d, 1H), 8.04 (d, 1H), 8.01-7.82 (m, 5H), 7.58-7.49 (m, 2H), 7.47 (d, 1H), 7.25 (d, 1H), 4.59 (d, 1H), 4.04 (s, 3H), 3.71 (s, 3H), 3.49 (d, 1H), 3.40 (q, 2H), 3.05 (s, 3H), 2.77 (d, 2H), 2.47 (t, 2H), 2.40 (s, 3H), 2.09 (t, 2H), 1.77 (d, 2H), 1.44 (d, 2H), 1.28 (s, 9H).

LCMS m/z 810 (M+H)⁺ (ES⁺); 808 (M−H)⁻ (ES⁻)

### (ai) 4-((6-(5-((5-(tert-Butyl)-2-methoxy-3-(methylsulfonamido)phenyl)carbamoyl)-2-methylphenyl)quinazolin-2-yl)amino)-2-methoxy-N-((1-methylpiperidin-4-yl)methyl)benzamide

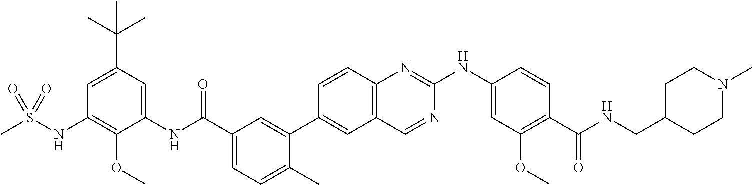

¹H NMR (400 MHz, DMSO-d6) δ 10.26 (s, 1H), 9.89 (s, 1H), 9.44 (s, 1H), 9.14 (s, 1H), 8.23 (d, 1H), 8.09-7.91 (m, 5H), 7.88-7.79 (m, 2H), 7.53 (d, 2H), 7.47 (d, 1H), 7.25 (d, 1H), 4.00 (s, 3H), 3.71 (s, 3H), 3.21 (t, 2H), 3.05 (s, 3H), 2.77 (s, 2H), 2.39 (s, 3H), 2.16 (s, 3H), 1.84 (s, 2H), 1.65 (d, 2H), 1.51 (s, 1H), 1.28 (s, 11H).

LCMS m/z 794 (M+H)⁺ (ES⁺); 792 (M−H)⁻ (ES⁻)

### (aj) 4-((6-(5-((5-(tert-Butyl)-2-methoxy-3-(methylsulfonamido)phenyl)carbamoyl)-2-methylphenyl)quinazolin-2-yl)amino)-N-(2-(4-hydroxy-1-methylpiperidin-4-yl)ethyl)-2-methoxybenzamide

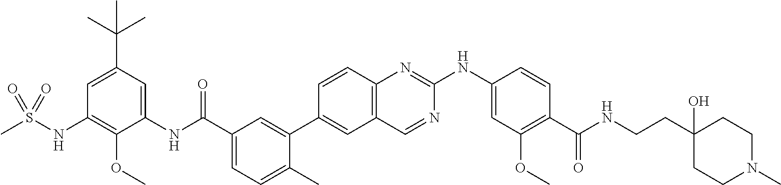

¹H NMR (400 MHz, DMSO-d6) δ 10.26 (s, 1H), 9.88 (s, 1H), 9.44 (d, 1H), 9.15 (s, 1H), 8.32 (t, 1H), 8.22 (d, 1H), 8.04 (d, 1H), 8.03-7.93 (m, 3H), 7.86 (dd, 2H), 7.56-7.50 (m, 2H), 7.46 (d, 1H), 7.25 (d, 1H), 4.26 (s, 1H), 3.99 (s, 3H), 3.71 (s, 3H), 3.42 (q, 2H), 3.04 (s, 3H), 2.43-2.25 (m, 7H), 2.16 (s, 3H), 1.65 (t, 2H), 1.52 (d, 4H), 1.28 (s, 9H).

LCMS m/z 824 (M+H)⁺ (ES⁺)

### (ak) 4-((6-(5-((5-(tert-Butyl)-2-methoxy-3-(methylsulfonamido)phenyl)carbamoyl)-2-methylphenyl)quinazolin-2-yl)amino)-2-methoxy-N-(2-(pyrrolidin-1-yl)ethyl)benzamide

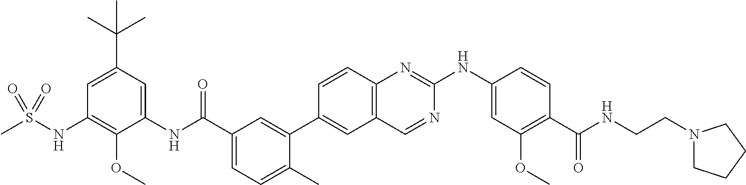 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCN1CCCC1]

¹H NMR (400 MHz, DMSO-d6) δ 10.28 (s, 1H), 9.88 (s, 1H), 9.44 (s, 1H), 9.15 (s, 1H), 8.31 (t, 1H), 8.23 (d, 1H), 8.04 (d, 1H), 8.02-7.94 (m, 3H), 7.87 (dd, 2H), 7.57-7.50 (m, 2H), 7.46 (d, 1H), 7.25 (d, 1H), 4.00 (s, 3H), 3.71 (s, 3H), 3.41 (q, 2H), 3.05 (s, 3H), 2.61 (t, 2H), 2.52 (s, 4H), 2.39 (s, 3H), 1.74 (p, 4H), 1.28 (s, 9H). 4 aliphatic signals obscured under DMSO signal.

LCMS m/z 780 (M+H)⁺ (ES⁺); 778 (M−H)⁻ (ES⁻)

### (al) 4-((6-(5-((5-(tert-Butyl)-2-methoxy-3-(methylsulfonamido)phenyl)carbamoyl)-2-methyl-phenyl)quinazolin-2-yl)amino)-2-methoxy-N-(2-(3,3,4-trimethylpiperazin-1-yl)ethyl)benzamide

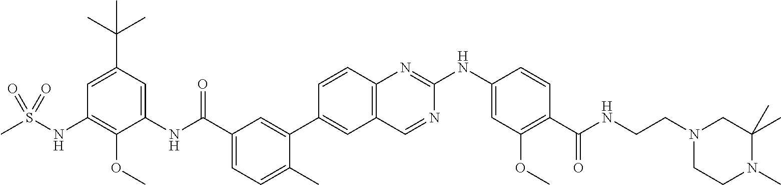

¹H NMR (400 MHz, DMSO-d6) δ 10.28 (s, 1H), 9.88 (s, 1H), 9.44 (d, 1H), 9.15 (s, 1H), 8.26 (d, 1H), 8.19 (t, 1H), 8.04 (d, 1H), 8.01-7.84 (m, 5H), 7.57-7.51 (m, 2H), 7.46 (d, 1H), 7.25 (d, 1H), 4.02 (s, 3H), 3.71 (s, 3H), 3.41 (q, 2H), 3.30 (s, 2H), 3.04 (s, 3H), 2.49-2.36 (m, 9H), 2.12 (s, 3H), 1.28 (s, 9H), 1.00 (s, 6H).

LCMS m/z 837 (M+H)⁺ (ES⁺); 835 (M−H)⁻ (ES⁻)

### (am) 4-((6-(5-((5-(tert-Butyl)-2-methoxy-3-(methylsulfonamido)phenyl)carbamoyl)-2-methylphenyl)quinazolin-2-yl)amino)-2-methoxy-N-(2-morpholinoethyl)benzamide

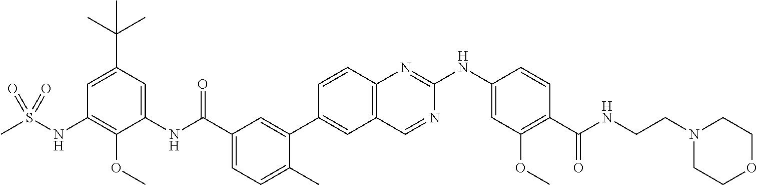 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NCCN1CCOCC1]

¹H NMR (400 MHz, DMSO-d6) δ 10.28 (s, 1H), 9.88 (s, 1H), 9.44 (d, 1H), 9.15 (s, 1H), 8.31 (s, 1H), 8.24 (d, 1H), 8.04 (d, 1H), 8.03-7.93 (m, 3H), 7.88 (dd, 2H), 7.59-7.50 (m, 2H), 7.46 (d, 1H), 7.25 (d, 1H), 4.04 (s, 3H), 3.71 (s, 3H), 3.64 (t, 4H), 3.43 (d, 2H), 3.05 (s, 3H), 2.51-2.42 (m, 6H), 2.40 (s, 3H), 1.28 (s, 9H).

LCMS m/z 796 (M+H)⁺ (ES⁺); 794 (M−H)⁻ (ES⁻)

### (an) 4-((6-(5-((5-(tert-Butyl)-2-methoxy-3-(methylsulfonamido)phenyl)carbamoyl)-2-methylphenyl)quinazolin-2-yl)amino)-N-(1,1-dioxidotetrahydro-2H-thiopyran-4-yl)-2-methoxybenzamide

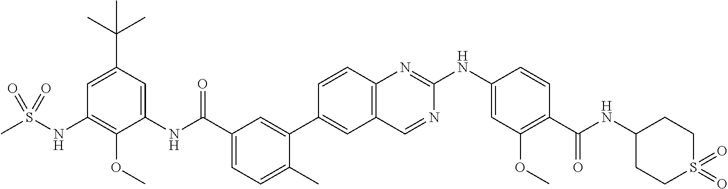 [SMILES: COc1cc(Nc2ncc3cc(-c4cc(C(=O)Nc5cc(C(C)(C)C)cc(NS(C)(=O)=O)c5OC)ccc4C)ccc3n2)ccc1C(=O)NC1CCS(=O)(=O)CC1]

¹H NMR (400 MHz, DMSO-d6) δ 10.27 (d, 1H), 9.89 (s, 1H), 9.44 (s, 1H), 9.14 (s, 1H), 8.22 (d, 1H), 8.05 (d, 2H), 8.02-7.92 (m, 3H), 7.84 (d, 1H), 7.75 (d, 1H), 7.53 (d, 2H), 7.47 (d, 1H), 7.25 (d, 1H), 4.19 (d, 1H), 4.00 (s, 3H), 3.71 (s, 3H), 3.11 (d, 4H), 3.05 (s, 3H), 2.39 (s, 3H), 2.22-2.06 (m, 4H), 1.28 (s, 9H).

LCMS m/z 815 (M+H)⁺ (ES⁺)

### Example 15

The following compounds are prepared by methods analogous to those described above.

### (a) [4-[[6-[5-[[5-tert-Butyl-3-(methanesulfonamido)-2-methoxy-phenyl]carbamoyl]-2-methyl-phenyl]quinazolin-2-yl]amino]-2-methoxy-phenyl]-methyl-phosphinic acid

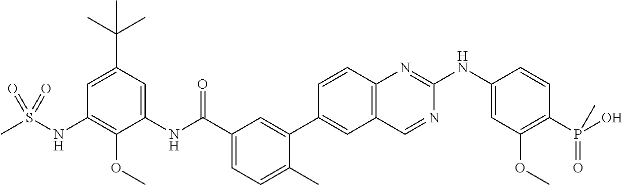 [SMILES: COc1cc(Nc2ncc3cc(-c4cc(C(=O)Nc5cc(C(C)(C)C)cc(NS(C)(=O)=O)c5OC)ccc4C)ccc3n2)ccc1P(C)(=O)O]

### (b) 4-[[6-[5-[[5-tert-Butyl-3-(methanesulfonamido)-2-methoxy-phenyl]carbamoyl]-2-methyl-phenyl]quinazolin-2-yl]amino]-2,6-dimethoxy-N-[2-(4-methylpiperazin-1-yl)ethyl]benzamide

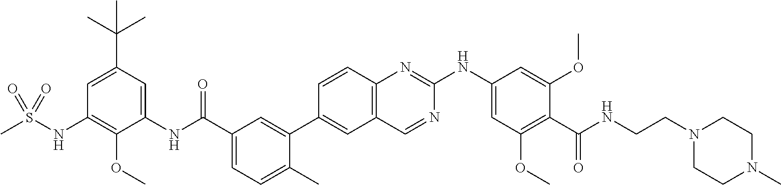

### (c) 4-[[6-[5-[[5-tert-Butyl-3-(methanesulfonamido)-2-methoxy-phenyl]carbamoyl]-2-methyl-phenyl]quinazolin-2-yl]amino]-N-(2-dimethylaminoethyl)-2,6-dimethoxy-benzamide

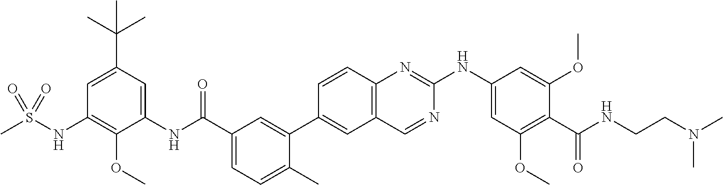

### (d) 2-[5-tert-Butyl-2-methoxy-3-[[3-[2-[3-methoxy-4-[2-(1-oxo-1,4-thiazinan-4-yl)ethylcarbamoyl]anilino]quinazolin-6-yl]-4-methyl-benzoyl]amino]-N-methylsulfonyl-anilino]ethyl dihydrogen phosphate

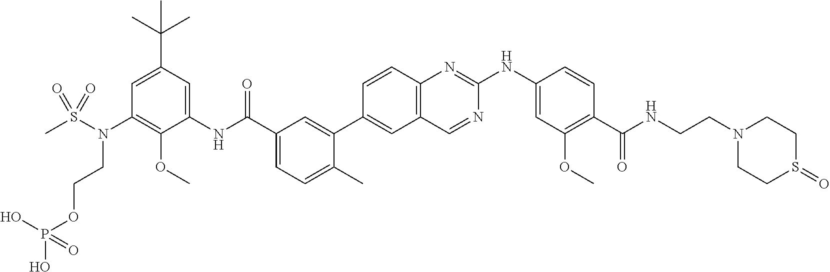 [SMILES: COc1cc(Nc2ncc3cc(-c4cc(C(=O)Nc5cc(C(C)(C)C)cc(N(CCOP(=O)(O)O)S(C)(=O)=O)c5OC)ccc4C)ccc3n2)ccc1C(=O)NCCN1CCS(=O)CC1]

### (e) 2-[[4-[[6-[5-[[5-tert-Butyl-3-(methanesulfonamido)-2-methoxy-phenyl]carbamoyl]-2-methyl-phenyl]quinazolin-2-yl]amino]-2,6-dimethoxy-benzoyl]amino]ethyl dihydrogen phosphate

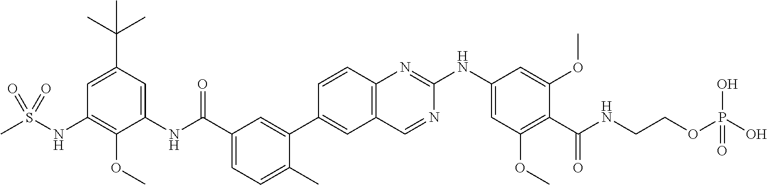 [SMILES: COc1cc(Nc2ncc3cc(-c4cc(C(=O)Nc5cc(C(C)(C)C)cc(NS(C)(=O)=O)c5OC)ccc4C)ccc3n2)cc(OC)c1C(=O)NCCOP(=O)(O)O]

### (f) 2-[2-[2-[[4-[[6-[5-[[5-tert-Butyl-3-(methanesulfonamido)-2-methoxy-phenyl]carbamoyl]-2-methyl-phenyl]quinazolin-2-yl]amino]-2,6-dimethoxy-benzoyl]amino]ethoxy]ethoxy]ethyl dihydrogen phosphate

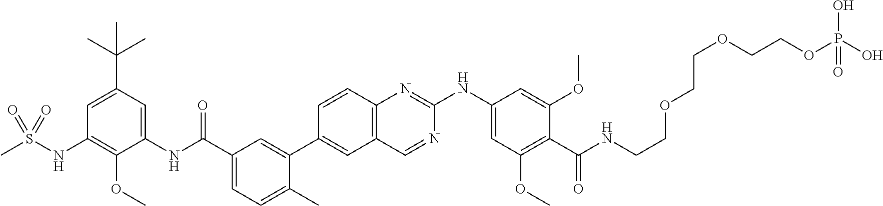 [SMILES: COc1cc(Nc2ncc3cc(-c4cc(C(=O)Nc5cc(C(C)(C)C)cc(NS(C)(=O)=O)c5OC)ccc4C)ccc3n2)cc(OC)c1C(=O)NCCOCCOCCOP(=O)(O)O]

### (g) [2-[5-tert-Butyl-2-methoxy-3-[[3-[2-[3-methoxy-4-[2-(1-oxo-1,4-thiazinan-4-yl)ethylcarbamoyl]anilino]quinazolin-6-yl]-4-methyl-benzoyl]amino]anilino]-2-oxo-ethyl]dihydrogen phosphate

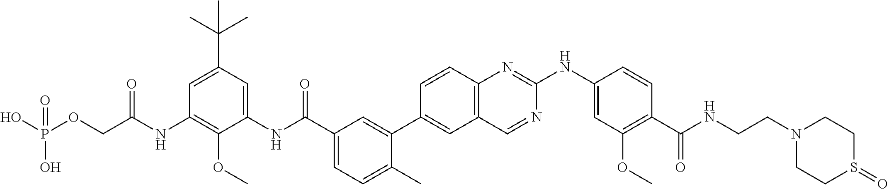 [SMILES: COc1cc(Nc2ncc3cc(-c4cc(C(=O)Nc5cc(C(C)(C)C)cc(NC(=O)COP(=O)(O)O)c5OC)ccc4C)ccc3n2)ccc1C(=O)NCCN1CCS(=O)CC1]

### Biological Testing: Experimental Methods

### Enzyme Binding Assays (Kinomescan)

Kinase enzyme binding activities of compounds disclosed herein may be determined using a proprietary assay which measures active site-directed competition binding to an immobilized ligand (Fabian, M. A. et al., Nature Biotechnol., 2005, 23:329-336). These assays may be conducted by DiscoverX (formerly Ambit; San Diego, Calif.). The percentage inhibition produced by incubation with a test compound may be calculated relative to the non-inhibited control.

### Enzyme Inhibition Assays

The enzyme inhibitory activities of compounds disclosed herein are determined by FRET using synthetic peptides labelled with both donor and acceptor fluorophores (Z-LYTE, Invitrogen Ltd., Paisley, UK).

### p38 MAPKα Enzyme Inhibition

The following two assay variants can be used for determination of p38 MAPKα inhibition.

### Method 1

The inhibitory activities of test compounds against the p38 MAPKα isoform (MAPK14: Invitrogen), are evaluated indirectly by determining the level of activation/phosphorylation of the down-stream molecule, MAPKAP-K2. The p38 MAPKα protein (80 ng/mL, 2.5 μL) is mixed with the test compound (2.5 μL of either 4 μg/mL, 0.4 μg/mL, 0.04 μg/mL or 0.004 μg/mL) for 2 hr at RT. The mix solution (2.5 μL) of the p38α inactive target MAPKAP-K2 (Invitrogen, 600 ng/mL) and FRET peptide (8 μM; a phosphorylation target for MAPKAP-K2) is then added and the kinase reaction is initiated by adding ATP (40 μM, 2.5 μL). The mixture is incubated for 1 hr at RT. Development reagent (protease, 5 μL) is added for 1 hr prior to detection in a fluorescence microplate reader (Varioskan® Flash, ThermoFisher Scientific).

### Method 2

This method follows the same steps as Method 1 above, but utilises a higher concentration of the p38 MAPKα protein (2.5 μL of 200 ng/mL protein instead of 2.5 μL of 80 ng/mL protein) for mixing with the test compound.

### p38 MAPKγ Enzyme Inhibition

The inhibitory activities of compounds of the invention against p38MAPKγ (MAPK12: Invitrogen), are evaluated in a similar fashion to that described hereinabove. The enzyme (800 ng/mL, 2.5 μL) is incubated with the test compound (2.5 μL at either 4 μg/mL, 0.4 μg/mL, 0.04 μg/mL, or 0.004 μg/mL) for 2 hr at RT. The FRET peptides (8 μM, 2.5 μL), and appropriate ATP solution (2.5 μL, 400 μM) is then added to the enzymes/compound mixtures and incubated for 1 hr. Development reagent (protease, 5 μL) is added for 1 hr prior to detection in a fluorescence microplate reader (Varioskan® Flash, Thermo Scientific).

c-Src and Syk Enzyme Inhibition

The inhibitory activities of compounds of the invention against c-Src and Syk enzymes (Invitrogen), are evaluated in a similar fashion to that described hereinabove. The relevant enzyme (3000 ng/mL or 2000 ng/mL respectively, 2.5 μL) is incubated with the test compound (either 4 μg/mL, 0.4 μg/mL, 0.04 μg/mL, or 0.004 μg/mL, 2.5 μL each) for 2 hr at RT. The FRET peptides (8 μM, 2.5 μL), and appropriate ATP solutions (2.5 μL, 800 μM for c-Src, and 60 μM ATP for Syk) are then added to the enzymes/compound mixtures and incubated for 1 hr. Development reagent (protease, 5 μL) is added for 1 hr prior to detection in a fluorescence microplate reader (Varioskan® Flash, ThermoFisher Scientific).

### GSK 3α Enzyme Inhibition

The following two assay variants can be used for determination of GSK 3α inhibition.

### Method 1

The inhibitory activities of compounds of the invention against the GSK 3α enzyme isoform (Invitrogen), are evaluated by determining the level of activation/phosphorylation of the target peptide. The GSK3-α protein (500 ng/mL, 2.5 μL) is mixed with the test compound (2.5 μL at either 4 μg/mL, 0.4 μg/mL, 0.04 μg/mL, or 0.004 μg/mL) for 2 hr at RT. The FRET peptide (8 μM, 2.5 μL), which is a phosphorylation target for GSK3α, and ATP (40 μM, 2.5 μL) are then added to the enzyme/compound mixture and the resulting mixture incubated for 1 hr. Development reagent (protease, 5 μL) is added for 1 hr prior to detection in a fluorescence microplate reader (Varioskan® Flash, ThermoFisher Scientific).

In all cases, the site-specific protease cleaves non-phosphorylated peptide only and eliminates the FRET signal. Phosphorylation levels of each reaction are calculated using the ratio of coumarin emission (donor) over fluorescein emission (acceptor), for which high ratios indicate high phosphorylation and low ratios indicate low phosphorylation levels. The percentage inhibition of each reaction is calculated relative to non-inhibited control and the 50% inhibitory concentration (IC₅₀ value) is then calculated from the concentration-response curve.

### Method 2

This method follows the same steps as Method 1 above, but utilises a shorter period of mixing of the test compound (105 minutes instead of 2 hours) with the GSK3-α protein.

### Cellular Assays

The compounds of the invention were studied using one or more of the following assays.

(a) LPS-Induced TNFα/IL-8 Release in d-U937 Cells

U937 cells, a human monocytic cell line, are differentiated to macrophage-type cells by incubation with phorbol myristate acetate (PMA; 100 ng/mL) for 48 to 72 hr. Cells are pre-incubated with final concentrations of test compound for 2 hr and are then stimulated with 0.1 μg/mL of LPS (from E. Coli: O111:B4, Sigma) for 4 hr. The supernatant is collected for determination of TNFα and IL-8 concentrations by sandwich ELISA (Duo-set, R&D systems). The inhibition of TNFα production is calculated as a percentage of that achieved by 10 μg/mL of BIRB796 at each concentration of test compound by comparison against vehicle control. The relative 50% effective concentration (REC₅₀) is determined from the resultant concentration-response curve. The inhibition of IL-8 production is calculated at each concentration of test compound by comparison with vehicle control. The 50% inhibitory concentration (IC₅₀) is determined from the resultant concentration-response curve.

### (b) LPS-Induced TNFα/IL-8 Release in PBMC Cells

Peripheral blood mononuclear cells (PBMCs) from healthy subjects are separated from whole blood using a density gradient (Lymphoprep, Axis-Shield Healthcare). The PBMCs are seeded in 96 well plates and treated with compounds at the desired concentration for 2 hours before addition of 1 ng/mL LPS (Escherichia Coli 0111:B4 from Sigma Aldrich) for 24 hours under normal tissue culture conditions (37° C., 5% CO₂). The supernatant is harvested for determination of IL-8 and TNFα concentrations by sandwich ELISA (Duo-set, R&D systems) and read on the fluorescence microplate reader (Varioskan® Flash, ThermoFisher Scientific). The concentration at 50% inhibition (IC₅₀) of IL-8 and TNFα production is calculated from the dose response curve.

### (c) IL-2 and IFN Gamma Release in CD3/CD28 Stimulated PBMC Cells

PBMCs from healthy subjects are separated from whole blood using a density gradient (Lymphoprep, Axis-Shield Healthcare). Cells are added to a 96 well plate pre-coated with a mixture of CD3/CD28 monoclonal antibodies (0.3 μg/mL eBioscience and 3 μg/mL BD Pharmingen respectively). Compound at the desired concentration is then added to the wells and the plate left for 3 days under normal tissue culture conditions. Supernatants are harvested and IL-2 and IFN gamma release determined by Sandwich ELISA (Duo-set, R&D System). The IC₅₀ is determined from the dose response curve.

### (d) IL-1β-Induced IL-8 Release in HT29 Cells

HT29 cells, a human colon adenocarcinoma cell line, are plated in a 96 well plate (24 hrs) and pre-treated with compounds at the desired concentration for 2 hours before addition of 5 ng/mL of IL-1β (Abcam) for 24 hours. Supernatants are harvested for IL-8 quantification by Sandwich ELISA (Duo-set, R&D System). The IC₅₀ is determined from the dose response curve.

### (e) LPS-Induced IL-8 and TNFα Release in Primary Macrophages

PBMCs from healthy subjects are separated from whole blood using a density gradient (Lymphoprep, Axis-Shield Healthcare). Cells are incubated for 2 hrs and non-adherent cells removed by washing. To differentiate the cells to macrophages the cells are incubated with 5 ng/mL of GM-CSF (Peprotech) for 7 days under normal tissue culture conditions. Compounds are then added to the cells at the desired concentration for a 2 hour pre-treatment before stimulation with 10 ng/mL LPS for 24 hours. Supernatants are harvested and IL-8 and TNFα release determined by Sandwich ELISA (Duo-set, R&D System). The IC₅₀ is determined from the dose response curve.

### (f) Poly I:C-Induced ICAM-1 Expression in BEAS2B Cells

Poly I:C is used in these studies as a simple, RNA virus mimic. Poly I:C-Oligofectamine mixture (1 μg/mL Poly I:C, ±2% Oligofectamine, 25 μL; Invivogen Ltd., San Diego, Calif., and Invitrogen, Carlsbad, Calif., respectively) is transfected into BEAS2B cells (human bronchial epithelial cells, ATCC). Cells are pre-incubated with final concentrations of test compounds for 2 hr and the level of ICAM1 expression on the cell surface is determined by cell-based ELISA. At a time point 18 hr after poly I:C transfection, cells are fixed with 4% formaldehyde in PBS and then endogenous peroxidase is quenched by the addition of washing buffer (100 μL, 0.05% Tween in PBS: PBS-Tween) containing 0.1% sodium azide and 1% hydrogen peroxide. Cells are washed with wash-buffer (3×200 μL) and after blocking the wells with 5% milk in PBS-Tween (100 μL) for 1 hr, the cells are incubated with anti-human ICAM-1 antibody (50 μL; Cell Signalling Technology, Danvers, Mass.) in 1% BSA PBS overnight at 4° C.

The cells are washed with PBS-Tween (3×200 μL) and incubated with the secondary antibody (100 μL; HRP-conjugated anti-rabbit IgG, Dako Ltd., Glostrup, Denmark). The cells are then incubated with of substrate (50 μL) for 2-20 min, followed by the addition of stop solution (50 μL, 1N H₂SO₄). The ICAM-1 signal is detected by reading the absorbance at 450 nm against a reference wavelength of 655 nm using a spectrophotometer. The cells are then washed with PBS-Tween (3×200 μL) and total cell numbers in each well are determined by reading absorbance at 595 nm after Crystal Violet staining (50 μL of a 2% solution in PBS) and elution by 1% SDS solution (100 μL) in distilled water. The measured OD 450-655 readings are corrected for cell number by dividing with the OD595 reading in each well. The inhibition of ICAM-1 expression is calculated at each concentration of test compound by comparison with vehicle control. The 50% inhibitory concentration (IC₅₀) is determined from the resultant concentration-response curve.

### (g) Cell Mitosis Assay

Peripheral blood mononucleocytes (PBMCs) from healthy subjects are separated from whole blood (Quintiles, London, UK) using a density gradient (Histopaque®-1077, Sigma-Aldrich, Poole, UK). The PBMCs (3 million cells per sample) are subsequently treated with 2% PHA (phytohaemagglutinin, Sigma-Aldrich, Poole, UK) for 48 hr, followed by a 20 hr exposure to varying concentrations of test compounds. At 2 hr before collection, PBMCs are treated with demecolcine (0.1 μg/mL; Invitrogen, Paisley, UK) to arrest cells in metaphase. To observe mitotic cells, PBMCs are permeabilised and fixed by adding Intraprep (50 μL; Beckman Coulter, France), and stained with anti-phospho-histone 3 (0.26 ng/L; #9701; Cell Signalling, Danvers, Mass.) and propidium iodide (1 mg/mL; Sigma-Aldrich, Poole, UK) as previously described (Muehlbauer P. A. and Schuler M. J., Mutation Research, 2003, 537:117-130). Fluorescence is observed using an ATTUNE flow cytometer (Invitrogen, Paisley, UK), gating for lymphocytes. The percentage inhibition of mitosis is calculated for each treatment relative to vehicle (0.5% DMSO) treatment.

### (h) Rhinovirus-Induced IL-8 Release and ICAM-1 Expression

Human rhinovirus RV16 is obtained from the American Type Culture Collection (Manassas, Va.). Viral stocks are generated by infecting Hela cells with HRV until 80% of the cells are cytopathic.

BEAS2B cells are infected with HRV at an MOI of 5 and incubated for 2 hr at 33° C. with gentle shaking for to promote absorption. The cells are then washed with PBS, fresh media added and the cells are incubated for a further 72 hr. The supernatant is collected for assay of IL-8 concentrations using a Duoset ELISA development kit (R&D systems, Minneapolis, Minn.).

The level of ICAM1 expressing cell surface is determined by cell-based ELISA. At 72 hr after infection, cells are fixed with 4% formaldehyde in PBS. After quenching endogenous peroxidase by adding 0.1% sodium azide and 1% hydrogen peroxide, wells are washed with wash-buffer (0.05% Tween in PBS: PBS-Tween). After blocking well with 5% milk in PBS-Tween for 1 hr, the cells are incubated with anti-human ICAM-1 antibody in 5% BSA PBS-Tween (1:500) overnight. Wells are washed with PBS-Tween and incubated with the secondary antibody (HRP-conjugated anti-rabbit IgG, Dako Ltd.). The ICAM-1 signal is detected by adding substrate and reading at 450 nm with a reference wavelength of 655 nm using a spectrophotometer. The wells are then washed with PBS-Tween and total cell numbers in each well are determined by reading absorbance at 595 nm after Crystal Violet staining and elution by 1% SDS solution. The measured OD₄₅₀₋₆₅₅ readings are corrected for cell number by dividing with the OD₅₉₅ reading in each well. Compounds are added 2 hr before HRV infection and 2 hr after infection when non-infected HRV is washed out.

### (i) Assessment of HRV16 Induced CPE in MRC5

MRC-5 cells are infected with HRV16 at an MOI of 1 in DMEM containing 5% FCS and 1.5 mM MgCl₂, followed by incubation for 1 hr at 33° C. to promote adsorption. The supernatants are aspirated, and then fresh media added followed by incubation for 4 days. Where appropriate, cells are pre-incubated with compound or DMSO for 2 hr, and the compounds and DMSO added again after washout of the virus.

Supernatants are aspirated and incubated with methylene blue solution (100 μL, 2% formaldehyde, 10% methanol and 0.175% Methylene Blue) for 2 hr at RT. After washing, 1% SDS in distilled water (100 μL) is added to each well, and the plates are shaken lightly for 1-2 hr prior to reading the absorbance at 660 nm. The percentage inhibition for each well is calculated. The IC₅₀ value is calculated from the concentration-response curve generated by the serial dilutions of the test compounds.

### (j) In Vitro RSV Virus Load in Primary Bronchial Epithelial Cells

Normal human bronchial epithelial cells (NHBEC) grown in 96 well plates are infected with RSV A2 (Strain A2, HPA, Salisbury, UK) at an MOI of 0.001 in the LHC8 Media:RPMI-1640 (50:50) containing 15 mM magnesium chloride and incubated for 1 hr at 37° C. for adsorption. The cells are then washed with PBS (3×200 μL), fresh media (200 μL) is added and incubation continued for 4 days. Where appropriate, cells are pre-incubated with the compound or DMSO for 2 hr, and then added again after washout of the virus.

The cells are fixed with 4% formaldehyde in PBS solution (50 μL) for 20 min, washed with WB (3×200 μL), (washing buffer, PBS including 0.5% BSA and 0.05% Tween-20) and incubated with blocking solution (5% condensed milk in PBS) for 1 hr. Cells are then washed with WB (3×200 μL) and incubated for 1 hr at RT with anti-RSV (2F7) F-fusion protein antibody (40 μL; mouse monoclonal, lot 798760, Cat. No. ab43812, Abcam) in 5% BSA in PBS-tween. After washing, cells are incubated with an HRP-conjugated secondary antibody solution (50 μL) in 5% BSA in PBS-Tween (lot 00053170, Cat. No. P0447, Dako) and then TMB substrate added (50 μL; substrate reagent pack, lot 269472, Cat. No. DY999, R&D Systems, Inc.). This reaction is stopped by the addition of 2N H₂SO₄ (50 μL) and the resultant signal is determined colourimetrically (OD: 450 nm with a reference wavelength of 655 nm) in a microplate reader (Varioskan® Flash, ThermoFisher Scientific).

Cells are then washed and a 2.5% crystal violet solution (50 μL; lot 8656, Cat. No. PL7000, Pro-Lab Diagnostics) is applied for 30 min. After washing with WB, 1% SDS in distilled water (100 μL) is added to each well, and plates are shaken lightly on the shaker for 1 hr prior to reading the absorbance at 595 nm. The measured OD₄₅₀₋₆₅₅ readings are corrected to the cell number by dividing the OD₄₅₀₋₆₅₅ by the OD₅₉₅ readings. The percentage inhibition for each well is calculated and the IC₅₀ value is calculated from the concentration-response curve generated from the serial dilutions of compound.

### (k) Cell Viability Assay: MTT Assay

Differentiated U937 cells are pre-incubated with each test compound (final concentration 1 μg/mL or 10 μg/mL in 200 μL media indicated below) under two protocols: the first for 4 hr in 5% FCS RPMI1640 media and the second in 10% FCS RPMI1640 media for 24 h. The supernatant is replaced with new media (200 μL) and MTT stock solution (10 μL, 5 mg/mL) is added to each well. After incubation for 1 hr the media are removed, DMSO (200 μL) is added to each well and the plates are shaken lightly for 1 hr prior to reading the absorbance at 550 nm. The percentage loss of cell viability is calculated for each well relative to vehicle (0.5% DMSO) treatment. Consequently an apparent increase in cell viability for drug treatment relative to vehicle is tabulated as a negative percentage.

### (l) Human Biopsy Assay

Intestinal mucosa biopsies are obtained from the inflamed regions of the colon of IBD patients. The biopsy material is cut into small pieces (2-3 mm) and placed on steel grids in an organ culture chamber at 37° C. in a 5% CO₂/95% O₂ atmosphere in serum-free media. DMSO control or test compounds at the desired concentration are added to the tissue and incubated for 24 hr in the organ culture chamber. The supernatant is harvested for determination of IL-6, IL-8, IL-1β and TNFα levels by R&D ELISA. Percentage inhibition of cytokine release by the test compounds is calculated relative to the cytokine release determined for the DMSO control (100%).

### (m) Accumulation of β Catenin in d-U937 Cells

U937 cells, a human monocytic cell line, are differentiated into macrophage-type cells by incubation with PMA; (100 ng/mL) for between 48 to 72 hr. The cells are then incubated with either final concentrations of test compound or vehicle for 18 hr. The induction of β-catenin by the test compounds is stopped by replacing the media with 4% formaldehyde solution. Endogenous peroxide activity is neutralised by incubating with quenching buffer (100 μL, 0.1% sodium azide, 1% H₂O₂ in PBS with 0.05% Tween-20) for 20 min. The cells are washed with washing buffer (200 μL; PBS containing 0.05% Tween-20) and incubated with blocking solution (200 μL; 5% milk in PBS) for 1 hr, re-washed with washing buffer (200 μL) and then incubated overnight with anti-β-catenin antibody solution (50 μL) in 1% BSA/PBS (BD, Oxford, UK).

After washing with washing buffer (3×200 μL; PBS containing 0.05% Tween-20), cells are incubated with an HRP-conjugated secondary antibody solution (100 μL) in 1% BSA/PBS (Dako, Cambridge, UK) and the resultant signal is determined colourimetrically (OD: 450 nm with a reference wavelength of 655 nm) using TMB substrate (50 μL; R&D Systems, Abingdon, UK). This reaction is stopped by addition of 1N H₂SO₄ solution (50 μL). Cells are then washed with washing buffer and 2% crystal violet solution (50 μL) is applied for 30 min. After washing with washing buffer (3×200 μL), 1% SDS (100 μL) is added to each well and the plates are shaken lightly for 1 hr prior to measuring the absorbance at 595 nm (Varioskan® Flash, Thermo-Fisher Scientific).

The measured OD₄₅₀₋₆₅₅ readings are corrected for cell number by dividing the OD₄₅₀₋₆₅₅ by the OD₅₉₅ readings. The percentage induction for each well is calculated relative to vehicle, and the ratio of induction normalised in comparison with the induction produced by a standard control comprising of Reference Compound A (N-(4-(4-(3-(3-tert-butyl-1-p-tolyl-1H-pyrazol-5-yl)ureido)naphthalen-1-yloxy)pyridin-2-yl)-2-methoxyacetamide) (1 μg/mL) which is defined as 100%.

### (n) T Cell Proliferation

PBMCs from healthy subjects are separated from whole blood using a density gradient (Lymphoprep, Axis-Shield Healthcare). The lymphocyte fraction is first enriched for CD4+ T cells by negative magnetic cell sorting as per the manufacturer's instructions (Miltenyi Biotec 130-091-155). Naïve CD4+ T cells are then separated using positive magnetic selection of CD45RA+ cells using microbeads as per the manufacturer's instructions (130-045-901). Cells are plated at 2×10⁵ cells per well in 100 μL RPMI/10% FBS on 96 well flat bottomed plate (Corning Costar). 25 μL of test compound are diluted to the appropriate concentration (8× final conc.) in normal medium and added to duplicate wells on the plate to achieve a dose response range of 0.03 ng/mL-250 ng/mL. DMSO is added as a negative control. Plates are allowed to pre-incubate for 2 hours before stimulation with 1 μg/mL anti-CD3 (OKT3; eBioscience). After 72 h, the medium in each well is replaced with 150 μL of fresh medium containing 10 μM BrdU (Roche). After 16 h, the supernatant is removed, the plate is dried and the cells fixed by adding 100 μL of fix/denature solution to each well for 20 min as per the manufacturer's instructions (Roche). Plates are washed once with PBS before addition of the anti-BrdU detection antibody and incubated for 90 mins at room temperature. Plates are then washed gently 3× with the wash buffer supplied and developed by addition of 100 μL of substrate solution. The reaction is stopped by addition of 50 μL of 1 M H₂SO₄, and read for absorbance at 450 nm on a plate reader (Varioskan® Flash, ThermoFisher Scientific). The IC₅₀ is determined from the dose response curve.

### (o) IL-2 and IFNγ Release in CD3/CD28 Stimulated LPMC Cells from IBD Patients

Lamina propria mononuclear cells (LPMCs) are isolated and purified from inflamed IBD mucosa of surgical specimens or from normal mucosa of surgical specimens as follows:

The mucosa is removed from the deeper layers of the surgical specimens with a scalpel and cut in fragments 3-4 mm size. The epithelium is removed by washing the tissue fragments three times with 1 mM EDTA (Sigma-Aldrich, Poole, UK) in HBSS (Sigma-Aldrich) with agitation using a magnetic stirrer, discarding the supernatant after each wash. The sample is subsequently treated with type 1A collagenase (1 mg/mL; Sigma-Aldrich) for 1 h with stirring at 37° C. The resulting cell suspension is then filtered using a 100 μm cell strainer, washed twice, resuspended in RPMI-1640 medium (Sigma-Aldrich) containing 10% fetal calf serum, 100 U/mL penicillin and 100 μg/mL streptomycin, and used for cell culture.

Freshly isolated LPMCs (2×10⁵ cells/well) are stimulated with 1 μg/mL α-CD3/α-CD28 for 48 h in the presence of either DMSO control or appropriate concentrations of compound. After 48 h, the supernatant is removed and assayed for the presence of TNFα and IFNγ by R&D ELISA. Percentage inhibition of cytokine release by the test compounds is calculated relative to the cytokine release determined for the DMSO control (100%).

(p) Inhibition of Cytokine Release from Myofibroblasts Isolated from IBD Patients

Myofibroblasts from inflamed IBD mucosa are isolated as follows:

The mucosa is dissected and discarded and 1 mm-sized mucosal samples are cultured at 37° C. in a humidified CO₂ incubator in Dulbecco's modified Eagle's medium (DMEM, Sigma-Aldrich) supplemented with 20% FBS, 1% non-essential amino acids (Invitrogen, Paisley, UK), 100 U/mL penicillin, 100 μg/mL streptomycin, 50 μg/mL gentamycin, and 1 μg/mL amphotericin (Sigma-Aldrich). Established colonies of myofibroblasts are seeded into 25-cm² culture flasks and cultured in DMEM supplemented with 20% FBS and antibiotics to at least passage 4 to provide a sufficient quantity for use in stimulation experiments.

Subconfluent monolayers of myofibroblasts are then seeded in 12-well plates at 3×10⁵ cells per well are starved in serum-free medium for 24 h at 37° C., 5% CO₂ before being cultured for 24 h in the presence of either DMSO control or appropriate concentrations of compound. After 24 h the supernatant is removed and assayed for the presence of IL-8 and IL-6 by R&D ELISA. Percentage inhibition of cytokine release by the test compounds is calculated relative to the cytokine release determined for the DMSO control (100%).

### (q) Human Neutrophil Degranulation

Neutrophils are isolated from human peripheral blood as follows:

Blood is collected by venepuncture and anti-coagulated by addition of 1:1 EDTA:sterile phosphate buffered saline (PBS, no Ca+/Mg+). Dextran (3% w/v) is added (1 part dextran solution to 4 parts blood) and the blood allowed to stand for approximately 20 minutes at rt. The supernatant is carefully layered on a density gradient (Lymphoprep, Axis-Shield Healthcare) and centrifuged (15 mins, 2000 rpm, no brake). The supernatant is aspirated off and the cell pellet is re-suspended in sterile saline (0.2%) for no longer than 60 seconds (to lyse contaminating red blood cells). 10 times volume of PBS is then added and the cells centrifuged (5 mins, 1200 rpm). Cells are re-suspended in HBSS+ (Hanks buffered salt solution (without phenol red) containing cytochalasin B (5 μg/mL) and 1 mM CaCl₂) to achieve 5×10⁶ cells/mL.

5×10⁴ cells are added to each well of a V-bottom 96 well plate and incubated (30 mins, 37° C.) with the appropriate concentration of test compound (0.3-1000 ng/mL) or vehicle (DMSO, 0.5% final conc). Degranulation is stimulated by addition of fMLP (final conc 1 μM) which after a further incubation (30 mins, 37° C.) the cells are removed by centrifugation (5 mins, 1500 rpm) and the supernatants transferred to a flat bottom 96 well plate. An equal volume of tetramethylbenzidine (TMB) is added and after 10 mins the reaction terminated by addition of an equal volume of sulphuric acid (0.5 M) and absorbance read at 450 nm (background at 655 nm subtracted). The 50% inhibitory concentration (IC₅₀) is determined from the resultant concentration-response curve.

### (r) Cell Cytotoxicity Assay

1×10⁵ Jurkat cells (immortalised human T lymphocytes) are added to the appropriate number of wells of a 96 well plate in 100 μL of media (RPMI supplemented with 10% foetal bovine serum). 1 μL of DMSO control (final concentration 1.0% v/v) or test compound (final concentration 20, 5 or 1 μg/mL) is added to the wells and incubated at 37° C., 5% CO₂. After 24 hours, the plate is centrifuged at 1200 rpm for 3 minutes and the supernatant discarded. Cells are then resuspended in 150 μL (final concentration 7.5 μg/mL) of propidium iodide (PI) in PBS and incubated at 37° C., 5% CO₂ for 15 minutes. After 15 minutes, cells are analysed by flow cytometry (BD accuri) using the FL3 window. The % viability is calculated as the % of cells that are PI negative in the test wells normalised to the DMSO control.

### In Vivo Screening: Pharmacodynamics and Anti-Inflammatory Activity

### (i) LPS-Induced Neutrophil Accumulation in Mice

Non-fasted Balb/c mice are dosed by the intra tracheal route with either vehicle, or the test substance at the indicated times (within the range 2-8 hr) before stimulation of the inflammatory response by application of an LPS challenge. At T=0, mice are placed into an exposure chamber and exposed to LPS (7.0 mL, 0.5 mg/mL solution in PBS) for 30 min. After a further 8 hr the animals are anesthetized, their tracheas cannulated and BALF extracted by infusing and then withdrawing from their lungs 1.0 mL of PBS via the tracheal catheter. Total and differential white cell counts in the BALF samples are measured using a Neubauer haemocytometer. Cytospin smears of the BALF samples are prepared by centrifugation at 200 rpm for 5 min at RT and stained using a DiffQuik stain system (Dade Behring). Cells are counted using oil immersion microscopy. Data for neutrophil numbers in BAL are shown as mean±S.E.M. (standard error of the mean). The percentage inhibition of neutrophil accumulation is calculated for each treatment relative to vehicle treatment.

### (ii) Cigarette Smoke Model

A/J mice (males, 5 weeks old) are exposed to cigarette smoke (4% cigarette smoke, diluted with air) for 30 min/day for 11 days using a Tobacco Smoke Inhalation Experiment System for small animals (Model SIS-CS; Sibata Scientific Technology, Tokyo, Japan). Test substances are administered intra-nasally (35 μL of solution in 50% DMSO/PBS) once daily for 3 days after the final cigarette smoke exposure. At 12 hr after the last dosing, each of the animals is anesthetized, the trachea cannulated and bronchioalveolar lavage fluid (BALF) is collected. The numbers of alveolar macrophages and neutrophils are determined by FACS analysis (EPICS® ALTRA II, Beckman Coulter, Inc., Fullerton, Calif., USA) using anti-mouse MOMA2 antibody (macrophage) or anti-mouse 7/4 antibody (neutrophil).

(iii) DSS-Induced Colitis in Mice

Non-fasted, 10-12 week old, male BDF1 mice are dosed by oral gavage twice daily with either vehicle, reference item (5-ASA) or test compound one day before (Day −1) stimulation of the inflammatory response by treatment with dextran sodium sulphate (DSS). On Day 0 of the study DSS (5% w/v) is administered in the drinking water followed by BID dosing of the vehicle (5 mL/kg), reference (100 mg/kg) or test compound (5 mg/kg) for 7 days. The drinking water with DSS is replenished every 3 days. During the study animals are weighed every day and stool observations are made and recorded as a score, based on stool consistency. At the time of sacrifice on Day +6 the large intestine is removed and the length and weight are recorded. Sections of the colon are taken for either MPO analysis to determine neutrophil infiltration or for histopathology scoring to determine disease severity.

### (iv) TNBS-Induced Colitis in Mice

Non-fasted, 10-12 week old, male BDF1 mice are dosed by oral gavage twice daily with either vehicle (5 mL/kg), reference item (Budesonide 2.5 mg/kg) or test compound (1 or 5 mg/kg) one day before (Day −1) stimulation of the inflammatory response by treatment with 2,4,6-trinitrobenzenesulphonic acid (TNBS) (15 mg/mL in 50% ethanol/50% saline). On Day 0 of the study TNBS (200 μL) is administered intra-colonically via a plastic catheter with BID dosing of the vehicle, reference or test compound continuing for 2 or 4 days. During the study animals are weighed every day and stool observations are made and recorded as a score, based on stool consistency. At the time of sacrifice on Day 2 (or Day 4) the large intestine is removed and the length and weight recorded. Sections of the colon are taken for histopathology scoring to determine disease severity.

### (v) Adoptive Transfer in Mice

On Study day 0, female Balb/C mice are terminated and spleens obtained for CD45RB^{high} cell isolation (Using SCID IBD cell Separation protocol). Approximately 4×10⁵ cells/mL CD45RB^{high} cells are then injected IP (100 μL/mouse) into female SCID animals. On study day 14, mice are weighed and randomized into treatment groups based on body weight. On Day 14, compounds are administered BID, via oral gavage, in a dose volume of 5 mL/kg. Treatment continues until study day 42, at which point the animals are necropsied 4 hours after am administration. The colon length and weight is recorded and used as a secondary endpoint in the study as a measurement of colon oedema. The colon is then divided into six cross-sections, four of which are used for histopathology scoring (primary endpoint) and two are homogenised for cytokine analysis. Data shown is the % inhibition of the induction window between naïve animals and vehicle animals, where higher inhibition implies closer to the non-diseased, naïve, phenotype.

### (vi) Endotoxin-Induced Uveitis in Rats

Male, Lewis rats (6-8 weeks old, Charles River UK Limited) are housed in cages of 3 at 19-21° C. with a 12 h light/dark cycle (07:00/19:00) and fed a standard diet of rodent chow and water ad libitum. Non-fasted rats are weighed, individually identified on the tail with a permanent marker and receive a single intravitreal administration into the right vitreous humor (5 μL dose volume) of 100 ng/animal, i.v.t. of LPS (Escherichia coli 0111:B4 prepared in PBS, Sigma Aldrich, UK) using a 32-gauge needle. Untreated rats are injected with PBS. Test compound, dexamethasone (Dex) or vehicle (20% hydroxypropyl-β-cyclodextrin, 0.1% HPMC, 0.01% Benzalconium chloride, 0.05% EDTA, 0.7% NaCl in deionised water) are administered by the topical route onto the right eye (10 μL) of animals 30 minutes prior to LPS, at the time of LPS administration, and 1, 2 and 4 hours post LPS administration. Before administration, the solution or suspension to be administered is agitated for 5 minutes to ensure a uniform suspension. 6 hours after LPS dosing, animals are euthanized by overdose with pentobarbitone (i.v.). Following euthanasia, the right eye of each animal is enucleated and dissected into front (anterior) and back (posterior) sections around the lens. Each section is weighed and homogenised in 500 μL of sterile phosphate buffered saline followed by 20 minutes centrifugation at 12000 rpm at 4° C. The resulting supernatant is divided into 3 aliquots and stored at −80° C. until subsequent cytokine analysis by R&D DuoSet ELISA.

### Summary of In Vitro and In Vivo Screening Results

TABLE 1a
Dissociation constants for selected kinases determined by LeadHunter
Discover Services (DiscoveRx Corporation, Fremont, CA), using the
KINOMEscan ™ technology.

| Test Compound | Dissociation Constant (nM) | | |
|---|---|---|---|
| Example No. | Lck | p38 MAPKα | Syk |
| 2 | 13 | 15 | 68 |
| 3 | 6.7 | 3.9 | 50 |

TABLE 1b
Results from in vitro p38 MAPKα (Method 2), c-Src, Syk and
GSK3α (Method 2) inhibition assays

| Test Compound | IC50 Values for Enzyme Inhibition (nM) | | | |
|---|---|---|---|---|
| Example No. | p38 MAPKα | c-Src | Syk | GSK3α |
| 1 | 776 | 14 | >1448 | >14478 |
| 2 | 32 | 27 | >1247 | >12470 |
| 3 | 12 | 18 | 781 | >13066 |
| 4 | — | — | — | >12839 |
| 5 | — | — | — | >12564 |
| 10 | 24 | 20 | >1163 | — |
| 12 | 8 | 4 | 190 | — |

TABLE 2
Results from cellular assays in PBMCs and HT29 cells (the protocols
for which are described by assays (b) to (d) above).

IC₅₀ Values for Inhibition
of Cytokine Release (nM)

| Test Compound | PBMCs | | | HT29 cells |
|---|---|---|---|---|
| Example No. | IL-8 | IL-2 | IFNγ | IL-8 |
| 1 | 133.7 | — | — | — |
| 2 | 26.0 | 125.9 | 53.3 | 78.7 |
| 3 | 8.0 | 16.5 | 18.4 | — |
| 4 | 14.8 | — | — | — |
| 5 | 10.3 | 38.7 | 30.4 | — |
| 7 | 3.8 | — | — | — |
| 8 | 4.0 | — | — | — |
| 9 | 3.1 | — | — | — |
| 10 | 3.5 | — | 13.1 | — |
| 11 | 7.6 | — | — | — |
| 12 | 2.3 | — | 12.6 | — |
| 13 | 6.7 | — | — | — |
| 14(a) | 31.1 | — | — | — |
| 14(b) | 15.2 | — | — | — |
| 14(c) | 18.6 | — | — | — |
| 14(d) | 8.3 | — | — | — |
| 14(e) | 20.6 | — | — | — |
| 14(f) | 10.4 | — | — | — |
| 14(g) | 17.3 | — | — | — |
| 14(h) | 9.4 | — | — | — |
| 14(i) | 6.9 | — | — | — |
| 14(j) | 9.1 | — | — | — |
| 14(k) | 16.7 | — | — | — |
| 14(l) | 16.6 | — | — | — |
| 14(m) | 10.4 | — | — | — |
| 14(n) | 11.0 | — | — | — |
| 14(o) | 28.4 | — | — | — |
| 14(p) | 7.5 | — | — | — |
| 14(q) | 6.1 | — | — | — |
| 14(r) | 49.1 | — | — | — |
| 14(s) | 35.6 | — | — | — |
| 14(t) | 67.6 | — | — | — |
| 14(u) | 20.2 | — | — | — |
| 14(v) | 4.4 | — | — | — |
| 14(w) | 8.4 | — | — | — |
| 14(x) | 7.7 | — | — | — |
| 14(y) | 4.6 | — | — | — |
| 14(z) | 6.0 | — | — | — |
| 14(aa) | 9.5 | — | — | — |
| 14(ab) | 5.4 | — | — | — |
| 14(ac) | 5.1 | — | — | — |
| 14(ad) | 7.0 | — | — | — |
| 14(ae) | 6.5 | — | — | — |
| 14(af) | 13.2 | — | — | — |
| 14(ag) | 11.6 | — | — | — |
| 14(ah) | 8.0 | — | — | — |
| 14(ai) | 7.9 | — | — | — |
| 14(aj) | 9.3 | — | — | — |
| 14(ak) | 14.8 | — | — | — |
| 14(al) | 8.3 | — | — | — |
| 14(am) | 19.5 | — | — | — |
| 14(an) | 12.3 | — | — | — |

As illustrated in Table 3 below, the compound of Example 3 of the present invention is markedly less active than the Reference Compound A (N-(4-(4-(3-(3-tert-butyl-1-p-tolyl-1H-pyrazol-5-yl)ureido)naphthalen-1-yloxy)pyridin-2-yl)-2-methoxyacetamide; WO 2010/112936) in assay (g) above, which measures impact on cell division (mitosis) in PBMCs. Furthermore, compounds of the examples of the present invention are substantially less cytotoxic than Reference Compound A, displaying enhanced viabilities in cell cytotoxicity assay (r) above (Table 3).

TABLE 3
Effect of compounds of the examples on cell division in PBMCs
and on Jurkat cell viability (NT = not tested).

| | % Inhibition | | | |
|---|---|---|---|---|
| Test compound | of mitosis | % Viability | % Viability | % Viability |
| Example No. | at 5 μg/mL | at 1 μg/mL | at 5 μg/mL | at 20 μg/mL |
| Reference | 87.8^{a} | 23.5 | 18.9 | 17.3 |
| Compound A | | | | |
| 3 | 66.3 | 100.4 | 82.9 | 77.5 |
| 5 | NT | 116.5 | 48.8 | 44.6 |
| 7 | NT | 79.0 | 54.7 | 49.7 |
| 8 | NT | 91.1 | 65.8 | 30.6 |
| 9 | NT | 63.0 | 38.4 | 28.7 |
| 10 | NT | 91.5 | 82.5 | 77.8 |
| 12 | NT | 86.4 | 79.3 | 81.0 |
| 14(g) | NT | 110.1 | 75.5 | 65.4 |
| 14(i) | NT | 69.8 | 8.4 | 2.1 |

^{a}See, for example, the value reported in WO 2013/050757.

### Summary of Additional Studies

### Hydrolytic Stability Study

Chemical stability of compounds of the invention was assessed in a mixture of DMSO and water (3:1) at a test compound concentration of 1 mg/mL

General HPLC Procedure
Agilent, Waters X-Select C18, 2.5 μm, 4.6×30 mm column, 4 min method, 5-95%
MeCN/water (0.1% formic acid).
Flow rate 2.5 ml/min.
Column Oven Temperature 40° C.
Detection 254 nm.

Sample Preparation
A 1.0 mg sample of test compound was dissolved in 1 mL of DMSO/water (3:1) and the resulting mixture was stirred at rt overnight.

Recording Stability
A 50 μL aliquot of the test solution was removed and analysed in duplicate by 5 μL HPLC injections. The peak area for the test compound (taken to be 100% product, 0% degradation) was recorded following manual integration of the corresponding UV trace. The area retention time was also recorded.
The test solution was heated to 60° C. internal temperature (65° C. block temperature), with stirring, and timing commenced once the target temperature had been reached. After 5 and 24 h, samples were removed and analysed by HPLC (5 μL injections). In all cases, 5 μL injections were used and the samples analysed in duplicate.
The peak areas for the test compounds were recorded at both subsequent timepoints and the % decomposition calculated from the % change in peak area over time. The remaining product was calculated as a percentage of the original value.
Reference Compound B (3-ethynyl-5-((4-((4-(3-(3-isopropyl-1-(p-tolyl)-1H-pyrazol-5-yl)ureido)naphthalen-1-yl)oxy)pyrimidin-2-yl)amino)-N-(2-morpholinoethyl)benzamide; Cariou, C. A. M., et al, WO 2014/027209) was included in each stability study as a control to validate the study.

The results of the study are reported in Table 4 below. In contrast to the compound of Example 2, the Reference Compound underwent substantial decomposition under the conditions of the experiment.

TABLE 4
Results from hydrolytic stability study.

| Test Compound | Time (min) | % Parent Remaining |
|---|---|---|
| Reference Compound B | 0 | 100^{a} (100^{b}) |
| | 300 | 93.3^{a} (71.0^{b}) |
| | 1440 | 52.3^{a} (26.0^{b}) |
| Example 2 | 0 | 100 |
| | 300 | 104.2 |
| | 1440 | 102.9 |

Key
^{a}Results for Reference Compound B from the experiment in which the compound of Example 2 was tested.
^{b}Cumulative average of results for Reference Compound B from multiple experiments.

### Abbreviations

AcOH glacial acetic acid
aq aqueous
5-ASA 5-aminosalicylic acid
ATP adenosine-5′-triphosphate
BALF bronchioalveolar lavage fluid
BID bis in die (twice-daily)
BINAP 2,2′-bis(diphenylphosphino)-1,1′-binaphthyl
BOP (benzotriazol-1-yloxy)tris(dimethylamino)phosphonium hexafluorophosphate
br broad
BrdU 5-bromo-2′-deoxyuridine
BSA bovine serum albumin
CatCart® catalytic cartridge
CDI 1,1-carbonyl-diimidazole
COPD chronic obstructive pulmonary disease
d doublet
dba dibenzylideneacetone
DBU 1,8-diazabicyclo[5.4.0]undec-7-ene
DCC dicyclohexylcarbodiimide
DCM dichloromethane
DIAD diisopropyl azodicarboxylate
DIPEA diisopropylethylamine
DMAP 4-dimethylaminopyridine
DMEM Dulbecco's modified eagle medium
DMF N,N-dimethylformamide
DMSO dimethyl sulfoxide
DPPA diphenylphosphoryl azide
d-U937 cells PMA differentiated U-937 cells
EDTA ethylenediaminetetraacetic acid
ELISA enzyme-linked immunosorbent assay
(ES⁻) electrospray ionization, negative mode
(ES⁺) electrospray ionization, positive mode
Et ethyl
Et₃N triethylamine
EtOAc ethyl acetate
EtOH ethanol
FACS fluorescence-activated cell sorting
FBS foetal bovine serum
FCS foetal calf serum
fMLP formyl-methionyl-leucyl-phenylalanine
FRET fluorescence resonance energy transfer
GSK3α glycogen synthase kinase 3α
HBEC primary human bronchial epithelial cells
HBSS Hank's balanced salt solution
HPLC high performance liquid chromatography
HPMC hydroxypropylmethylcellulose
h or hr hour(s)
HATU 2-(1H-7-azabenzotriazol-1-yl)-1,1,3,3-tetramethyl uronium hexafluorophosphate
HOAt 1-hydroxy-7-azabenzotriazole
HOBt hydroxybenzotriazole
HRP horseradish peroxidase
HRV human rhinovirus
ICAM-1 inter-cellular adhesion molecule 1
IFNγ interferon-γ
IL interleukin
iPrOAc isopropyl acetate
JNK c-Jun N-terminal kinase
LC liquid chromatography
Lck lymphocyte-specific protein tyrosine kinase
LPS lipopolysaccharide
m multiplet
(M+H)⁺ protonated molecular ion
MAPK mitogen-activated protein kinase
MAPKAP-K2 mitogen-activated protein kinase-activated protein kinase-2
mCPBA meta-chloroperbenzoic acid
Me methyl
MeCN acetonitrile
MeOH methanol
MHz megahertz
min or mins minute(s)
MMAD mass median aerodynamic diameter
MOI multiplicity of infection
MPO myeloperoxidase
MTT 3-(4,5-dimethylthiazol-2-yl)-2,5-diphenyltetrazolium bromide
MS mass spectrometry
m/z mass-to-charge ratio
NMP N-methyl pyrrolidinone
NMR nuclear magnetic resonance (spectroscopy)
OD optical density
PBMC peripheral blood mononuclear cell
PBS phosphate buffered saline
Ph phenyl
PHA phytohaemagglutinin
PMA phorbol myristate acetate
pTSA 4-methylbenzenesulfonic acid (para-toluenesulfonic acid)
PyBOP (benzotriazol-1-yloxy)tripyrrolidinophosphonium hexafluorophosphate
q quartet
rt or RT room temperature
RP HPLC reverse phase high performance liquid chromatography
rpm revolutions per minute
RPMI Roswell Park Memorial Institute
RSV respiratory syncytial virus
s singlet
sat or satd saturated
SCID severe combined immunodeficiency
SCX solid supported cation exchange (resin)
SDS sodium dodecyl sulfate
S_{N}Ar nucleophilic aromatic substitution
Syk Spleen tyrosine kinase
t triplet
T3P 1-propanephosphonic acid cyclic anhydride
TBAF tetrabutylammonium fluoride
TBDMS tert-butyldimethylsilyl
TCID₅₀ 50% tissue culture infectious dose
TEA triethylamine
THF tetrahydrofuran
TFA trifluoroacetic acid
TGFβ transforming growth factor beta
TIPS triisopropylsilyl
TMB 3,3′,5,5′-tetramethylbenzidine
TMS-Cl trimethylsilyl chloride
TNFα tumor necrosis factor alpha

Prefixes n-, s-, i-, t- and tert- have their usual meanings: normal, secondary, iso, and tertiary.

1. A compound of formula I, 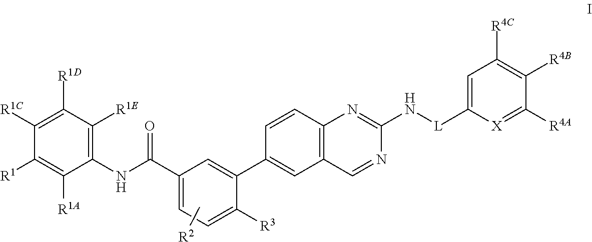
wherein
R¹ represents
-L¹-C(O)NR^{X}R^{Y},
-L²-S(O)₂R^{Y1},
-L³-P(O)R^{Y1}R^{Y2},
-L⁴-S(O)₂NR^{X}R^{Y},
-L⁵-C(O)R^{Y},
-L⁶-S(O)₀₋₁—R^{Y1},
-L⁷-C(O)OR^{Y},
—N═S(O)(CH₃)₂ or
—S(═O)(═NR^{X})CH₃
—NR^{X}R^{X1},
Het¹ optionally substituted with one or more substituents selected from the group consisting of halo, hydroxy, C₁₋₄ alkyl and C₁₋₄ alkoxy,
—[C₁₋₄ alkylene]₀₋₁-Het², which Het² group is optionally substituted with one or more substituents selected from the group consisting of halo, hydroxy, oxo, C₁₋₄ alkyl and C₁₋₄ alkoxy,
—[C₁₋₄ alkylene]₀₋₁—CN,
—C₁₋₄ alkylene-OH
—C₁₋₄ alkylene-OP(O)(OH)₂ or
halo
or, when either one or none of R^{4A} to R^{4C} represents H, then R¹ may alternatively represent H;
L¹, L², L³, L⁴, L⁵, L⁶ and L⁷ independently represent a bond, —[C(R^{a})(R^{b})]₁₋₂—, —C(R^{a})(R^{b})—N(R^{X2})—, where the C-atom is connected to the phenyl ring,
or L¹, L², L³, L⁴, L⁵ or L⁷ represents —N(R^{X2})—,
or L¹, L² or L⁴ represents —O—,
or L³ or L⁷ represents —OC(R^{a})(R^{b})—, wherein the O-atom of the latter substituent is attached to the phenyl ring;
R^{X} and R^{X1} independently represent H or C₁₋₆ alkyl optionally substituted by hydroxy or —OP(O)(OH)₂,
or R^{X1} represents Het¹ optionally substituted with one or more substituents selected from the group consisting of halo, hydroxy, C₁₋₄ alkyl and C₁₋₄ alkoxy;
R^{Y}, R^{Y1} and R^{Y2} independently represent C₁₋₆ alkyl, C₃₋₇ cycloalkyl, phenyl, benzyl, Het¹ or Het², which latter six groups are optionally substituted by one or more substituents selected from the group consisting of halo, hydroxy, —OP(O)(OH)₂, C₁₋₄ alkyl, C₁₋₄ alkoxy, C(O)OH, —N(R^{c})(R^{d}) and Het³, which latter group is optionally substituted by C₁₋₄ alkyl,
or R^{Y} represents H,
each R^{X2} independently represents H or C₁₋₄ alkyl optionally substituted by hydroxy or —OP(O)(OH)₂;
R^{c} and R^{d} independently represent H, methyl or —C(R^{e})(R^{f})—C₁₋₃ alkyl, the C₁₋₃ alkyl portion of which latter group is optionally substituted by one or more hydroxy substituents;
R^{a}, R^{b}, R^{e} and R^{f} represent, independently at each occurrence, H or methyl;
R^{1A} represents
C₁₋₆ alkoxy, C₁₋₆ alkyl, C₂₋₆ alkenyl, C₂₋₆ alkynyl, which latter four groups are optionally substituted by one or more substituents selected from the group consisting of C₁₋₂ alkyl, halo, hydroxy, —OP(O)(OH)₂, C₁₋₂ alkoxy and —N(R^{c})(R^{d}) or Het⁴,
H, halo, cyano,
phenyl or Het¹, which latter two groups are optionally substituted with one or more substituents selected from the group consisting of C₁₋₄ alkyl and C₁₋₄ alkoxy;
R^{1C} and R^{1E} independently represent H, halo, cyano or methyl;
R^{1D} represents C₂₋₇ alkyl, C₂₋₇ alkenyl, C₂₋₇ alkynyl, C₃₋₇ cycloalkyl, phenyl, Het¹ or Het², which latter seven groups are optionally substituted by one or more substituents selected from the group consisting of C₁₋₄ alkyl, halo, cyano, hydroxy, —OP(O)(OH)₂ and C₁₋₄ alkoxy, or R^{1D} represents trimethylsilyl or trifluoromethyl;
R² represents H, C₁₋₄ alkyl, C₃₋₄ cycloalkyl or halo;
R³ represents C₁₋₂ alkyl optionally substituted by one or more halo atoms, or R³ represents H or halo;
L represents a direct bond or C₁₋₂ alkylene;
X represents CH or N;
one of R^{4A}, R^{4B} and R^{4C} represents R^{5a}, and each of the other two of R^{4A}, R^{4B} and R^{4C} independently represents R^{5b};
R^{5a} represents
-Q¹-C(R^{6c})(R^{6d})—[C₁₋₅ alkylene]-R^{6a}, which C₁₋₅ alkylene group is optionally substituted by oxo and/or by one or more substituents selected from the group consisting of halo and hydroxy,
-Q²-[C(R^{6c})(R^{6d})—(CH₂)₀₋₁CH₂—O]₁₋₁₂—CH₂(CH₂)₀₋₁CH₂—R^{6a},
-Q³-J-Het^{x}, which Het^{x} group is optionally substituted by one or more substituents selected from the group consisting of halo, hydroxyl, oxo, CO₂H, C₁₋₃ alkyl, C₁₋₃ alkoxy and C₁₋₃ hydroxyalkyl,
-J-S(O)ₙR^{6b},
-J-P(O)R^{6e}R^{6f},
-J-CO₂H,
—OS(O)₂R^{6e},
—OS(O)₂NH₂,
—N═S(O)R^{6e}R^{6f},
-J-COR^{6b},
—S(═O)(═NR^{6c})CH₃,
—OCONH₂,
—CH₂OH or
—CH₂OP(O)(OH)₂;
R^{5b} represents C₁₋₃ alkoxy or C₁₋₃ alkyl, which latter two groups are optionally substituted by hydroxy, —OP(O)(OH)₂ or one or more halo atoms, or R^{5b} represents —S(O)₁₋₂R^{6e}, —OS(O)₂R^{6e}, —N(R⁹)(R¹⁰), C₂₋₃ alkynyl, H, cyano, —C(O)N(R⁹)(R¹⁰), hydroxy or halo;
R^{6a} represents OR^{7a}, —S(O)₀₋₂R^{7aa}, —N(R^{7b})(R^{7c}) or CO₂H;
R^{6b} represents C₁₋₈ alkyl, C₃₋₈ cycloalkyl, phenyl, Het¹ or Het², which latter five groups are optionally substituted by one or more substituents selected from the group consisting of halo, hydroxyl, —OP(O)(OH)₂, C₁₋₄ alkyl and C₁₋₄ alkoxy and which Het² group is optionally substituted or further substituted by one or more oxo groups,
or, when n is 2 or R^{6b} is attached to -J-CO—, in which J is C₁₋₄ alkylene, R^{6b} may alternatively represent —N(R^{7b})(R^{7c});
R^{7a} to R^{7c} independently represent H, —C(R^{7d})(R^{7e})—C₁₋₃ alkylene-OH, —C(R^{7d})(R^{7e})—C₁₋₃ alkylene-OP(O)(OH)₂ or C₁₋₄ alkyl optionally substituted by one or more halo atoms, or R^{7b} and R^{7c}, together with the N-atom to which they are attached, form a 4- to 7-membered heterocyclic group that is fully saturated, partially unsaturated or fully aromatic and which heterocyclic group contains one N atom (the atom to which R^{7b} and R^{7c} are attached) and, optionally, one or more further heteroatoms selected from the group consisting of O, S and N, and which heterocyclic group is optionally substituted by one or more substituents selected from halo, hydroxy, oxo, C₁₋₄ alkyl, C₁₋₄ alkoxy and C₁₋₄ hydroxyalkyl,
or R^{7a} represents —P(O)(OH)₂;
R^{7aa} represents —C(R^{7d})(R^{7e})—C₁₋₃ alkylene-OH or C₁₋₄ alkyl optionally substituted by one or more halo atoms;
R^{6c}, R^{6d}, R^{7d} and R^{7e} independently represent H or methyl;
R^{6e} and R^{6f} represent, independently at each occurrence, C₁₋₄ alkyl optionally substituted with one or more halo atoms,
or, when R^{5a} represents -J-P(O)R^{6e}R^{6f}, R^{6e} and R^{6f} may additionally represent hydroxy or C₁₋₄ alkoxy;
Q¹, Q², and Q³ independently represent —C(O)N(R⁸)—, —O—, —S(O)₂N(R⁸)— or —S(O)ₚ—;
J represents a direct bond or C₁₋₄ alkylene;
n and p independently represent 0, 1 or 2;
R⁸, R⁹ and R¹⁰ independently represent H or methyl;
Het^{x} represents Het¹ or Het³;
Het¹ represents, independently upon each occurrence, a 5- or 6-membered heterocyclic group that is fully aromatic, which group contains one or more heteroatoms selected from the group consisting of N, O and S; and
Het² to Het⁴ represent, independently upon each occurrence, a 4- to 7-membered heterocyclic group that is fully saturated or partially unsaturated, which group contains one or more heteroatoms selected from the group consisting of N, O and S;
or a pharmaceutically acceptable salt, solvate or isotopic derivative thereof.

2. A compound according to claim 1 that is a compound of formula Ia, Ib, Ic, Id or Ie, 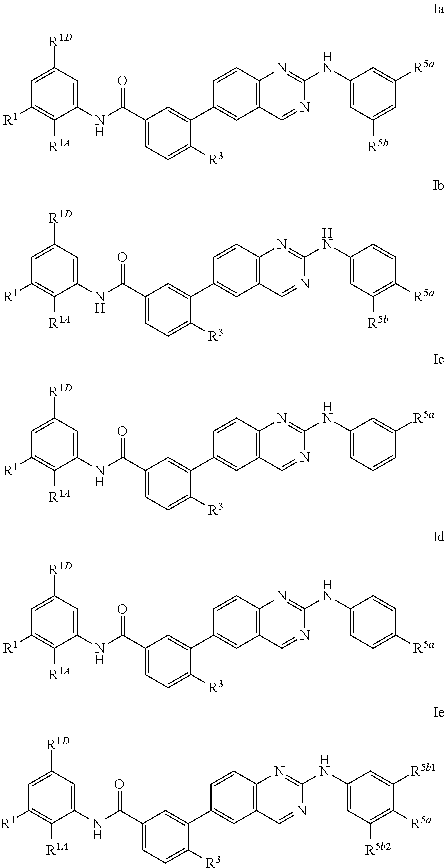
wherein R^{5b1} and R^{5b2} independently represent R^{5b} and R¹, R^{1A}, R^{1c}, R³, R^{5a} and R^{5b} are as defined in claim 1.

3. A compound according to claim 1, wherein R¹ represents —C(O)N(H)R^{Y}, —NHS(O)₂CH₃ or —P(O)(CH₃)₂.

4. A compound according to claim 1, wherein R^{Y} represents H.

5. A compound according to claim 1, wherein R^{1A} represents methoxy or ethoxy.

6. A compound according to claim 1, wherein R^{1D} represents tert-butyl.

7. A compound according to claim 1, wherein R³ represents methyl.

8. A compound according to claim 1, wherein R^{5a} represents
—C(O)NH—C(H)(R^{6c})—[C₁₋₄ alkylene]-R^{6a}, which C₁₋₃ alkylene group is optionally substituted by oxo or by one or two hydroxy,
-Q²-[C(H)(R^{6c})CH₂—O]₁₋₆—CH₂CH₂—R^{6a},
-Q³-J-Het^{x},
-J-P(O)(C₁₋₂ alkyl)₂,
—CO₂H,
—OS(O)₂CH₃,
—N═S(O)(CH₃)₂,
—CO-Het², which Het² group is optionally substituted by one or two oxo groups, or
—S(═O)(═NH)CH₃.

9. A compound according to claim 1, wherein R^{5b} represents halo, cyano, —C≡CH, —OS(O)₂CH₃, methyl or methoxy, which latter two groups are optionally substituted by one or more fluoro atoms.

10. A compound according to claim 1, wherein:
Q² represents —O— or —C(O)NH—;
Q³ represents —O—, —C(O)N(H)—, —C(O)N(CH₃)— or —S(O)₂N(CH₃)—; and/or
J represents a direct bond or C₁₋₃ alkylene.

11. A compound according to claim 1, wherein R^{6c} represents H or methyl.

12. A compound according to claim 1, wherein R^{6a} represents —OH, —OCH₃—S(O)₂CH₃, —CO₂H or —N(R^{7b})(R^{7c}).

13. A compound according to claim 1, wherein:
R^{7b} and R^{7c} both represent methyl, or R^{7b} and R^{7c}, together with the N-atom to which they are attached, form a morpholinyl, thiomorpholinyl, homomorpholinyl or pyrrolidinyl group, a piperazinyl group optionally substituted by one or more substituents selected from the group consisting of methyl and 2-hydroxyethyl, a piperidinyl group optionally substituted by hydroxy or a thiomorpholinyl group optionally substituted by one or more oxo groups; and/or
Het² represents a 5- or 6-membered heterocyclic group that is fully saturated or partially unsaturated, which group contains one or two heteroatoms selected from the group consisting of N, O and S.

14. A compound as claimed in claim 1, which is a compound selected from the group consisting of:
3-(2-((3-methoxy-5-(2-(2-(2-methoxyethoxy)ethoxy)ethoxy)phenyl)amino)quinazolin-6-yl)-4-methyl-N-(3-(trifluoromethyl)phenyl)benzamide;
N-(5-(tert-butyl)-2-methoxy-3-(methylsulfonamido)phenyl)-3-(2-((3-methoxy-5-(2-(2-(2-methoxyethoxy)ethoxy)ethoxy)phenyl)amino)quinazolin-6-yl)-4-methylbenzamide;
N-(5-(tert-butyl)-3-carbamoyl-2-methoxyphenyl)-3-(2-((3-methoxy-5-((2-morpholinoethyl)carbamoyl)phenyl)amino)quinazolin-6-yl)-4-methylbenzamide;
N-(5-(tert-butyl)-3-(dimethylphosphoryl)-2-methoxyphenyl)-3-(2-((3-methoxy-5-((2-morpholinoethyl)carbamoyl)phenyl)amino)quinazolin-6-yl)-4-methylbenzamide;
N-(5-(tert-butyl)-2-methoxy-3-(methylsulfonamido)phenyl)-3-(2-((3-methoxy-5-((2-morpholinoethyl)carbamoyl)phenyl)amino)quinazolin-6-yl)-4-methylbenzamide;
N-(5-tert-butyl-2-methoxy-3-methylsulfonyl-phenyl)-3-[2-[3-methoxy-5-(2-morpholinoethyl-carbamoyl)anilino]quinazolin-6-yl]-4-methyl-benzamide;
N-(5-tert-butyl-2-methoxy-3-methylsulfinyl-phenyl)-3-[2-[3-methoxy-5-(2-morpholinoethyl-carbamoyl)anilino]quinazolin-6-yl]-4-methyl-benzamide;
N-(5-(tert-butyl)-2-methoxy-3-(methylsulfonamido)phenyl)-3-(2-((4-((dimethyl(oxo)-I6-sulfanylidene)amino)phenyl)amino)quinazolin-6-yl)-4-methylbenzamide;
4-((6-(5-((5-(tert-butyl)-2-methoxy-3-(methylsulfonamido)phenyl)carbamoyl)-2-methylphenyl)quinazolin-2-yl)amino)-2-methoxybenzoic acid;
4-((6-(5-((5-(tert-butyl)-2-methoxy-3-(methylsulfonamido)phenyl)carbamoyl)-2-methylphenyl)quinazolin-2-yl)amino)-2-methoxy-N-methyl-N-(2-morpholinoethyl)benzamide;
3-((6-(5-((5-(tert-butyl)-2-methoxy-3-(methylsulfonamido)phenyl)carbamoyl)-2-methylphenyl)-quinazolin-2-yl)amino)-5-((2-morpholinoethyl)carbamoyl)phenyl methanesulfonate;
4-((6-(5-((5-(tert-butyl)-2-methoxy-3-(methylsulfonamido)phenyl)carbamoyl)-2-methylphenyl)-quinazolin-2-yl)amino)-2-methoxy-N-(3-(1-oxidothiomorpholino)propyl)benzamide;
N-(5-(tert-butyl)-2-methoxy-3-(methylsulfonamido)phenyl)-3-(2-((4-(1,1-dioxidothiomorpholine-4-carbonyl)-3-methoxyphenyl)amino)quinazolin-6-yl)-4-methylbenzamide;
4-((6-(5-((5-(tert-butyl)-2-methoxy-3-(methylsulfonamido)phenyl)carbamoyl)-2-methylphenyl)-quinazolin-2-yl)amino)-N-(2-(4-(2-hydroxyethyl)piperazin-1-yl)ethyl)-2-methoxybenzamide;
3-((6-(5-((5-(tert-butyl)-2-methoxy-3-(methylsulfonamido)phenyl)carbamoyl)-2-methylphenyl)quinazolin-2-yl)amino)phenyl methanesulfonate;
N-(5-(tert-butyl)-2-methoxy-3-(methylsulfonamido)phenyl)-3-(2-((3-cyano-5-(2-morpholinoethoxy)phenyl)amino)quinazolin-6-yl)-4-methylbenzamide;
N-(5-(tert-butyl)-2-methoxy-3-(methylsulfonamido)phenyl)-3-(2-((3-methoxy-5-(N-methyl-N-(2-morpholinoethyl)sulfamoyl)phenyl)amino)quinazolin-6-yl)-4-methylbenzamide;
N-(5-(tert-butyl)-2-methoxy-3-(methylsulfonamido)phenyl)-4-methyl-3-(2-((3-((2-morpholinoethyl)carbamoyl)phenyl)amino)quinazolin-6-yl)benzamide;
N-(5-(tert-butyl)-2-methoxy-3-(methylsulfonamido)phenyl)-3-(2-((3-methoxy-5-(N-methyl-N-(3-morpholinopropyl)sulfamoyl)phenyl)amino)quinazolin-6-yl)-4-methylbenzamide;
N-(5-(tert-butyl)-2-methoxy-3-(methylsulfonamido)phenyl)-3-(2-((3-cyano-5-(2-(2-(2-methoxyethoxy)ethoxy)ethoxy)phenyl)amino)quinazolin-6-yl)-4-methylbenzamide;
N-(5-(tert-butyl)-2-methoxy-3-(methylsulfonamido)phenyl)-3-(2-((3-ethynyl-5-((2-(2-(2-methoxyethoxy)ethoxy)ethyl)carbamoyl)phenyl)amino)quinazolin-6-yl)-4-methylbenzamide;
N-(5-(tert-butyl)-2-methoxy-3-(methylsulfonamido)phenyl)-3-(2-((3-(dimethylphosphoryl)phenyl)amino)quinazolin-6-yl)-4-methyl benzamide;
N-(5-(tert-butyl)-2-methoxy-3-(methylsulfonamido)phenyl)-3-(2-((3-((dimethylphosphoryl)methyl)phenyl)amino)quinazolin-6-yl)-4-methylbenzamide;
4-((6-(5-((5-(tert-butyl)-2-methoxy-3-(methylsulfonamido)phenyl)carbamoyl)-2-methylphenyl)quinazolin-2-yl)amino)phenyl methanesulfonate;
N-(5-(tert-butyl)-2-methoxy-3-(methylsulfonamido)phenyl)-3-(2-((3-ethynyl-5-(2-(2-(2-methoxyethoxy)ethoxy)ethoxy)phenyl)amino)quinazolin-6-yl)-4-methylbenzamide;
N-(5-(tert-butyl)-2-methoxy-3-(methylsulfonamido)phenyl)-3-(2-((3-fluoro-5-(2-(2-(2-methoxyethoxy)ethoxy)ethoxy)phenyl)amino)quinazolin-6-yl)-4-methylbenzamide;
N-(5-(tert-butyl)-2-methoxy-3-(methylsulfonamido)phenyl)-3-(2-((3-ethynyl-5-((2-(2-methoxyethoxy)ethyl)carbamoyl)phenyl)amino)quinazolin-6-yl)-4-methylbenzamide;
N-(5-(tert-butyl)-2-methoxy-3-(methylsulfonamido)phenyl)-3-(2-((3-ethynyl-5-((2-methoxyethyl)carbamoyl)phenyl)amino)quinazolin-6-yl)-4-methylbenzamide;
3-((6-(5-((5-(tert-butyl)-2-methoxy-3-(methylsulfonamido)phenyl)carbamoyl)-2-methylphenyl)quinazolin-2-yl)amino)-5-methoxybenzoic acid;
4-((6-(5-((5-(tert-butyl)-2-methoxy-3-(methylsulfonamido)phenyl)carbamoyl)-2-methylphenyl)-quinazolin-2-yl)amino)-2-methoxy-N-(2-(2-(2-methoxyethoxy)ethoxy)ethyl)benzamide;
N-(5-(tert-butyl)-2-methoxy-3-(methylsulfonamido)phenyl)-3-(2-((3-((dimethyl(oxo)-I6-sulfanylidene)amino)phenyl)amino)quinazolin-6-yl)-4-methylbenzamide;
N-(5-(tert-butyl)-2-methoxy-3-(methylsulfonamido)phenyl)-3-(2-((3-(2-(2-(2-methoxyethoxy)-ethoxy)ethoxy)-5-(trifluoromethoxy)phenyl)amino)quinazolin-6-yl)-4-methylbenzamide;
N-(5-(tert-butyl)-2-methoxy-3-(methylsulfonamido)phenyl)-3-(2-((3-(2-(2-(2-methoxyethoxy)ethoxy)ethoxy)-5-(trifluoromethyl)phenyl)amino)quinazolin-6-yl)-4-methylbenzamide;
N-(5-(tert-butyl)-2-methoxy-3-(methylsulfonamido)phenyl)-4-methyl-3-(2-((4-(S-methylsulfonimidoyl)phenyl)amino)quinazolin-6-yl)benzamide;
N-(5-(tert-butyl)-2-methoxy-3-(methylsulfonamido)phenyl)-4-methyl-3-(2-((3-((2-morpholinoethyl)carbamoyl)-5-(trifluoromethyl)phenyl)amino)quinazolin-6-yl)benzamide;
4-((6-(5-((5-(tert-butyl)-2-methoxy-3-(methylsulfonamido)phenyl)carbamoyl)-2-methylphenyl)quinazolin-2-yl)amino)-N-(2-(2-(2-hydroxyethoxy)ethoxy)ethyl)-2-methoxybenzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(2-(2-methoxyethoxy)ethyl)benzamide;
4-((6-(5-((5-(tert-butyl)-2-methoxy-3-(methylsulfonamido)phenyl)carbamoyl)-2-methylphenyl)-quinazolin-2-yl)amino)-N-(3-hydroxy-2,2-bis(hydroxymethyl) propyl)-2-methoxybenzamide;
N-(2-(1,4-oxazepan-4-yl)ethyl)-4-((6-(5-((5-(tert-butyl)-2-methoxy-3-(methylsulfonamido)-phenyl)carbamoyl)-2-methylphenyl)quinazolin-2-yl)amino)-2-methoxybenzamide;
4-((6-(5-((5-(tert-butyl)-2-methoxy-3-(methylsulfonamido)phenyl)carbamoyl)-2-methyl-phenyl)quinazolin-2-yl)amino)-2-methoxy-N-(2-(4-methylpiperazin-1-yl)ethyl)benzamide;
4-((6-(5-((5-(tert-butyl)-2-methoxy-3-(methylsulfonamido)phenyl)carbamoyl)-2-methyl-phenyl)quinazolin-2-yl)amino)-2-methoxy-N-(2-(1-methylpiperidin-4-yl)ethyl)benzamide;
N-(3-(1,4-oxazepan-4-yl)propyl)-4-((6-(5-((5-(tert-butyl)-2-methoxy-3-(methylsulfonamido)-phenyl)carbamoyl)-2-methylphenyl)quinazolin-2-yl)amino)-2-methoxybenzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(1-methylpiperidin-4-yl)benzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(2-(methylsulfonyl)ethyl)benzamide;
4-((6-(5-((5-(tert-butyl)-2-methoxy-3-(methylsulfonamido)phenyl)carbamoyl)-2-methyl-phenyl)quinazolin-2-yl)amino)-2-methoxy-N-(2-(2-methyl-1H-imidazol-1-yl)ethyl)benzamide;
N-(2-(1H-imidazol-4-yl)ethyl)-4-((6-(5-((5-(tert-butyl)-2-methoxy-3-(methylsulfonamido)-phenyl)carbamoyl)-2-methylphenyl)quinazolin-2-yl)amino)-2-methoxybenzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(2-(pyridin-3-yl)ethyl)benzamide;
4-((6-(5-((5-(tert-butyl)-2-methoxy-3-(methylsulfonamido)phenyl)carbamoyl)-2-methylphenyl)-quinazolin-2-yl)amino)-N-(2-(4-hydroxypiperidin-1-yl)ethyl)-2-methoxybenzamide;
4-((6-(5-((5-(tert-butyl)-2-methoxy-3-(methylsulfonamido)phenyl)carbamoyl)-2-methylphenyl)-quinazolin-2-yl)amino)-2-methoxy-N-((1-methylpiperidin-4-yl)methyl)benzamide;
4-((6-(5-((5-(tert-butyl)-2-methoxy-3-(methylsulfonamido)phenyl)carbamoyl)-2-methylphenyl)-quinazolin-2-yl)amino)-N-(2-(4-hydroxy-1-methylpiperidin-4-yl)ethyl)-2-methoxybenzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(2-(pyrrolidin-1-yl)ethyl)benzamide;
4-((6-(5-((5-(tert-butyl)-2-methoxy-3-(methylsulfonamido)phenyl)carbamoyl)-2-methylphenyl)-quinazolin-2-yl)amino)-2-methoxy-N-(2-(3,3,4-trimethylpiperazin-1-yl)ethyl)benzamide;
4-((6-(5-((5-(tert-butyl)-2-methoxy-3-(methylsulfonamido)phenyl)carbamoyl)-2-methylphenyl)quinazolin-2-yl)amino)-2-methoxy-N-(2-morpholinoethyl)benzamide;
4-((6-(5-((5-(tert-butyl)-2-methoxy-3-(methylsulfonamido)phenyl)carbamoyl)-2-methylphenyl)quinazolin-2-yl)amino)-N-(1,1-dioxidotetrahydro-2H-thiopyran-4-yl)-2-methoxybenzamide;
[4-[[6-[5-[[5-tert-butyl-3-(methanesulfonamido)-2-methoxy-phenyl]carbamoyl]-2-methyl-phenyl]quinazolin-2-yl]amino]-2-methoxy-phenyl]-methyl-phosphinic acid;
4-[[6-[5-[[5-tert-butyl-3-(methanesulfonamido)-2-methoxy-phenyl]carbamoyl]-2-methyl-phenyl]quinazolin-2-yl]amino]-2,6-dimethoxy-N-[2-(4-methylpiperazin-1-yl)ethyl]benzamide;
4-[[6-[5-[[5-tert-butyl-3-(methanesulfonamido)-2-methoxy-phenyl]carbamoyl]-2-methyl-phenyl]quinazolin-2-yl]amino]-N-(2-dimethylaminoethyl)-2,6-dimethoxy-benzamide;
2-[5-tert-butyl-2-methoxy-3-[[3-[2-[3-methoxy-4-[2-(1-oxo-1,4-thiazinan-4-yl)ethylcarbamoyl]-anilino]quinazolin-6-yl]-4-methyl-benzoyl]amino]-N-methylsulfonyl-anilino]ethyl dihydrogen phosphate;
2-[[4-[[6-[5-[[5-tert-butyl-3-(methanesulfonamido)-2-methoxy-phenyl]carbamoyl]-2-methyl-phenyl]quinazolin-2-yl]amino]-2,6-dimethoxy-benzoyl]amino]ethyl dihydrogen phosphate;
2-[2-[2-[[4-[[6-[5-[[5-tert-butyl-3-(methanesulfonamido)-2-methoxy-phenyl]carbamoyl]-2-methyl-phenyl]quinazolin-2-yl]amino]-2,6-dimethoxy-benzoyl]amino]ethoxy]ethoxy]ethyl dihydrogen phosphate; and
[2-[5-tert-butyl-2-methoxy-3-[[3-[2-[3-methoxy-4-[2-(1-oxo-1,4-thiazinan-4-yl)ethylcarbamoyl]anilino]quinazolin-6-yl]-4-methyl-benzoyl]amino]anilino]-2-oxo-ethyl]dihydrogen phosphate,
or a pharmaceutically acceptable salt, solvate or isotopic derivative thereof.

15. A pharmaceutical formulation comprising a compound as defined in claim 1, or pharmaceutically acceptable salt, solvate or isotopic derivative thereof, in admixture with a pharmaceutically acceptable adjuvant, diluent or carrier.

16. A combination product comprising:
(A) a compound as defined in claim 1, or pharmaceutically acceptable salt, solvate or isotopic derivative thereof, and
(B) another therapeutic agent,
wherein each of components (A) and (B) is formulated in admixture with a pharmaceutically-acceptable adjuvant, diluent or carrier.

17. (canceled)

18. (canceled)

19. (canceled)

20. A method of treating or preventing an inflammatory disease, said method comprising administering to a subject an effective amount of:
a compound as defined in claim 1, or pharmaceutically acceptable salt, solvate or isotopic derivative thereof,
a pharmaceutical formulation comprising a compound as defined in claim 1, or pharmaceutically acceptable salt, solvate or isotopic derivative thereof, in admixture with a pharmaceutically acceptable adjuvant, diluent or carrier, or
a combination product comprising:
(A) a compound as defined in claim 1, or pharmaceutically acceptable salt, solvate or isotopic derivative thereof, and
(B) another therapeutic agent,
wherein each of components (A) and (B) is formulated in admixture with a pharmaceutically-acceptable adjuvant, diluent or carrier.

21. The method according to claim 20, wherein the inflammatory disease is selected from the group consisting of ulcerative colitis, Crohn's disease, gluten sensitive enteropathy (coeliac disease), eosinophilic eosophagitis, intestinal graft versus host disease, conjunctivitis, allergic conjunctivitis, keratoconjunctivitis sicca (dry eye), glaucoma, diabetic retinopathy, macular oedema, diabetic macular oedema, central retinal vein occlusion (CRVO), dry and/or wet age related macular degeneration (AMD), post-operative cataract inflammation, uveitis, posterior uveitis, anterior uveitis pan uveitis, corneal graft and limbal cell transplant rejection, cystic fibrosis, pulmonary hypertension, lung sarcoidosis, idiopathic pulmonary fibrosis, COPD, chronic bronchitis emphysema, asthma, paediatric asthma, atopic dermatitis, allergic dermatitis, contact dermatitis, psoriasis, allergic rhinitis, rhinitis and sinusitis.

22. The method according to claim 10, wherein the inflammatory disease is ulcerative colitis, Crohn's disease, keratoconjunctivitis sicca (dry eye) or uveitis.

23. The method according to claim 20, wherein the inflammatory disease is asthma or COPD.

24. A process for the preparation of a compound of formula I which process comprises:
(a) reaction of a compound of formula II, 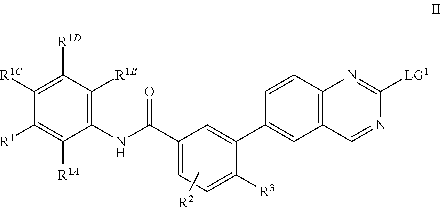
wherein LG¹ represents a leaving group and R¹, R^{1A}, R^{1C}, R^{1D}, R^{1E}, R² and R³ are as defined in claim 1 with a compound of formula III, 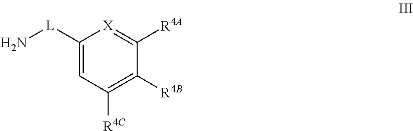
wherein L, X, R^{4A}, R^{4B} and R^{4C} are as defined in claim 1;
(b) reaction of a compound of formula IV, 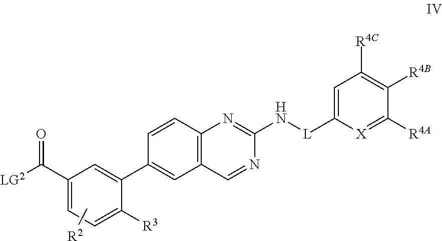
wherein LG² represents a leaving group and R², R³, L, X, R^{4A}, R^{4B} and R^{4C} are as defined in claim 1, with a compound of formula V, 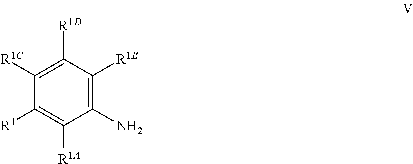
wherein R¹, R^{1A}, R^{1C}, R^{1D} and R^{1E} are as defined in claim 1;
(c) for compounds of formula I in which R¹ represents -L¹-C(O)NR^{X}R^{Y}, -L²-S(O)₂R^{Y1}, -L³-P(O)R^{Y1}R^{Y2}, -L⁴-S(O)₂NR^{X}R^{Y}, -L⁵-C(O)R^{Y} or -L⁷-C(O)OR^{Y}, in which L¹, L², L³, L⁴, L⁵ and L⁷ represent —N(R^{X2})— or —C(R^{a})(R^{b})—N(R^{X2})—, reaction of a compound of formula VI, 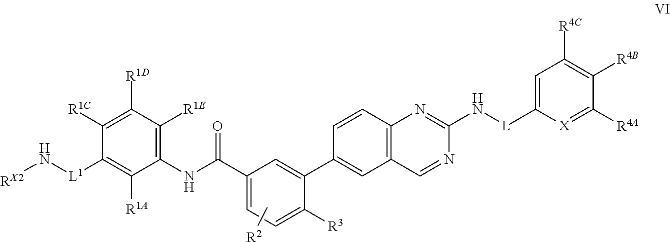
wherein L¹ represents a direct bond or —C(R^{a})(R^{b})— and R^{X2}, R^{1A}, R^{1C}, R^{1D}, R^{1E}, R², R³, L, X, R^{4A}, R^{4B} and R^{4C} are as defined in claim 1, with a compound of formula VIIa, VIIb, VIIc, VIId, VIIe or VIIf,
LG³-C(O)NR^{X}R^{Y} VIIa
LG³-S(O)₂R^{Y1} VIIb
LG³-P(O)R^{Y1}R^{Y2} VIIc
LG³-S(O)₂NR^{X}R^{Y} VIId
LG³-C(O)R^{Y} VIIe
LG³-C(O)OR^{Y} VIIf
wherein LG³ represents a leaving group and R^{X}, R^{Y}, R^{Y1} and R^{Y2} are as defined in claim 1;
(d) for compounds of formula I in which R¹ represents -L¹-C(O)NR^{X}R^{Y} in which L¹ represents a bond or —[C(R^{a})(R^{b})]₁₋₂—, reaction of a compound of formula VIII, 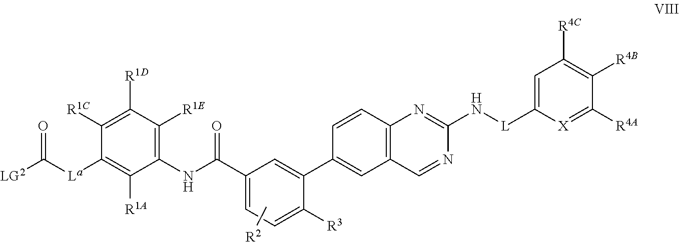
wherein LG² represents a leaving group, L^{a} represents a bond or —[C(R^{a})(R^{b})]₁₋₂- and R^{1A}, R^{1C}, R^{1D}, R^{1E}, R², R³, L, X, R^{4A}, R^{4B} and R^{4C} are as defined in claim 1, with a compound of formula IX,
HNR^{X}R^{Y} IX
wherein R^{X} and R^{Y} are as defined in claim;
(e) for compounds of formula I where
Q¹, Q² or Q³ represents —C(O)N(R⁸)— or —S(O)₂N(R⁸)— or
R^{5a} represents —C₁₋₄-alkylene-CO—N(R^{7b})R^{7c} or —S(O)₂—N(R^{7b})R^{7c}, condensation of a compound of formula VIIIa— 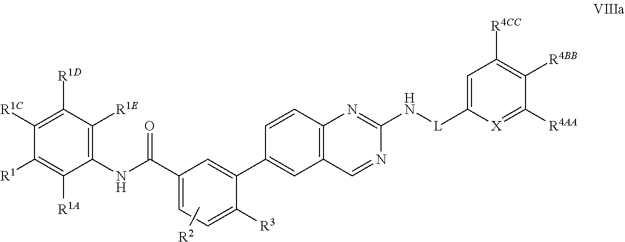
wherein one of R^{4AA}, R^{4BB} and R^{4CC} represents —[C₁₋₄-alkylene]₀₋₁—C(O)LG² or —S(O)₂LG² and each of the other two of R^{4AA}, R^{4BB} and R^{4CC} independently represents R^{5b} and R¹, R^{1A}, R^{1C}, R^{1D}, R^{1E}, R², R³, R^{5b}, L and X are as defined in claim 1 and LG² is as defined above, with an amine of formula IXa, IXb, IXc or IXd,
HNR⁸—C(R^{6c})(R^{6d})—[C₁₋₅ alkylene]-R^{6a1} IXa
HNR⁸—[C(R^{6c})(R^{6d})—(CH₂)₀₋₁CH₂—O]₁₋₁₂—CH₂(CH₂)₀₋₁CH₂—R^{6a1} IXb
HNR⁸-J-Het^{X} IXc
HN(R^{7b})R^{7c} IXd
which C₁₋₅ alkylene and Het^{X} groups are optionally substituted as described in claim 1, wherein R^{6c}, R^{6d}, R⁸, J and Het^{X} are as defined in claim 1, and R^{6a1} takes the same definition as R^{6a} in claim 1, except that CO₂H is only present in protected form;
(f) for compounds of formula I in which
R¹ represents —C₁₋₄ alkylene-OP(O)(OH)₂,
R^{X} and/or R^{X1} represents C₁₋₆ alkyl substituted by —OP(O)(OH)₂,
R^{Y}, R^{Y1} and/or R^{Y2} represents C₁₋₆ alkyl, C₃₋₇ cycloalkyl, phenyl, benzyl, Het¹ or Het², which latter six groups are substituted by —OP(O)(OH)₂ and are optionally further substituted by one or more substituents selected from the group consisting of halo, hydroxy, C₁₋₄ alkyl, C₁₋₄ alkoxy, C(O)OH, —N(R^{c})(R^{d}) and Het³, which latter group is optionally substituted by C₁₋₄ alkyl,
R^{X2} represents C₁₋₄ alkyl substituted by —OP(O)(OH)₂,
R^{1A} represents C₁₋₆ alkoxy, C₁₋₆ alkyl, C₂₋₆ alkenyl, C₂₋₆ alkynyl, which latter four groups are substituted by —OP(O)(OH)₂ and are optionally further substituted by one or more substituents selected from the group consisting of C₁₋₂ alkyl, halo, hydroxy, C₁₋₂ alkoxy and —N(R^{c})(R^{d}) or Het⁴,
R^{1D} represents C₂₋₇ alkyl, C₂₋₇ alkenyl, C₂₋₇ alkynyl, C₃₋₇ cycloalkyl, phenyl, Het¹ or Het², which latter seven groups are substituted by —OP(O)(OH)₂ and are optionally further substituted by one or more substituents selected from the group consisting of C₁₋₄ alkyl, halo, cyano, hydroxy and C₁₋₄ alkoxy, or R^{1D} represents trimethylsilyl or trifluoromethyl,
R^{5a} represents —CH₂OP(O)(OH)₂,
R^{5b} represents C₁₋₃ alkoxy or C₁₋₃ alkyl, which latter two groups are substituted by —OP(O)(OH)₂,
R^{6b} represents C₁₋₈ alkyl, C₃₋₈ cycloalkyl, phenyl, Het¹ or Het², which latter five groups are substituted by —OP(O)(OH)₂ and are optionally further substituted by one or more substituents selected from the group consisting of halo, hydroxyl, C₁₋₄ alkyl and C₁₋₄ alkoxy and which Het² group is optionally substituted or further substituted by one or more oxo groups,
R^{7a}, R^{7b} and/or R^{7c} represents —C(R^{7d})(R^{7e})—C₁₋₃ alkylene-OP(O)(OH)₂ or
R^{7a} represents —P(O)(OH)₂,
reaction of a hydroxy group on a corresponding compound of formula I in which, respectively
R¹ represents —C₁₋₄ alkylene-OH,
R^{X} and/or R^{X1} represents C₁₋₆ alkyl substituted by hydroxy,
R^{Y}, R^{Y1} and/or R^{Y2} represents C₁₋₆ alkyl, C₃₋₇ cycloalkyl, phenyl, benzyl, Het¹ or Het², which latter six groups are substituted by hydroxy and are optionally further substituted by one or more substituents selected from the group consisting of halo, hydroxy, C₁₋₄ alkyl, C₁₋₄ alkoxy, C(O)OH, —N(R^{c})(R^{d}) and Het³, which latter group is optionally substituted by C₁₋₄ alkyl,
R^{X2} represents C₁₋₄ alkyl substituted by hydroxy,
R^{1A} represents C₁₋₆ alkoxy, C₁₋₆ alkyl, C₂₋₆ alkenyl, C₂₋₆ alkynyl, which latter four groups are substituted by hydroxy and are optionally further substituted by one or more substituents selected from the group consisting of C₁₋₂ alkyl, halo, hydroxy, C₁₋₂ alkoxy and —N(R^{c})(R^{d}) or Het⁴,
R^{1D} represents C₂₋₇ alkyl, C₂₋₇ alkenyl, C₂₋₇ alkynyl, C₃₋₇ cycloalkyl, phenyl, Het¹ or Het², which latter seven groups are substituted by hydroxy and are optionally further substituted by one or more substituents selected from the group consisting of C₁₋₄ alkyl, halo, cyano, hydroxy and C₁₋₄ alkoxy, or R^{1D} represents trimethylsilyl or trifluoromethyl,
R^{5a} represents —CH₂OH,
R^{5b} represents C₁₋₃ alkoxy or C₁₋₃ alkyl, which latter two groups are substituted by hydroxy,
R^{6b} represents C₁₋₈ alkyl, C₃₋₈ cycloalkyl, phenyl, Het¹ or Het², which latter five groups are substituted by hydroxy and are optionally further substituted by one or more substituents selected from the group consisting of halo, hydroxyl, C₁₋₄ alkyl and C₁₋₄ alkoxy and which Het² group is optionally substituted or further substituted by one or more oxo groups,
R^{7a}, R^{7b} and/or R^{7c} represents —C(R^{7d})(R^{7e})—C₁₋₃ alkylene-OH or
R^{7a} represents H,
with di-tert-butyl diethylphosphoramidite or dibenzyl-N,N-diisopropylphosphoramidite, followed by reaction with an oxidant and then removal of the respective tert-butyl or benzyl protecting groups by hydrolysis using an acid or hydrogenolysis with a palladium catalyst;
(g) deprotection of a protected derivative of a compound of formula I.
